(19) United States
(12) Patent Application Publication
(10) Pub. No.: US 2010/0113613 A1
(43) Pub. Date: May 6, 2010

(54) CYCLOHEXANE POLYALCOHOL FORMULATION FOR TREATMENT OF DISORDERS OF PROTEIN AGGREGATION

(75) Inventors: JoAnne McLaurin, Toronto (CA); Antonio Cruz, Toronto (CA)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: WARATAH PHARMACEUTICALS, Toronto, ON (CA)

(21) Appl. No.: 12/282,030

(22) PCT Filed: Mar. 9, 2007
(86) PCT No.: PCT/CA2007/000395

§ 371 Date: Jan. 13, 2010

Related U.S. Application Data
(60) Provisional application No. 60/780,526, filed on Mar. 9, 2006.
(60) Provisional application No. 60/819,864, filed on Jul. 11, 2006.
(60) Provisional application No. 60/897,667, filed on Jan. 26, 2007.

(51) Int. Cl. A61K 31/047; C07C 35/14; A61P 25/28

(52) U.S. Cl. 514/738; 568/833

(57) ABSTRACT

The invention provides formulations, dosage forms, and treatments comprising cyclohexane polyalcohol compounds that provide beneficial pharmacokinetic profiles in the treatment of a disorder and/or disease including a disorder in protein folding and/or aggregation, and/or amyloid formation, deposition, accumulation, or persistence. In aspects of the invention, a dosage form is provided comprising an amount of a cyclohexane polyalcohol compound suitable for administration to a subject to provide a therapeutically effective concentration of the compound in plasma, brain and/or cerebral spinal fluid and a pharmaceutically acceptable carrier, diluent or excipient. The formulation can be administered in a dose of 500, 1000, 2000, 3500, 5000 or 7000 mg of cyclohexane polyalcohol compound to achieve a mean plasma concentration profile having a mean AUC_{0-INF} in μ·h/mL of, respectively, 43±20%, 130±20%, 215±20%, 467±20%, 507±20% or 885±20%, and having a mean Cₘₐₓ in μmL of, respectively, 5.8±20%, 17±20%, 33±20%, 75±20%, 110±20% or 155±20%.

### FIELD OF THE INVENTION

The invention relates generally to formulations, dosage forms, drug delivery systems or technologies and methods suitable to produce beneficial pharmacokinetic profiles of cyclohexane polyalcohol compounds for the treatment of disorders of protein aggregation.

### BACKGROUND OF THE INVENTION

Cyclohexane polyalcohol compounds hold potential as disease modifying treatments for Alzheimer's disease (AD). When given orally to a transgenic mouse model of Alzheimer's disease (AD), cyclohexanehexyl stereoisomers inhibit aggregation of amyloid β-peptide (Aβ) in the brain and ameliorate several AD-like phenotypes in the model, including impaired cognition, altered synaptic physiology, cerebral amyloid β and accelerated mortality. These effects occur regardless of whether the compounds are given prior to, or well after the onset of the AD-like phenotype. These compounds preferentially target the soluble oligomers of Aβ both in vitro and in vivo, and have no effects on amyloid precursor protein processing. (See US Published Application No. 20040204387.)

### SUMMARY OF THE INVENTION

The invention relates generally to dosage forms, formulations and methods that produce beneficial pharmacokinetic profiles of cyclohexane polyalcohol compounds, in particular scyllo-cyclohexanehexyl compounds and epi-cyclohexanehexyl compounds, for the treatment of a disorder and/or disease described herein, in particular a disorder in protein folding and/or aggregation, and/or amyloid formation, deposition, accumulation, or persistence.

In aspects, the invention provides a formulation comprising one or more cyclohexane polyalcohol compound (e.g., scyllo-cyclohexanehexyl compound or epi-cyclohexanehexyl compound) that provides a beneficial pharmacokinetic profile, including but not limited to a sustained pharmacokinetic profile, following treatment.

The invention also provides a formulation intended for administration to a subject to provide a beneficial pharmacokinetic profile, including but not limited to a sustained pharmacokinetic profile, comprising a pure or substantially pure cyclohexane polyalcohol compound, in particular a pure or substantially pure scyllo-cyclohexanehexyl compound or epi-cyclohexanehexyl compound, optionally together with one or more pharmaceutically acceptable carriers, excipients, or vehicles.

The invention also provides a formulation for the treatment of a disorder and/or disease disclosed herein comprising a therapeutically effective amount of a cyclohexane polyalcohol compound, in particular a scyllo-cyclohexanehexyl compound or epi-cyclohexanehexyl compound, to provide a beneficial pharmacokinetic profile, including but not limited to a sustained pharmacokinetic profile, in a pharmaceutically acceptable carrier, excipient, or vehicle.

In an aspect, a formulation comprising a cyclohexane polyalcohol compound, in particular a scyllo-cyclohexanehexyl compound or epi-cyclohexanehexyl compound, is provided which is in a form or which has been adapted for administration to a subject to provide a beneficial pharmacokinetic profile, including but not limited to a sustained pharmacokinetic profile, to treat a disorder and/or disease disclosed herein. In an embodiment, a dosage form is provided such that administration of the dosage form to a subject suffering from a disorder and/or disease disclosed herein provides a beneficial pharmacokinetic profile, including but not limited to a sustained pharmacokinetic profile, resulting in therapeutic effects including without limitation, inhibition, reduction or reversal of one or more of Aβ fibril assembly or aggregation; Aβ toxicity; abnormal protein folding, aggregation, amyloid formation, deposition, accumulation or persistence, and/or amyloid lipid interactions; and, acceleration of disassembly of preformed fibrils, over a dosing period. In particular, the composition is in a form adapted to provide a beneficial pharmacokinetic profile, including but not limited to a sustained pharmacokinetic profile, that results in one or more of the following in a subject for a sustained time over a dosing period: disruption of aggregating Aβ or Aβ oligomers; increased or restored long term potentiation; maintenance of synaptic function; reduced cerebral accumulation of amyloid β; reduced deposition of cerebral amyloid plaques; reduced soluble Aβ oligomers in the brain; reduced glial activity; reduced inflammation, and/or reduced cognitive decline or improvement of cognitive abilities

In an aspect, the invention relates to a dosage form comprising amounts of a cyclohexane polyalcohol compound suitable for administration to a subject to provide effective concentrations, in particular therapeutically effective concentrations, of the compound in an environment of use or an effective dose that results in therapeutic effects in the prevention, treatment, or control of symptoms of a disorder in protein folding and/or aggregation, and/or amyloid formation, deposition, accumulation, or persistence. In aspects, the environment of use is the brain, in particular extracellular or interstitial brain tissue. In other aspects, the environment of use is plasma and/or cerebral spinal fluid (CSF).

In an aspect, the invention relates to a dosage form comprising amounts of a cyclohexane polyalcohol compound suitable for administration to a subject to provide effective concentrations in particular therapeutically effective concentrations, of the compound in plasma, brain and/or cerebral spinal fluid or an effective dose that results in therapeutic effects in the prevention, treatment, or control of symptoms of a disorder in protein folding and/or aggregation, and/or amyloid formation, deposition, accumulation, or persistence. In an aspect, the invention provides a dosage form comprising an amount of a cyclohexane polyalcohol compound suitable for administration to a subject to provide a therapeutically effective concentration of the compound in plasma, brain and/or cerebral spinal fluid or to provide at least one therapeutic effect in the prevention, treatment, or control of symptoms of a disorder in protein folding and/or aggregation, and/or amyloid formation, deposition, accumulation, or persistence.

In another aspect, the invention provides a dosage wherein the therapeutic effects are one or more of inhibition, reduction or reversal in the subject of one or more of Aβ fibril assembly and/or aggregation; Aβ toxicity; abnormal protein folding, abnormal protein aggregation, amyloid formation, deposition, accumulation and/or persistence, amyloid lipid interactions; and acceleration of disassembly of preformed fibrils, over a dosing period. In particular aspects, a dosage form of the invention maintains the compound within an effective plasma or CSF concentration that results in therapeutic effects in the subject.

In another aspect, the invention provides a dosage form comprising an amount of a cyclohexane polyalcohol compound suitable for administration to a subject to provide a therapeutically effective concentration of the compound in plasma, brain and/or cerebral spinal fluid and a pharmaceutically acceptable carrier, diluent or excipient, wherein when the formulation is administered in a dose of 500, 1000, 2000, 3500, 5000 or 7000 mg of said cyclohexane polyalcohol, a mean plasma concentration profile is achieved having a mean AUC_{0-INF} in μg·h/mL of, respectively, 43±20%, 130±20%, 215±20%, 467±20%, 507±20% or 885±20%, and having a mean Cₘₐₓ in μg/mL of, respectively, 5.8±20%, 17±20%, 33±20%, 75±20%, 110±20% or 155±20%.

In another aspect, the present invention is directed to formulations comprising a cyclohexane polyalcohol compound, in particular a scyllo-cyclohexanehexyl compound or epi-cyclohexanehexyl compound, that provides a beneficial pharmacokinetic profile, including but not limited to a sustained pharmacokinetic profile, in the treatment of a disorder and/or disease characterized by amyloid deposition, more particularly Alzheimer's disease.

In an aspect, the invention is directed to a formulation or dosage form suitable for once or twice-a-day administration to treat in a subject a disorder and/or disease disclosed herein comprising one or more cyclohexane polyalcohol compound in an amount effective to provide a beneficial pharmacokinetic profile, including but not limited to a sustained pharmacokinetic profile in the dosing period.

In another aspect, the invention contemplates a dosage form comprising one or more cyclohexane polyalcohol compound, in particular one or more scyllo-cyclohexanehexyl compound or epi-cyclohexanehexyl compound, in an amount effective to maintain the compound within an effective plasma drug concentration that results in therapeutic effects in the subject.

In a further aspect, the invention provides a dosage form comprising one or more cyclohexane polyalcohol compound, in particular one or more scyllo-cyclohexanehexyl compound or epi-cyclohexanehexyl compound, in an amount effective to maintain the compound within an effective CSF drug concentration that results in therapeutic effects in the subject.

In a further aspect, the invention provides a dosage form comprising one or more cyclohexane polyalcohol compound, in particular one or more scyllo-cyclohexanehexyl compound or epi-cyclohexanehexyl compound, in an amount effective to maintain the compound within an effective drug concentration in the brain that results in therapeutic effects in the subject.

In another aspect, the invention relates to a sustained-release dosage form of a cyclohexane polyalcohol compound, which provides a beneficial pharmacokinetic profile.

The release profiles of dosage forms may exhibit different rates and durations of release and may be continuous or pulsatile. Continuous release profiles include release profiles in which a quantity of one or more pharmaceutical compounds is released continuously throughout the dosing interval at either a constant or variable rate. Pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. When two or more components of a dosage form have different release profiles, the release profile of the dosage form as a whole is a combination of the individual release profiles and may be described generally as “multimodal.” The release profile of a two-component dosage form in which each component has a different release profile may described as “bimodal,” and the release profile of a three-component dosage form in which each component has a different release profile may described as “trimodal.” The overall effect of these dosage forms is to provide a substantially sustained release profile because the release profile of the dosage form as a whole is a combination of the individual release profiles.

Similar to the variables applicable to the release profile, the associated plasma profile in a patient may exhibit constant or variable blood plasma concentration levels of the pharmaceutical compounds over the duration of action and may be continuous or pulsatile. Continuous plasma profiles include plasma profiles of all rates and duration which exhibit a single plasma concentration maximum depending on, at least in part, the pharmacokinetics of the pharmaceutical compounds included in the dosage form as well as the release profiles of the individual components of the dosage form, a multimodal release profile may result in either a continuous or a pulsatile plasma profile upon administration to a patient. Preferred release profiles from pulsatile release formulations are those that are substantially continuous release profiles.

The invention also relates to a dosage form of a cyclohexane polyalcohol compound which provides a zero-order or near zero-order release profile.

The invention additionally relates to dosage forms of a cyclohexane polyalcohol compound that provide release profiles that follow mechanisms other than zero order or first order kinetics, for example, but not limited to, square root of time release profiles are also contemplated.

In another aspect the invention relates to dosage forms of a cyclohexane polyalcohol compound that provide release profiles resulting from the combination of any of the release profiles mentioned above.

In another aspect, the invention relates to a dosage form of a cyclohexane polyalcohol compound which provides a zero-order or near zero-order release profile.

The invention additionally relates to a method of preparing a stable formulation or dosage form comprising one or more cyclohexane polyalcohol compound, in particular a scyllo-cyclohexanehexyl compound or epi-cyclohexanehexyl, adapted to provide beneficial pharmacokinetic profiles, in particular sustained pharmacokinetic profiles, following treatment. In a further aspect, the invention provides a method of preparing a stable dosage form comprising mixing an amount of a cyclohexane polyalcohol compound with a pharmaceutically acceptable carrier, excipient or diluent, the mixture being adapted to provide a mean plasma concentration profile characterized by a mean AUC_{0-INF} in μg·h/mL of, respectively, 43±20%, 130±20%, 215±20%, 467±20%, 507±20% or 885±20%, and a mean Cₘₐₓ in μg/mL of, respectively, 5.8±20%, 17±20%, 33±20%, 75±20%, 110±20% or 155±20%. After formulations have been prepared, they can be placed in an appropriate container and labelled for treatment of an indicated condition. For administration of a formulation of the invention, such labelling would include amount, frequency, and method of administration.

In another aspect, the invention provides methods to make commercially available formulations which contain a cyclohexane polyalcohol compound, in particular a scyllo-cyclohexanehexyl compound or epi-cyclohexanehexyl compound, that provides a beneficial pharmacokinetic profile, in particular a sustained pharmacokinetic profile, in the treatment of a disorder and/or disease disclosed herein.

In another aspect, the invention contemplates the use of at least one cyclohexane polyalcohol compound, in particular at least one scyllo-cyclohexanehexyl compound or epi-cyclohexanehexyl compound, for the preparation of a medicament to provide beneficial pharmacokinetic profiles, in particular sustained pharmacokinetic profiles, for preventing and/or treating disorders and/or diseases disclosed herein.

In another aspect, the invention relates to use of at least one cyclohexane polyalcohol compound for the preparation of a medicament to provide, when the medicament is administered in a dose of 500, 1000, 2000, 3500, 5000 or 7000 mg of said cyclohexane polyalcohol, a mean plasma concentration profile having a mean AUC_{0-INF} in μg·h/mL of, respectively, 43±20%, 130±20%, 215±20%, 467±20%, 507±20% or 885±20%, and having a mean Cₘₐₓ in μg/mL of, respectively, 5.8±20%, 17±20%, 33±20%, 75±20%, 110±20% or 155±20%, thereby preventing and/or treating a disorder in protein folding and/or aggregation, and/or amyloid formation, deposition, accumulation, or persistence.

Formulations of the invention may be administered therapeutically or prophylactically to treat disorders and/or diseases disclosed herein, in particular a disorder and/or disease associated with amyloid formation, aggregation or deposition.

Accordingly, the invention provides a method for treating and/or preventing disorders and/or diseases disclosed herein in a subject comprising administering to the subject an effective amount of a formulation or dosage form of the invention.

In a further aspect, the invention also provides a method for treating and/or preventing disorders and/or diseases in a subject comprising administering to the subject one or more, in particular two, dosages of a formulation comprising one or more cyclohexane polyalcohol compound, in particular one or more scyllo-cyclohexanehexyl compound or epi-cyclohexanehexyl compound, in an amount effective to maintain the compound within the effective plasma drug concentration that results in therapeutic effects in the subject.

In a further aspect, the invention provides a method for treating and/or preventing disorders and/or diseases in a subject comprising administering to the subject one or more, in particular two, dosages of a formulation comprising one or more cyclohexane polyalcohol compound, in particular one or more scyllo-cyclohexanehexyl compound or epi-cyclohexanehexyl compound, in an amount effective to maintain the compound within the effective CSF or brain drug concentration that results in therapeutic effects in the subject.

In a further aspect, the invention also provides a method for treating and/or preventing disorders and/or diseases in a subject comprising administering a sustained-release dosage form of one or more cyclohexane polyalcohol compounds, in particular one or more scyllo-cyclohexanehexyl compound or epi-cyclohexanehexyl compounds.

In a further aspect, the invention provides a method for treating and/or preventing disorders and/or diseases in a subject comprising administering a dosage form of one or more cyclohexane polyalcohol compound, in particular one or more scyllo-cyclohexanehexyl compound or epi-cyclohexanehexyl compound, which provides a zero-order or near zero-order release profile. More generally, the invention provides a method for treating Alzheimer's disease in a patient in need thereof comprising administering a dosage form of one or more cyclohexane polyalcohol compound, in particular one or more scyllo-cyclohexanehexyl compound or epi-cyclohexanehexyl compounds, which provide continuous release profiles of either constant or variable rate as well as pulsatile release profiles.

In a further aspect, the invention provides a kit comprising one or more cyclohexane polyalcohol compound, in particular scyllo-cyclohexanehexyl compound or epi-cyclohexanehexyl compound, or a formulation of the invention adapted to provide a beneficial pharmacokinetic profile, in particular a sustained pharmacokinetic profile. In an aspect, the invention provides a kit for preventing and/or treating a disorder and/or disease disclosed herein, comprising a formulation or dosage form of the invention, a container, and instructions for use.

These and other aspects, features, and advantages of the present invention should be apparent to those skilled in the art from the following drawing and detailed description.

### DESCRIPTION OF THE DRAWINGS

The invention will be better understood with reference to the drawings in which:

FIG. 1 is a graph showing the log-plasma concentrations of single doses of 15, 50 and 150 mg/kg a scyllo-cyclohexanehexyl (AZD103) in rats.

FIG. 2 is a graph showing the log-plasma concentrations of single doses of 15, 50 and 150 mg/kg a scyllo-cyclohexanehexyl (AZD103) in dogs.

FIG. 3 is a graph showing log-plasma concentrations of a scyllo-cyclohexanehexyl following oral and intravenous administration of 80 mg/kg in dogs.

FIG. 4 is a graph showing log-plasma concentrations of a scyllo-cyclohexanehexyl following 28 days administration at 15, 50 and 150 mg/kg, twice daily, in rats.

FIG. 5 is a graph showing log-plasma concentrations of a scyllo-cyclohexanehexyl following 14 days administration at 15, 50 and 150 mg/kg, twice daily, in dogs.

FIG. 6 is a graph showing log concentrations of a scyllo-cyclohexanehexyl in plasma and CSF following single oral administration of 240 mg/kg in dogs.

FIG. 7 are graphs showing CSF and brain levels of a scyllo-cyclohexanehexyl and myo-cyclohexanehexyl after ad libitum dosing with a scyllo-cyclohexanehexyl or myo-cyclohexanehexyl for one month, and in untreated animals.

FIG. 8 shows representative traces of GCMS analysis detecting inositol constituents of phosphatidylinositol lipids from the brains of mice that had received ad libitum administration of a scyllo-cyclohexanehexyl for one month, and in untreated animals.

FIG. 9 is a graph showing the dose response effect of a scyllo-cyclohexanehexyl on cognitive performance of TgCRND8 mice. The indicated dose levels were administered to mice from 3-4 months of age.

FIG. 10: Aβ-dependent cognitive impairment is therapeutically alleviated by a scyllo-cyclohexanehexyl. Swim path length in the Morris Water Maze test was evaluated in transgenic (Tg) and non-transgenic (nTg) animals, receiving the indicated treatments between 5 and 6 months of age. Animals were assessed at 6 months.

FIG. 11: Scyllo-cyclohexanehexyl dose response in rescue of cognitive impairment and reduction of plaque burden. Mice were treated from 12 to 16 weeks of age. For swim path length in the Morris Water Maze, ad libitum dosing data was historic.

FIG. 12: Scyllo-cyclohexanehexyl dose response confirmation: amyloid reduction. TgCRND8 mice were treated between 5-6 months with the indicated dose levels of a scyllo-cyclohexanehexyl.

FIG. 13: scyllo-Inositol treatment effectively reduces TgCRND8 plaque levels with no preference for plaque size. TgCRND8 mice were given 2-months of scyllo-inositol treatment starting at 5 months of age. (a) The percent brain area covered in plaques was significantly reduced in the scyllo-inositol treated animals compared to the control group. *=p<0.0001. (b) scyllo-Inositol treatment (grey bars) reduced the number of plaques observed, regardless of plaque size, when compared to control animals (black bars). Plaques were categorized as being either <100, 100-250, 250-500 or >500 μm² insize.

FIG. 14: Myo- and scyllo-inositol concentrations in CSF (a) and brain (b) of untreated or treated with myo-inositol or scyllo-inositol ad libitum. D-chiro-inositol was used as an internal standard for the GC/MS assay. (a) Ad libitum myo-inositol treatment did not significantly change either myo-inositol (black bars) or scyllo-inositol (grey bars) levels in the CSF, however, scyllo-inositol treatment significantly increased CSF scyllo-inositol. (b) Ad libitum myo-inositol treatment significantly decreased scyllo-inositol levels in the brain compared to the untreated group. In contrast, scyllo-inositol treatment significantly increased brain scyllo-inositol levels. *, [symbol] =p<0.001 compared to the untreated group. (n³ 5 animals per treatment).

FIG. 15: scyllo-Inositol concentration in CSF of untreated, ad libitum or once-daily scyllo-inositol treated mice. The once-daily treatment was at 10 mg/kg, 30 mg/Kg or 100 mg/kg scyllo-inositol by gavage and mice were sacrificed 8 h following the last treatment. Ad libitum treatment resulted in a significant increase in scyllo-inositol concentration in both the CSF and brain when compared to all other groups. *=p<0.001 compared to all other groups. (n=4 per treatment).

FIG. 16. Bioavailability of scyllo- (solid line) and myo-inositol (dashed line) in plasma and brain, determined using orally administered tritiated-inositol uptake studies. Plasma levels of myo- and scyllo-inositol increased rapidly peaking at 2 h and 12 h post administration, respectively. Brain levels also rose rapidly and were maximal at 8 h and 32 h, respectively.

FIG. 17. A competition assay with myo-inositol to compete scyllo-inositol uptake, following a single oral gavage dose was examined. (A) Plasma 3^{H}-scyllo-inositol levels following co-administration of 0, 50, 200 or 400 μg of myo-inositol. Myo-inositol loading appears to alter the kinetics of oral scyllo-inositol uptake in a dose-dependent manner. (B) Brain levels of scyllo-inositol at 4 h following myo-inositol administration. Scyllo-inositol levels are not significantly changed following myo-inositol dosing.

FIG. 18. GC/MS. Derivatization and detection of myo-, scyllo- and chiro-inositols.

FIG. 19. Scyllo-inositol concentration in the brain and CSF of untreated, ad libitum or once daily scyllo-inositol treated mice. The once-daily treatment was a gavage dose of either 10 mg/kg, 30 mg/Kg or 100 mg/kg scyllo-inositol and mice were sacrificed 8 h following last treatment. Ad libitum treatment resulted in a significant increase in scyllo-inositol levels in both the CSF and brain when compared to all other groups. *=p<0.001 compared to all other groups.

FIG. 20. GC/MS profiles of myo- and scyllo-inositol isolated from phosphatidylinositol in untreated (A) versus scyllo-inositol treated mice (B). The inositol compounds were derivatized, chiro-inositol was added as an internal standard and single mass ion m/z 168 was used to quantify inositol. Myo-inositol was readily detected but scyllo-inositol could not be detected in any of the samples.

FIG. 21 is a graph showing mean concentration-time profiles for a phase 1 single ascending dose, double-blind, randomized, placebo-controlled study to evaluate oral doses of AZD-103 in healthy male volunteers.

FIG. 22 is a graph showing mean log concentration-time profiles for a phase 1 single ascending dose, double-blind, randomized, placebo-controlled study to evaluate oral doses of AZD-103 in healthy male volunteers.

### DETAILED DESCRIPTION OF EMBODIMENTS

### Glossary

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

The terms “administering” and “administration” refer to the process by which a therapeutically effective amount of a formulation or dosage form contemplated herein is delivered to a subject for treatment, including prevention, purposes. Compositions and formulations are administered in accordance with good medical practices taking into account the subject's clinical condition, age, sex, body weight, and other factors known to physicians.

The term “treating” refers to reversing, alleviating, or inhibiting the progress of a disorder and/or disease disclosed herein, or one or more symptoms of such disorder and/or disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a formulation or dosage form of the present invention to a subject that is not at the time of administration afflicted with the disease. “Preventing” also refers to preventing the recurrence of a disease or of one or more symptoms associated with such disease. The terms “treatment” and “therapeutically,” refer to the act of treating, as “treating” is defined above. The terms “treating” and “preventing” may also be used independently herein to refer to reversing, alleviating or inhibiting the progress or symptoms of a disorder and/or disease, or preventing the onset or symptoms of a disease, respectively.

The terms “subject”, “individual”, or “patient” are used interchangeably herein and refer to an animal including a warm-blooded animal such as a mammal, which is afflicted with or suspected of having or being pre-disposed to a disorder and/or disease disclosed herein. Mammal includes without limitation any members of the Mammalia. In aspects of the invention, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. Typical subjects for treatment include persons susceptible to, suffering from or that have suffered a disorder and/or disease disclosed herein. A subject may or may not have a genetic predisposition for a disorder and/or disease disclosed herein such as Alzheimer's disease. In embodiments of the invention the subjects are susceptible to, or suffer from Alzheimer's disease. In some aspects, a subject shows signs of cognitive deficits and amyloid plaque neuropathology.

The term “beneficial pharmacokinetic profile” refers to levels of a cyclohexane polyalcohol compound in plasma and/or cerebral spinal fluid, amounts or doses of a cyclohexane polyalcohol compound that provide levels of the compound in plasma and/or cerebral spinal fluid, or a required dose, that results in therapeutic effects in the prevention, treatment, or control of symptoms of a disease and/or condition disclosed herein. The term “sustained pharmacokinetic profile” as used herein refers to a length of time over which efficacious levels of a biologically active cyclohexane polyalcohol compound are in its environment of use. It is preferable that the sustained pharmacokinetic profile be such that a single or twice daily administration, preferably twice daily administration, adequately prevents, treats, or controls symptoms of a disease and/or condition disclosed herein. It is also preferable that efficacious levels of the compound remain in the plasma brain, and/or CSF from about 12 hours to about 36 hours, more preferably 12 hours to about 24 hours, and most preferably from about 20 hours to about 24 hours.

A “therapeutic effect” refers to an effect of a formulation, dosage form, drug delivery technology or method disclosed herein, including improved biological activity and efficacy. A therapeutic effect may be a sustained therapeutic effect that correlates with a substantially constant plasma, brain and/or CSF concentration of a cyclohexane polyalcohol compound over a dosing period, in particular a sustained dosing period. A therapeutic effect may be a statistically significant effect in terms of statistical analysis of an effect of a cyclohexane polyalcohol compound, in particular a scyllo-cyclohexanehexyl compound or epi-cyclohexanehexyl, versus the effects without the compound. “Statistically significant” or “significantly different” effects or levels may represent levels that are higher or lower than a standard. In embodiments of the invention, the difference may be 1.5, 2, 3, 4, 5, 6, 7, 8, 9, 10, 15, 20, 25 or 50 times higher or lower compared with the effect obtained without a cyclohexane polyalcohol compound.

In an embodiment, where the disease is Alzheimer's disease, therapeutic effects of a formulation, dosage form or method of the invention can manifest as at least one, two, three, four, five, six, seven, eight, nine, ten, twelve, thirteen, fourteen, fifteen, or all of the following, in particular five or ten or more, more particularly fifteen or more of the following:
a) Prevention, increase or restoration of long term potentiation relative to the level in the absence of a formulation or dosage form disclosed herein after administration to a subject with symptoms of Alzheimer's disease. In aspects of the invention a formulation or dosage form induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in long term potentiation in a subject.
b) Prevention, increase or maintenance of synaptic function relative to the level of synaptic function in the absence of a formulation or dosage form disclosed herein after administration to a subject with symptoms of Alzheimer's disease. In aspects of the invention a formulation or dosage form induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, 99%, 100%, 125%, 150%, 175% or 200% increase in synaptic function in a subject.
c) An increase in synaptophysin. In aspects of the invention there is at least about a 2%, 5%, 10%, 15%, 20%, 30%, 40%, 50%, 60%, 70%, 80%, 90%, 95%, 99%, 100%, 125%, 150%, 175% or 200% increase in synaptophysin.
d) An increase in synaptophysin reactive boutons and cell bodies. In aspects of the invention there is at least about a 2%, 5%, 10%, 15%, 20%, 30%, 40%, 50%, 60%, 70%, 80%, 90%, 95%, 99%, 100%, 125%, 150%, 175% or 200%, more particularly about a 100-150% or 140-150%, increase in synaptophysin reactive boutons and cell bodies.
e) Prevention, reduction or an absence of symptoms of inflammation, in particular an Aβ induced inflammatory response, after administration to a subject with symptoms of Alzheimer's disease.
f) Prevention, reduction in cerebral accumulation of Aβ relative to the levels measured in the absence of a formulation or dosage form of the invention in subjects with symptoms of Alzheimer's disease. In aspects of the invention, a formulation or dosage form induces at least about a 2%, 5%, 10%, 15%, 20%, 30%, 40%, 50%, 60%, 70%, 80%, or 90% decrease in cerebral accumulation of Aβ.
g) Prevention, reduction in deposition of cerebral amyloid plaques, relative to the levels measured in the absence of a formulation or dosage form of the invention in subjects with symptoms of Alzheimer's disease. In aspects of the invention, a formulation or dosage form induces at least about a 2%, 5%, 10%, 15%, 20%, 30%, 40%, 50%, 60%, 70%, 80%, or 90% decrease in deposition of cerebral amyloid plaques.
h) A reduction in plaque number. In aspects of the invention, a formulation or dosage form of the invention induces at least about a 2%, 5%, 10%, 15%, 20%, 30%, 40%, 50%, 60%, 70%, 80%, or 90% reduction in plaque number. In particular aspects a formulation or dosage form induces a 5-15% or 10-15% reduction in plaque number.
i) A reduction in plaque size. In aspects of the invention, a formulation or dosage form of the invention induces at least about a 2%, 5%, 10%, 15%, 20%, 30%, 40%, 50%, 60%, 70%, 80%, or 90% reduction in plaque size. In particular aspects a formulation or dosage form of the invention induces a 5-15% or 10-15% reduction in plaque size.
j) A reduction in percent area of the brain covered in plaques. In aspects of the invention, a formulation or dosage form of the invention induces at least about a 2%, 5%, 10%, 15%, 20%, 30%, 40%, 50%, 60%, 70%, 80%, or 90% reduction in percent area of the brain covered in plaques. In particular aspects a formulation or dosage form of the invention induces a 5-15% or 10-15% reduction in percent area of the brain covered in plaques.
k) A reduction in soluble Aβ oligomers in the brain, relative to the levels measured in the absence of a formulation or dosage form of the invention in subjects with symptoms of Alzheimer's disease. In aspects of the invention, a formulation or dosage form of the invention induces at least about a 2%, 5%, 10%, 15%, 20%, 30%, 40%, 50%, 60%, 70%, 80%, or 90% decrease in soluble Aβ oligomers.
l) A reduction in brain levels of Aβ 40. In aspects of the invention, a formulation or dosage form of the invention induces at least about a 2%, 5%, 10%, 15%, 20%, 30%, 40%, 50%, 60%, 70%, 80%, or 90% reduction in Aβ 40. In particular aspects a formulation or dosage form of the invention induces a 10-50%, 20-45%, or 25-35% reduction in brain levels of Aβ 40.
m) A reduction in brain levels of Aβ 42. In aspects of the invention, a formulation or dosage form of the invention induces at least about a 2%, 5%, 10%, 15%, 20%, 30%, 40%, 50%, 60%, 70%, 80%, or 90% reduction in Aβ 42. In particular aspects a formulation or dosage form of the invention induces a 10-50%, 15-40%, or 20-25% reduction in brain levels of Aβ 42.
n) A reduction in glial activity in the brain, relative to the levels measured in the absence of a formulation or dosage form of the invention in subjects with symptoms of Alzheimer's disease. Preferably, a formulation or dosage form induces at least about a 2%, 5%, 10%, 15%, 20%, 30%, 40%, 50%, 60%, 70%, 80%, or 90% decrease in glial activity
o) Maintenance of synaptic function at about normal for a prolonged period of time, in particular for at least 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 24 months following treatment.
p) A reduction or slowing of the rate of disease progression in a subject with Alzheimer's disease. In particular a reduction or slowing of cognitive decline in a subject with Alzheimer's disease.
q) Prevention, reduction or slowing of cognitive deficits or improvement of cognitive abilities.
r) Prevention, reduction or slowing of amyloid angiopathy.
s) A reduction in accelerated mortality.
t) An increase in survival in a subject with symptoms of Alzheimer's disease.

In aspects of the invention therapeutic effects of a formulation, dosage form or treatment of the invention can manifest as (a) and (b); (a), (b) and (c); (a), (b), (e), (f) and (g); (a), (b), (e), (f) through (h); (a), (b), (e), (f) through (i); (a), (b), (e), (f) through (j); (a), (b), (e), (f) through (k); (a), (b), (e), (f) through (l); (a), (b), (e), (f) through (m); (a), (b), (e), (f) through (n); (a), (b), (e), (f) through (o); (a), (b), (e), (f) through (p); (a), (b), (e), (f) through (q); (a), (b), (e), (f) through (r); (a), (b), (e), (f) through (s); (a), (b), (e), (f) through (t); (a) through (d); (a) through (e); (a) through (f); (a) through (g); (a) through (h); (a) through (i); (a) through (j); (a) through (k); (a) through (l); (a) through (m); (a) through (n); (a) through (o); (a) through (p); (a) through (q); (a) through (r); (a) through (s); and (a) through (t).

“Therapeutically effective amount” relates to the amount or dose of a cyclohexane polyalcohol compound in a formulation or dosage form that will provide or lead to a beneficial pharmacokinetic profile, more particularly a sustained pharmacokinetic profile.

A “therapeutically effective concentration” refers to levels of a cyclohexane polyalcohol compound in plasma, brain and/or cerebral spinal fluid to provide a beneficial pharmacokinetic profile, more particularly a sustained pharmacokinetic profile, or at least one therapeutic effect.

The term “pure” in general means better than 90%, 92%, 95%, 97%, 98% or 99% pure, and “substantially pure” means a compound synthesized such that the compound, as made as available for consideration into a formulation or dosage form of the invention, has only those impurities that can not readily nor reasonably be removed by conventional purification processes.

A “cyclohexane polyalcohol compound” is understood to refer to any compound, which fully or partially, directly or indirectly, provides one or more beneficial effects described herein and includes a compound of the formula I, II, III or IV described herein, or an analog or derivative thereof. In aspects of the invention, the cyclohexane polyalcohol compound is an inositol.

A cyclohexane polyalcohol compound includes a pharmaceutically acceptable salt. “Pharmaceutically acceptable salt(s),” means a salt that is pharmaceutically acceptable and has the desired pharmacokinetic properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

A cyclohexane polyalcohol compound includes a functional derivative. A “functional derivative” refers to a compound that possesses a biological activity (either functional or structural) that is substantially similar to the biological activity of a compound disclosed herein. The term “functional derivative” is intended to include “variants” “analogs” or “chemical derivatives” of a cyclohexane polyalcohol compound. The term “variant” is meant to refer to a molecule substantially similar in structure and function to a cyclohexane polyalcohol compound or a part thereof. A molecule is “substantially similar” to a cyclohexane polyalcohol compound If both molecules have substantially similar structures or if both molecules possess similar biological activity. The term “analog” refers to a molecule substantially similar in function to a cyclohexane polyalcohol compound. The term “chemical derivative” describes a molecule that contains additional chemical moieties which are not normally a part of the base molecule.

A cyclohexane polyalcohol compound includes crystalline forms which may exist as polymorphs. Solvates of the compounds formed with water or common organic solvents are also intended to be encompassed within the term. In addition, hydrate forms of the compounds and their salts are encompassed within this invention. Further prodrugs of compounds of cyclohexane polyalcohol compounds are encompassed within the term.

The term “solvate” means a physical association of a compound with one or more solvent molecules or a complex of variable stoichiometry formed by a solute (for example, a compound of the invention) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like. Dehydrate, co-crystals, anhydrous, or amorphous forms of the compounds of the invention are also included. The term “hydrate” means a solvate wherein the solvent molecule(s) is/are H₂O, including, mono-, di-, and various poly-hydrates thereof. Solvates can be formed using various methods known in the art.

Crystalline compounds of the invention can be in the form of a free base, a salt, or a co-crystal. Free base compounds can be crystallized in the presence of an appropriate solvent in order to form a solvate. Acid salt compounds of the invention (e.g. HCl, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a compounds of the invention would have about 1 equivalent of solvent (H₂O) for each equivalent of a compound of the invention. However, more or less solvent may be used depending on the choice of solvate desired.

The compounds of the invention may be amorphous or may have different crystalline polymorphs, possibly existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987, each of which is incorporated herein by reference in their entireties.

Examples of prodrugs include, but are not limited to esters (e.g., acetate, formate, and benzoate derivatives), carbamates (e.g. N,N-dimethylaminocarbonyl) of hydroxy functional groups on compounds of the present invention, and the like

In general, all physical forms are intended to be within the scope of the present invention.

In aspects of the invention, the cyclohexane polyalcohol compound includes a compound with the base structure of the formula I, in particular a substantially pure, compound of the formula I.

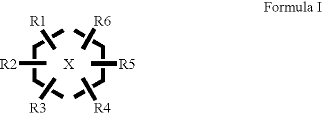

wherein X is a cyclohexane, in particular a myo-, scyllo-, epi-, chiro, or allo-inositol radical, wherein one or more of R¹, R², R³, R⁴, R⁵, and R⁶ are independently hydroxyl, alkyl, alkenyl, alkynyl, alkylene, alkenylene, alkoxy, alkenyloxy, cycloalkyl, cycloalkenyl, cycloalkoxy, cycloalkynyl, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and a pharmaceutically acceptable salt, isomer, solvate, or prodrug thereof. In aspects of the invention, four or five or all of R¹, R², R³, R⁴, R⁵, and/or R⁶ are hydroxyl. In particular aspects of the invention, a cyclohexane polyalcohol compound of the formula I is used wherein X is a radical of scyllo-inositol or epi-inositol.

Aspects of the invention use classes of cyclohexane polyalcohol compounds of the formula II, in particular isolated and pure, in particular substantially pure, compounds of the formula II:

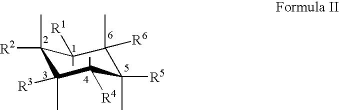

wherein R¹, R², R³, R⁴, R⁵, and R⁶ are hydroxyl, or one or more of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ are hydroxyl, or a pharmaceutically acceptable salt thereof.

In aspects of the invention, the cyclohexane polyalcohol compound is a substantially pure, compound of the formula I or II as defined herein with the proviso that when (a) one of R¹, R², R³, R⁴, R⁵, and/or R⁶ are alkyl or fluorine no more than four of the other of R¹, R², R³, R⁴, R⁵, and/or R⁶ are hydroxyl, (b) one of R¹, R², R³, R⁴, R⁵, and/or R⁶ is amino or azide no more than four of R¹, R², R³, R⁴, R⁵, and R⁶ are hydroxyl, (c) two of R¹, R², R³, R⁴, R⁵, and/or R⁶ are amino, no more than three of R¹, R², R³, R⁴, R⁵, and R⁶ are hydroxyl, and (d) three of R¹, R², R³, R⁴, R⁵, and/or R⁶ are amino, carboxyl, carbamyl, sulfonyl, isoxasolyl, imidazolyl, or thiazolyl, the other of R¹, R², R³, R⁴, R⁵, and/or R⁶ cannot all be hydroxyl. In aspects of the invention, the cyclohexane polyalcohol compound is a substantially pure, compound of the formula III,

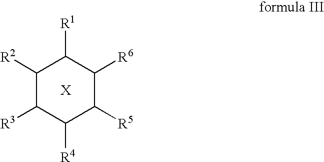

wherein X is a cyclohexane ring, where R¹, R², R³, R⁴, R⁵, and R⁶ are hydroxyl, or at least one of R¹, R², R³, R⁴, R⁵, and R⁶ is independently selected from hydrogen, C₁-C₆ alkyl, C₂-C₆ alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷, —NR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀ aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof. In particular aspects the invention utilizes isomers of the compound of the formula III, more particularly scyllo- or epi-isomers.

In aspects of the invention, the cyclohexane polyalcohol compound is a substantially pure, compound of the formula IV,

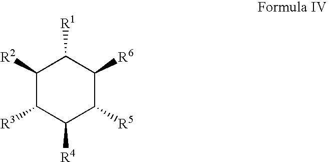

wherein R¹, R², R³, R⁴, R⁵, and R⁶ are defined as for formula III, or a pharmaceutically acceptable salt thereof.

The terms used herein for radicals including “alkyl”, “alkoxy”, “alkenyl”, “alkynyl”, “hydroxyl” etc, refer to both unsubstituted and substituted radicals. The term “substituted,” as used herein, means that any one or more moiety on a designated atom (e.g., hydroxyl) is replaced with a selected group provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

“Alkyl”, either alone or within other terms such as “arylalkyl” means a monovalent, saturated hydrocarbon radical which may be a straight chain (i.e. linear) or a branched chain. In certain aspects of the invention, an alkyl radical comprises from about 1 to 24 or 1 to 20 carbon atoms, preferably from about 1 to 10, 1 to 8, 3 to 8, 1 to 6, or 1 to 3-carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents at positions that do not significantly interfere with the preparation of the cyclohexane polyalcohol compounds and do not significantly reduce the efficacy of the compounds. An alkyl radical may be optionally substituted. In certain aspects, an alkyl radical is substituted with one to five substituents including halo, lower alkoxy, haloalkoxy, alkylalkoxy, haloalkoxyalkyl, hydroxyl, cyano, nitro, thio, amino, substituted amino, carboxyl, sulfonyl, sulfenyl, sulfinyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, aryl (e.g., phenylmethyl (i.e. benzyl)), heteroaryl (e.g., pyridyl), and heterocyclic (e.g., piperidinyl, morpholinyl).

In aspects of the invention, “substituted alkyl” refers to an alkyl group substituted by, for example, one to five substituents, and preferably 1 to 3 substituents, such as alkyl, alkoxy, oxo, alkanoyl, aryl, aralkyl, aryloxy, alkanoyloxy, cycloalkyl, acyl, amino, hydroxyamino, alkylamino, arylamino, alkoxyamino, aralkylamino, cyano, halogen, hydroxyl, carboxyl, carbamyl, carboxylalkyl, keto, thioketo, thiol, alkylthiol, arylthio, aralkylthio, sulfonamide, thioalkoxy, and nitro.

The term “alkenyl” refers to an unsaturated, acyclic branched or straight-chain hydrocarbon radical comprising at least one double bond. Alkenyl radicals may contain from about 2 to 24 or 2 to 10 carbon atoms, preferably from about 3 to 8 carbon atoms and more preferably about 3 to 6 or 2 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. Preferred alkenyl groups include ethenyl (—CH═CH₂), n-propenyl (—CH₂CH═CH₂), iso-propenyl (—C(CH₃)═CH₂), and the like. An alkenyl radical may be optionally substituted similar to alkyl.

In aspects of the invention, “substituted alkenyl” refers to an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkynyl” refers to an unsaturated, branched or straight-chain hydrocarbon radical comprising one or more triple bonds. Alkynyl radicals may contain about 1 to 20, 1 to 15, or 2-10 carbon atoms, preferably about 3 to 8 carbon atoms and more preferably about 3 to 6 carbon atoms.

In aspects of the invention, “alkynyl” refers to straight or branched chain hydrocarbon groups of 2 to 6 carbon atoms having one to four triple bonds. Examples of suitable alkynyl radicals include ethynyl, propynyls, such as prop-1-yn-1-yl, prop-2-yn-1-yl, butynyls such as but-1-yn-1-yl, but-1-yn-3-yl, and but-3-yn-1-yl, pentynyls such as pentyn-1-yl, pentyn-2-yl, and 4-methoxypentyn-2-yl, and 3-methylbutyn-1-yl, hexynyls such as hexyn-1-yl, hexyn-2-yl, and hexyn-3-yl, and 3,3-dimethylbutyn-1-yl radicals and the like. This radical may be optionally substituted similar to alkyl. The term “cycloalkynyl” refers to cyclic alkynyl groups.

In aspects of the invention, “substituted alkynyl” refers to an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

The term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, ethylidene, methylethylene, and isopropylidene.

The term “alkenylene” refers to a linear or branched radical having from about 2 to 10, 2 to 8 or 2 to 6 carbon atoms, at least one double bond, and having attachment points for two or more covalent bonds.

Examples of such radicals are 1,1-vinylidene (CH₂═C), 1,2-vinylidene (—CH═CH—), and 1,4-butadienyl (—CH═CH—CH═CH—).

As used herein, “halogen” or “halo” refers to fluoro, chloro, bromo and iodo, especially fluoro or chloro.

The term “hydroxyl” or “hydroxy” refers to a single —OH group. The term “cyano” refers to a carbon radical having three of four covalent bonds shared by a nitrogen atom, in particular —CN.

The term “alkoxy” refers to a linear or branched oxy-containing radical having an alkyl portion of one to about ten carbon atoms, which may be substituted. Particular alkoxy radicals are “lower alkoxy” radicals having about 1 to 6, 1 to 4 or 1 to 3 carbon atoms. An alkoxy having about 1-6 carbon atoms includes a C₁-C₆ alkyl-O— radical wherein C₁-C₆ alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g., alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

In aspects of the invention, “acyl” refers to a group —C(O)R¹⁰, where R¹⁰ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

The term “cycloalkyl” refers to radicals having from about 3 to 16 or 3 to 15 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃-C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted.

In aspects of the invention, “substituted cycloalkyl” refers to cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

The term “cycloalkenyl” refers to radicals comprising about 2 to 16, 4 to 16, 2 to 15, 2 to 10, 4 to 10, 3 to 8, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms, in particular cyclobutenyl, cyclopentenyl, cyclohexenyl and cycloheptenyl. A cycloalkenyl radical may be optionally substituted with groups as disclosed herein.

The term “cycloalkoxy” refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. In aspects of the invention an aryl radical comprises 4 to 24 carbon atoms, in particular 4 to 10, 4 to 8, or 4 to 6 carbon atoms. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, diphenyl, acephthylenyl, fluorenyl, phenalenyl, phenanthrenyl, and anthracenyl, preferably phenyl. An aryl radical may be optionally substituted with one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenyzl, and amino benzyl.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy” as used herein, refers to an aryl group attached to an alkoxy group. Representative examples of arylalkoxy include, but are not limited to, 2-phenylethoxy, 3-naphth-2-ylpropoxy, and 5-phenylpentyloxy.

The term “aroyl” refers to aryl radicals, as defined above, attached to a carbonyl radical as defined herein, including without limitation benzoyl and toluoyl. An aroyl radical may be optionally substituted with groups as disclosed herein.

The term “heteroaryl” refers to fully unsaturated heteroatom-containing ring-shaped aromatic radicals having from 3 to 15, 3 to 10, 5 to 15, 5 to 10, or 5 to 8 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed herein.

The term “heterocyclic” refers to saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitation a saturated 3 to 6-membered heteromonocylic group containing 1 to 4 nitrogen atoms [e.g. pyrrolidinyl, imidazolidinyl, piperidinyl, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like.

The term “sulfate”, used alone or linked to other terms, is art recognized and includes a group that can be represented by the formula:

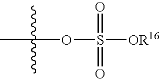

wherein R¹⁶ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfonyl”, used alone or linked to other terms such as alkylsulfonyl or arylsulfonyl, refers to the divalent radicals —SO₂—. In aspects of the invention where one or more of R¹, R³, R⁴, R⁵, or R⁶ is a sulfonyl group, the sulfonyl group may be attached to a substituted or unsubstituted alkyl, alkenyl, alkynyl, aryl, cycloalkyl, cycloalkenyl, cycloalkynyl, or heterocyclic group, carbohydrate, peptide, or peptide derivative.

The term “sulfonate” is art recognized and includes a group represented by the formula:

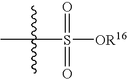

wherein R¹⁶ is an electron pair, hydrogen, alkyl, cycloalkyl, aryl, alkenyl, alkynyl, cycloalkenyl, cycloalkynyl, heterocyclic, carbohydrate, peptide, or peptide derivative

Examples of sulfonated alkyl groups include ethyl sulfuric acid, ethanesulfonic acid, 2-aminoethan-1-ol sulfuric acid, 1-propanesulfonic acid, 2-propanesulfonic acid, 1,2-diethanedisulfonic acid, 1,2-ethanediol disulfuric acid, 1,3-propanedisulfonic acid, 1-propanol sulfuric acid, 1,3-propanediol disulfuric acid, 1-butanesulfonic acid, 1,4-butanediol disulfuric acid, 1,2-ethanediol disulfuric acid, 3-amino-1-propanesulfonic acid, 3-hydroxypropanesulfonic acid sulfate, 1,4-butanesulfonic acid, 1,4-butanediol monosulfuric acid, 1-pentanesulfonic acid, 1,5-pentanedisulfonic acid, 1,5-pentanediol sulfuric acid, 4-heptanesulfonic acid, 1,3,5-heptanetriol trisulfate, 2-hydroxymethyl-1,3-propanediol trisulfate, 2-hydroxymethyl-2-methyl-1,3-propanediol trisulfate, 1,3,5,7-heptanetetraol tetrasulfate, 1,3,5,7,9-nonane pentasulfate, 1-decanesulfonic acid, and pharmaceutically acceptable salts thereof.

Examples of cycloalkyl sulfonated groups include 1,3-cyclohexanediol disulfate, and 1,3,5-heptanetriol trisulfate.

Examples of aryl sulfonated groups include 1,3-benzenedisulfonic acid, 2,5-dimethoxy-1,4-benzenedisulfonic acid, 4-amino-3-hydroxy-1-naphthalenesulfonic acid, 3,4-diamino-1-naphthalenesulfonic acid, and pharmaceutically acceptable salts thereof.

Examples of heterocyclic sulfonated compounds include 3-(N-morpholino)propanesulfonic acid and tetrahydrothiophene-1,1-dioxide-3,4-disulfonic acid, and pharmaceutically acceptable salts thereof.

Examples of sulfonated carbohydrates are sucrose octasulfonate, 5-deoxy-1,2-O-isopropylidene-α-D-xylofuranose-5-sulfonic acid or an alkali earth metal salt thereof, methyl-α-D-glucopyranoside 2,3-disulfate, methyl 4, —O-benzylidene-α-D-glucopyranoside 2,3-disulfate, 2,3,4,3′,4′-sucrose pentasulfate, 1,3:4,6-di-O-benzylidene-D-mannitol 2,5-disulfate, D-mannitol 2,5-disulfate, 2,5-di-O-benzyl-D-mannitol tetrasulfate, and pharmaceutically acceptable salts thereof.

The term “sulfonyl”, used alone or linked to other terms such as alkylsulfinyl (i.e. —S(O)-alkyl) or arylsulfinyl, refers to the divalent radicals —S(O)—.

The term “sulfoxide” refers to the radical —S═O.

The term “amino”, alone or in combination, refers to a radical where a nitrogen atom (N) is bonded to three substituents being any combination of hydrogen, hydroxyl, alkyl, cycloalkyl, alkenyl, alkynyl, aryl or silyl with the general chemical formula —NR¹⁰R¹¹ where R¹⁰ and R¹¹ can be any combination of hydrogen, hydroxyl, alkyl, cycloalkyl, alkenyl, alkynyl, aryl, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidine, benzylamino, diphenylmethylamino, tritylamino, trimethylsilylamino, and dimethyl-tert.-butylsilylamino.

The term “thiol” means —SH.

The term “sulfenyl” refers to the radical —SR⁹ wherein R⁹ is not hydrogen. R⁹ may be alkyl, alkenyl, alkynyl, cycloalkyl, aryl, silyl, heterocyclic, heteroaryl carbonyl, or carboxyl.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR¹² where R¹² is an aryl group which may be substituted. Illustrative examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “thioalkoxy”, alone or in combination, refers to a chemical functional group where a sulfur atom (S) is bonded to an alkoxy group with the general chemical formula —SR¹³ where R¹³ is an alkoxy group which may be substituted. In aspects of the invention a “thioalkoxy group” has 1-6 carbon atoms and refers to a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

The term “carbonyl” refers to a carbon radical having two of the four covalent bonds shared with an oxygen atom.

The term “carboxyl”, alone or in combination, refers to —C(O)OR¹⁴— wherein R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted. In aspects of the invention, the carboxyl groups are in an esterified form and may contain as an esterifying group lower alkyl groups. In particular aspects of the invention, —C(O)OR¹⁴ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amine, halo, alkylamino, aryl, carboxyl, or a heterocyclic. In particular aspects of the invention, the carboxyl group is methoxycarbonyl, butoxycarbonyl, tert alkoxycarbonyl such as tert.butoxycarbonyl, arylmethyoxycarbonyl having one or two aryl radicals including without limitation phenyl optionally substituted by, for example, lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyl and dimethyltert.butylsilyl.

The term “carboxamide”, alone or in combination, refers to amino, monoalkylamino, dialkylamino, monocycloalkylamino, alkylcycloalkylamino, and dicycloalkylamino radicals, attached to one of two unshared bonds in a carbonyl group.

The term “nitro” means —NO₂—.

A radical in a cyclohexane polyalcohol compound may be substituted with one or more substituents apparent to a person skilled in the art including without limitation alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, sulfenyl, alkylsulfonyl, sulfinyl, alkylsulfinyl, aralkyl, heteroaralkyl, cycloalkyl, cycloalkenyl, cycloalkoxy, cycloalkenyloxy, amino, oxy, halo, azido, thio, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfonyl, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. In embodiments of the invention, the substituents include alkyl, alkoxy, alkynyl, halo, amino, thio, oxy, and hydroxyl.

While broad definitions of cyclohexane polyalcohol compounds are described herein for use in the present invention, certain compounds of formula I, II, III or IV may be more particularly described.

In embodiments of the invention, the cyclohexane polyalcohol compound is an isolated, in particular pure, more particularly substantially pure, compound of the formula I, wherein X is a radical of scyllo-inositol, epi-inositol or a configuration isomer thereof, wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In aspects of the invention, the cyclohexane polyalcohol compound is a scyllo-cyclohexanehexyl compound, in particular pure or substantially pure scyllo-inositol. The compound “scyllo-inositol” is also referred to herein as AZD-103 or ELND005.

A “scyllo-cyclohexanehexyl compound” includes compounds having the structure of the formula Va or Vb:

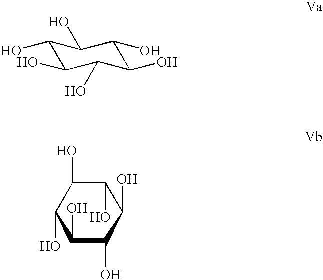

In embodiments, a scyllo-cyclohexanehexyl compound, salt, or derivative thereof, in particular a pure or substantially pure scyllo-cyclohexanehexyl compound, is used in the formulations, dosage forms, methods and uses disclosed herein. A scyllo-cyclohexanehexyl compound includes a compound of the formula Va or Vb wherein one, two, three or four, preferably one, two or three, more preferably one or two hydroxyl groups are replaced by substituents, in particular univalent substituents, with retention of configuration. Suitable substituents include without limitation hydrogen, alkyl, acyl, alkenyl, cycloalkyl, halogen, —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; and —OR³ wherein R³ is hydrogen, alkyl, or —SO₃H. In aspects of the invention, a scyllo-cyclohexanehexyl compound does not include scyllo-cyclohexanehexyl substituted with one or more phosphate group.

Particular aspects of the invention utilize scyllo-cyclohexanehexyl compounds of the formula Va or Vb wherein one or more of the hydroxyl groups is replaced with alkyl, acyl, alkenyl, —NHR¹ wherein R¹ is hydrogen, acyl, alkyl or —R²R³ wherein R² and R³ are the same or different and represent acyl or alkyl; —SR⁴ wherein R⁴ is hydrogen, alkyl, or —O₃H; and —OR³ wherein R³ is hydrogen, alkyl, or —SO₃H, more particularly —SR⁴ wherein R⁴ is hydrogen, alkyl, or —O₃H or —SO₃H. In embodiments of the invention, an epi-cyclohexanehexyl compound, salt, or derivative thereof, in particular a pure or substantially pure epi-cyclohexanehexyl compound, is used in the formulations, dosage forms, methods and uses disclosed herein.

In aspects of the invention, the cyclohexane polyalcohol compound is an epi-cyclohexanehexyl compound, in particular pure or substantially pure epi-cyclohexanehexyl compound.

An “epi-cyclohexanehexyl compound” includes compounds having the base structure of formula VI:

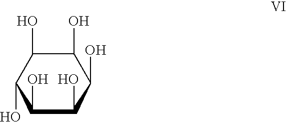

An epi-cyclohexanehexyl compound includes a compound of the formula VI wherein one, two, three or four, preferably one, two or three, more preferably one or two hydroxyl groups are replaced by substituents, in particular univalent substituents, with retention of configuration. Suitable substituents include without limitation hydrogen, alkyl, acyl, alkenyl, cycloalkyl, halogen, —NHR¹ wherein R¹ is hydrogen, acyl, alkyl or —R²R³ wherein R² and R³ are the same or different and represent acyl or alkyl; —PO₃H₂; —SR⁴ wherein R⁴ is hydrogen, alkyl, or —O₃H; and —OR³ wherein R³ is hydrogen, alkyl, or —SO₃H.

Particular aspects of the invention utilize epi-cylcohexanehexyl compounds of the formula VI wherein one or more of the hydroxyl groups is replaced with alkyl, acyl, alkenyl, —NHR¹ wherein R¹ is hydrogen, acyl, alkyl or —R²R³ wherein R² and R³ are the same or different and represent acyl or alkyl; —SR⁴ wherein R⁴ is hydrogen, alkyl, or —O₃H; and —OR³ wherein R³ is hydrogen, alkyl, or —SO₃H, more particularly —SR⁴ wherein R⁴ is hydrogen, alkyl, or —O₃H or —SO₃H.

In aspects of the invention, the cyclohexane polyalcohol compound is epi-cyclohexanehexyl (i.e., epi-inositol), in particular pure or substantially pure epi-inositol.

In embodiments of the invention, the cyclohexane polyalcohol compound is an isolated, in particular pure, more particularly, substantially pure, compound of the formula II wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In particular aspects of the invention, a cyclohexane polyalcohol compound does not include a compound of the formula I or II where (a) when one of R¹, R², R³, R⁴, R⁵, and/or R⁶ are alkyl or fluorine, more than 4 of the other of R¹, R², R³, R⁴, R⁵, and/or R⁶ are hydroxyl, (b) when one of R¹, R², R³, R⁴, R⁵, and/or R⁶ is amino or azide, more than four of R¹, R², R³, R⁴, R⁵, and/or R⁶ are hydroxyl, (c) when two of R¹, R², R³, R⁴, R⁵, and/or R⁶ are amino, more than three of R¹, R², R³, R⁴, R⁵, and/or R⁶ are hydroxyl, and (d) R¹, R², R³, R⁴, R⁵, and/or R⁶ are isopropylidene.

In some aspects of the invention, a cyclohexane polyalcohol compound is utilized where one or more of R¹, R², R³, R⁴, R⁵, and/or R⁶ are alkyl, alkoxy, or halo, and the other of R¹, R², R³, R⁴, R⁵, and/or R⁶ is hydrogen.

In embodiments of the invention, the cyclohexane polyalcohol compound is a compound of the formula I or II where the hydrogen at one or more of positions 1, 2, 3, 4, 5, or 6 of formula I or II is substituted with a radical disclosed herein for R¹, R², R³, R⁴, R⁵, and R⁶, including optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular optionally substituted alkyl, alkenyl, alkoxy, amino, imino, thiol, nitro, cyano, halo, or carboxyl.

In embodiments of the invention, the cyclohexane polyalcohol compound is a compound of the formula I or II wherein one or more of, two or more of, or three or more of R¹, R², R³, R⁴, R⁵, and/or R⁶ are independently alkenyl, alkynyl, alkylene, alkenylene, alkoxy, alkenyloxy, cycloalkenyl, cycloalkoxy, aryl, aryloxy, arylalkoxy, aroyl, heteroaryl, heterocyclic, acyl, acyloxy, sulfonyl, sulfenyl, sulfinyl, sulfonate, sulfoxide, sulfate, nitro, cyano, isocyanato, thioaryl, thioalkoxy, seleno, silyl, silyloxy, silylthio, Cl, I, Br, carboxyl, carboxylic ester, carbonyl, carbamoyl, or carboxamide and the other of R¹, R², R³, R⁴, R⁵, and/or R⁶ is a hydroxyl.

In embodiments of the invention, the cyclohexane polyalcohol compound is an isolated, in particular pure, more particularly, substantially pure, compound of the formula I or II wherein one or more of, two or more of, or three or more of R¹, R², R³, R⁴, R⁵, and/or R⁶ are independently C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, C₃-C₈ cycloalkoxy, acyloxy, sulfonyl, sulfenyl, sulfinyl, sulfonate, sulfoxide, sulfate, isocyanato, thioaryl, thioalkoxy, selene, silyl, silyloxy, silythio, aryl, aroyl, aryloxy, arylC₁-C₆alkoxy, acetyl, heteroaryl, heterocyclic, amino, thiol, thioalkyl, thioalkoxy, nitro, cyano, halo (e.g., Cl, I, or Br), carboxyl, carboxylic ester, carbonyl, carbamoyl, or carboxamide and the other of R¹, R², R³, R⁴, R⁵, and/or R⁶ is a hydroxyl. In particular aspects, (a) when one of R¹, R², R³, R⁴, R⁵, and/or R⁶ are alkyl or fluorine no more than 4 of the other of R¹, R², R⁵, and/or R⁶ are hydroxyl, (b) when one of R¹, R², R³, R⁴, R⁵, and/or R⁶ is amino no more than four of R¹, R², R³, R⁴, R⁵, and/or R⁶ are hydroxyl, (c) when two of R¹, R², R³, R⁴, R⁵, and/or R⁶ are amino, no more than three of R¹, R², R³, R⁴, R⁵, and R⁶ are hydroxyl, and (d) R¹, R², R³, R⁴, R⁵, and/or R⁶ are not isopropylidene.

In embodiments of the invention, the cyclohexane polyalcohol compound is a compound of the formula I wherein R² is hydroxyl in an equatorial position, at least one, two, three, or four of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfenyl, sulfonyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexane polyalcohol compound is a compound of the formula I wherein R² is hydroxyl in an equatorial position, at least two of R¹, R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br, and the other of R¹, R³, R⁴, R⁵, and/or R⁶ are hydroxyl.

In embodiments of the invention, the cyclohexane polyalcohol compound is a compound of the formula I or II wherein at least two of R¹, R², R³, R⁴, R⁵, and/or R⁶ are hydroxyl, and one, two, three or four or more of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein at least two of R¹, R², R³, R⁴, R⁵, and/or R⁶ are hydroxyl, and two or more of the other of R¹, R², R³, R⁴, R⁵, and/or R⁶ are alkyl, cycloalkyl, alkenyl, cycloalkenyl, alkynyl, alkylene, alkenylene, alkoxy, alkenyloxy, cycloalkoxy, aryl, aryloxy, arylalkoxy, aroyl, heteroaryl, heterocyclic, acyl, or acyloxy, sulfonyl, sulfenyl, sulfinyl, amino, imino, cyano, isocyanato, seleno, silyl, silyloxy, silylthio, thiol, thioalkyl, thioalkoxy, halo, carboxyl, carboxylic ester, carbonyl, carbamoyl, and carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein at least two of R¹, R², R³, R⁴, R⁵, and/or R⁶ are hydroxyl, and three or more of the other of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein at least three of R¹, R², R³, R⁴, R⁵, and/or R⁶ are hydroxyl, and one, two, or three of the other of R¹, R², R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein at least four of R¹, R², R³, R⁴, R⁵, and/or R⁶ are hydroxyl, and one or two of the other of R¹, R³, R⁴, R⁵, and/or R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfonate, sulfenyl, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein R¹, R², R⁴, R⁵, and R⁶ are hydroxyl, and R³ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R³ is selected from the group consisting of alkenyl, alkynyl, alkylene, alkenylene, alkoxy, alkenyloxy, cycloalkyl, cycloalkenyl, cycloalkoxy, aryl, aryloxy, arylalkoxy, aroyl, imino, heteroaryl, heterocyclic, acyl, acyloxy, sulfonyl, sulfenyl, sulfinyl, sulfoxide, sulfate, thioalkoxy, thioaryl, carboxyl, carbonyl, carbamoyl, or carboxamide, in particular alkoxy, sulfonyl, sulfenyl, sulfinyl, sulfoxide, sulfate, thioalkoxy, carboxyl, carbonyl, carbamoyl, or carboxamide. In a particular embodiment, R³ is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester, in particular C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, arylC₁-C₆alkoxy, Cl, I, or Br.

In embodiments of the invention, the cyclohexane polyalcohol compound is a compound of the formula I or II wherein R¹, R³, R⁴, R⁵, and R⁶ are hydroxyl, and R² is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carboxylic ester, carbonyl, carbamoyl, or carboxamide. In embodiments, R² is selected from the group consisting of C₁-C₆ alkyl, C₃-C₆ alkenyl, C₂-C₆ alkynyl, C₂-C₆ alkylene, C₂-C₈ alkenylene, C₁-C₆ alkoxy, C₂-C₆ alkenyloxy, C₃-C₈ cycloalkyl, C₃-C₈ cycloalkenyl, C₃-C₈ cycloalkoxy, aryl, aryloxy, arylC₁-C₆alkoxy, acetyl, halo, and carboxylic ester.

In embodiments of the invention, the cyclohexane polyalcohol compound is a compound of the formula I, II, III or IV wherein one, two, three, four or five of R¹, R², R³, R⁴, R⁵, and/or R⁶ are each independently:
(a) alkyl with 1 to 24 carbon atoms, in particular 1 to 10 or 1 to 6 carbon atoms;
(b) cycloalkyl with 3 to 16 carbon atoms, in particular 3 to 10 or 3 to 6 carbon atoms;
(c) alkenyl with 2 to 24 carbon atoms, in particular 2 to 10 or 2 to 6 carbon atoms;
(d) cycloalkenyl with 4 to 16 carbon atoms, in particular 4 to 10 or 4 to 6 carbon atoms;
(e) aryl with 4 to 24 carbon atoms, in particular 4 to 10, 4 to 8, or 6 or carbon atoms;
(f) aralkyl, alkaryl, aralkenyl, or alkenylaryl;
(g) heterocyclic group comprising 3 to 10, in particular 3 to 8 or 3 to 6 ring members and at least one atom selected from the group consisting of oxygen, nitrogen, and sulfur;
(h) alkoxy with 1 to 6 carbon atoms or 1 to 3 carbon atoms in particular methoxy, ethoxy, propoxy, butoxy, isopropoxy or tert-butoxy, especially methoxy, or
(i) halo, in particular fluorine, chlorine, or bromine, especially chlorine.

In embodiments of the invention, the cyclohexane polyalcohol compound is a compound of the formula I, II, III or IV wherein R² is hydroxyl and one, two, three, four or five of R¹, R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cyclopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexane polyalcohol compound is a compound of the formula I, II, III or IV wherein R¹ is hydroxyl and one, two, three, four or five of R², R³, R⁴, R⁵, and/or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, methoxy, ethoxy, propoxy, butoxy, isopropoxy, tert-butoxy, chloro, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cyclopentadienyl, cyclohexenyl, cyclohexadienyl, phenyl, biphenyl, terphenyl, naphtyl, anthracenyl, phenanthrenyl, pyridyl, furyl, or thiazolyl.

In embodiments of the invention, the cyclohexane polyalcohol compound is a compound of the formula I, II, III or IV wherein one or two of R¹, R², R³, R⁴, R⁵, and/or R⁶ are carboxyl, carbamyl, sulfonyl, or a heterocyclic comprising a N atom, more particularly N-methylcarbamyl, N-propylcarbamyl, N-cyanocarbamyl, aminosulfonyl, isoxazolyl, imidazolyl, and thiazolyl.

In embodiments of the invention, the cyclohexane polyalcohol compound is a compound of the formula I, II, III or IV where R² is hydroxyl; and R¹, R³, R⁴, R⁵, and R⁶ are independently selected from C₁-C₆alkyl, C₂-C₆alkenyl, C₂-C₆alkynyl, C₁C₆ alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀ heterocyclic, C₁-C₆acyl, C₁-C₆acyloxy, hydroxyl, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀ cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic; provided that R¹, R², R³, R⁴, R⁵, and R⁶ are not all hydroxyl.

In embodiments of the invention, the cyclohexane polyalcohol compound is a compound of the formula I, II, III or IV where R² is hydroxyl; one of R¹, R³, R⁴, R⁵, and R⁶ is hydroxyl; and four of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexane polyalcohol compound is a compound of the formula I, II, III or IV where R² is hydroxyl; two of R¹, R³, R⁴, R⁵, and R⁶ are hydroxyl; and three of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexane polyalcohol compound is a compound of the formula III or IV where R² is hydroxyl; three of R¹, R³, R⁴, R⁵, and R⁶ is hydroxyl; and two of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexane polyalcohol compound is a compound of the formula III or IV where R² is hydroxyl; four of R¹, R³, R⁴, R⁵, and R⁶ are hydroxyl; and one of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀ aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexane polyalcohol compound is a compound of the formula III or IV wherein one of R¹, R³, R⁴, R⁵, and R⁶ is C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than four of the remainder of R¹, R², R³, R⁴, R⁵, and R⁶ are hydroxyl.

In embodiments of the invention, the cyclohexane polyalcohol compound is a compound of the formula III or IV wherein two of R¹, R³, R⁴, R⁵, and R⁶ are C₁-C₆alkyl, C₁-C₆alkoxy, C₁-C₆acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than three of R¹, R², R³, R⁴, R⁵, and R⁶ are hydroxyl.

In embodiments of the invention, the cyclohexane polyalcohol compound is a compound of the formula III or IV wherein three of R¹, R³, R⁴, R⁵, and R⁶ are C₁-C₆alky, C₁-C₆alkoxy, C₁-C₆alkyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than two of R¹, R², R³, R⁴, R⁵, and R⁶ are hydroxyl.

In embodiments of the invention, the cyclohexane polyalcohol compound is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexane polyalcohol compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexane polyalcohol compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexane polyalcohol compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, alkoxy, acetyl, halo, carboxylic ester, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexane polyalcohol compound is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, the cyclohexane polyalcohol compound is a compound of the formula I, II, III or IV wherein one, two, or three of R¹, R², R³, R⁴, R⁵, and/or R⁶ is each independently —OR¹⁵ where R¹⁵ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide or a carbohydrate. In an aspect, wherein one, two, or three of R¹, R², R³, R⁴, R⁵, and/or R⁶ is each independently —OR¹⁵ where R¹⁵ is C₁-C₆ alkyl, most particularly C₁-C₃ alkyl.

In selected cyclohexane polyalcohol compounds of the formula I, II, III or IV, at least one of R¹, R², R³, R⁴, R⁵, and/or R⁶ is —OR²⁰ wherein R²⁰ is —CF₃, CF₃CF₂, CF₃CH₂, CH₂NO₂, CH₂NH₂, C(CH₂)₃, or cyclopropyl.

In embodiments of the invention, the cyclohexane polyalcohol compound is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁵ are hydroxyl and R⁶ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁵ are hydroxyl and R⁶ is methoxy.

In embodiments of the invention, the cyclohexane polyalcohol compound is a compound of the formula I, II, III or IV wherein R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁶ are hydroxyl and R⁵ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁴, and R⁶ are hydroxyl and R⁵ is methoxy.

In embodiments of the invention, the cyclohexane polyalcohol compound is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R³, R⁵, and R⁶ are hydroxyl and R⁴ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R³, R⁵, and R⁶ are hydroxyl and R⁴ is methoxy.

In embodiments of the invention, the cyclohexane polyalcohol compound is a compound of the formula I, II, III or IV wherein R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R⁴, R⁵, and R⁶ are hydroxyl and R³ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R², R⁴, R⁵, and R⁶ are hydroxyl and R³ is methoxy.

In embodiments of the invention, the cyclohexane polyalcohol compound is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R³, R⁴, R⁵, and R⁶ are hydroxyl and R² is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R¹, R³, R⁴, R⁵, and R⁶ are hydroxyl and R² is methoxy.

In embodiments of the invention, the cyclohexane polyalcohol compound is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, which may be substituted with alkyl, halo (e.g., fluoro), substituted alkyl (e.g. alkylhalo, haloalkylhalo, alkylhaloalkyl), cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R², R³, R⁴, R⁵, and R⁶ are hydroxyl and R¹ is —OR²⁰ wherein R²⁰ is CF₃, CF₃CF₂, CF₃CH₂, CH₂NO₂, CH₂NH₂, C(CH₂)₃, or cyclopropyl. In another particular embodiment of the invention, R², R³, R⁴, R⁵, and R⁶ are hydroxyl and R¹ is methoxy.

In embodiments of the invention, the cyclohexane polyalcohol compound is a compound of the formula III or IV, wherein two, three, four or five of R¹, R², R³, R⁴, R⁵, or R⁶ are hydroxyl; at least one of R¹, R², R³, R⁴, R⁵, or R⁶ is optionally substituted alkoxy; and the remainder of R¹, R², R³, R⁴, R⁵, or R⁶ if any are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆acyl, C₁-C₆ acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀arylC₁-C₃alkyl, C₆-C₁₀heteroaryl and C₃-C₁₀heterocyclic.

In embodiments of the invention, the cyclohexane polyalcohol compound is a compound of the formula III or IV, wherein five of R¹, R², R³, R⁴, R⁵, or R⁶ are hydroxyl; and one of R¹, R², R³, R⁴, R⁵, or R⁶ is C₁-C₆alkoxy; for example at least one of R¹, R², R³, R⁴, R⁵, or R⁶ is methoxy.

In embodiments of the invention, the cyclohexane polyalcohol compound is a compound of the formula IV, wherein two, three, or four of R², R³, R⁴, R⁵, or R⁶ are hydroxyl; R¹ is optionally substituted alkoxy; and the remainder of R², R³, R⁴, R⁵, or R⁶ are independently selected from C₁-C₆alkyl, C₂-C₆alkenyl, C₂-C₆alkynyl, C₁-C₆alkoxy, C₂-C₆alkenyloxy, C₃-C₁₀cycloalkyl, C₁-C₆acyl, C₁-C₆acyloxy, hydroxyl, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexane polyalcohol compound is a compound of the formula IV, wherein R¹ is C₁-C₆ alkoxy; and R², R³, R⁴, R⁵, and R⁶ are hydroxyl; for example R¹ is methoxy.

In embodiments of the invention, the cyclohexane polyalcohol compound is a compound of the formula I, II, III or IV wherein five of R¹, R², R³, R⁴, R⁵, and/or R⁶ are hydroxyl and the other of R¹, R², R³, R⁴, R⁵, and/or R⁶ is substituted alkoxy, in particular alkoxy having about 1-6 carbon atoms, more particularly methoxy, ethoxy, propoxy, butoxy, isopropoxy and tert-butoxy, substituted with alkyl, in particular C₁-C₆ alkyl, more particularly C₁-C₃ alkyl.

In embodiments of the invention, the cyclohexane polyalcohol compound is a compound of the formula I, II, III or IV wherein five of R¹, R², R³, R⁴, R⁵, and/or R⁶ are hydroxyl and the other of R¹, R², R³, R⁴, R⁵, and/or R⁶ is alkoxy, in particular alkoxy having about 1-6 carbon atoms, more particularly methoxy, ethoxy, propoxy, butoxy, isopropoxy and tert-butoxy substituted with halo (e.g., fluoro, chloro or bromo) which may be substituted. In particular embodiments five of R¹, R², R³, R⁴, R⁵, and/or R⁶ are hydroxyl and the other of R¹, R², R³, R⁴, R⁵, and/or R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein five of R¹, R², R³, R⁴, R⁵, and/or R⁶ are hydroxyl and the other of R¹, R², R³, R⁴, R⁵, and/or R⁶ is a haloalkoxyalkyl, in particular fluoromethoxymethyl, chloromethoxyethyl, trifluoromethoxymethyl, difluoromethoxyethyl, or trifluoroethoxymethyl.

In embodiments of the invention, the cyclohexane polyalcohol compound is a compound of the formula I, II, III or IV wherein R¹, R², R³, R⁴, and R⁵ are hydroxyl and R⁶ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, the cyclohexane polyalcohol compound is a compound of the formula I, II, III or IV wherein R¹, R², R³, R⁴, and R⁶ are hydroxyl and R⁵ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexane polyalcohol compound is a compound of the formula I, II, III or IV wherein R¹, R², R³, R⁵, and R⁶ are hydroxyl and R⁴ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexane polyalcohol compound is a compound of the formula I, II, III or IV wherein R¹, R², R⁴, R⁵, and R⁶ are hydroxyl and R³ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexane polyalcohol compound is a compound of the formula I, II, III or IV wherein R¹, R³, R⁴, R⁵, and R⁶ are hydroxyl and R² is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexane polyalcohol compound is a compound of the formula I, II, III or IV wherein R², R³, R⁴, R⁵, and R⁶ are hydroxyl and R¹ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl, more particularly C₁-C₃ alkyl.

In embodiments of the invention, the cyclohexane polyalcohol compound is a compound of the formula I, II, III or IV wherein R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁵ are hydroxyl and R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R⁴, R⁵, and R⁶ are hydroxyl and R³ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R³, R⁴, R⁵, and R⁶ are hydroxyl and R² is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is a compound of the formula I, II, III or IV wherein R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R², R³, R⁴, R⁵, and R⁶ are hydroxyl and R¹ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, the cyclohexane polyalcohol compound is methyl-scyllo-inositol

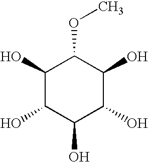

In embodiments of the invention, the cyclohexane polyalcohol compound is a compound of the formula I, II, III or IV wherein one, two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester. In aspects of the invention at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexane polyalcohol compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexane polyalcohol compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is a carboxylic ester.

In embodiments of the invention, the cyclohexane polyalcohol compound is a compound of the formula I, II, III or IV wherein five of R¹, R², R³, R⁴, R⁵, or R⁶ are hydroxyl and the other of R¹, R², R³, R⁴, R⁵, or R⁶ is a carboxylic ester.

In embodiments of the invention, the cyclohexane polyalcohol compound is a compound of the formula I, II, III or IV wherein at least one of R¹, R², R³, R⁴, R⁵, and/or R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R¹, R², R³, R⁴, and R⁵ are hydroxyl and R⁶ is a carboxylic ester. In aspects of the invention, R⁶ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R¹, R², R³, R⁴, and R⁶ are hydroxyl and R⁵ is a carboxylic ester. In aspects of the invention, R⁵ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R¹, R², R³, R⁵, and R⁶ are hydroxyl and R⁴ is a carboxylic ester. In aspects of the invention, R⁴ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R¹, R², R⁴, R⁵, and R⁶ are hydroxyl and R³ is a carboxylic ester. In aspects of the invention, R³ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R¹, R³, R⁴, R⁵, and R⁶ are hydroxyl and R² is a carboxylic ester. In aspects of the invention, R² is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, the cyclohexane polyalcohol compound is a compound of the formula I, II, III or IV wherein R², R³, R⁴, R⁵, and R⁶ are hydroxyl and R¹ is a carboxylic ester. In aspects of the invention, R¹ is —C(O)OR¹⁴ where R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic. In particular embodiments, R¹⁴ is selected to provide an amino acid derivative or an ester derivative. In preferred embodiments of the invention R¹⁴ is one of the following:

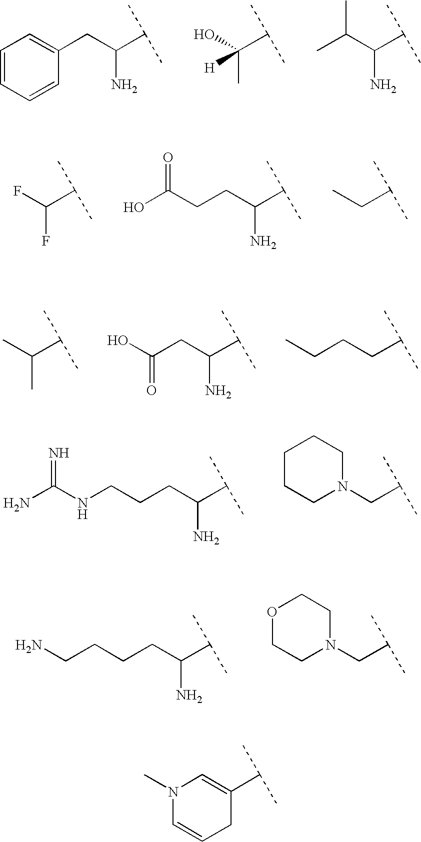

In embodiments of the invention, the cyclohexane polyalcohol compound is a compound of the formula I, II, III or IV wherein one, two or three of R¹, R², R³, R⁴, R⁵, and/or R⁶ is each independently:

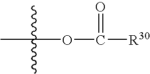

where R³⁰ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, and the other of R¹, R², R³, R⁴, R⁵, and/or R⁶ is hydroxyl.

In embodiments of the invention, the cyclohexane polyalcohol compound is a compound of the formula I, II, III or IV wherein at least one, two, three or four of R¹, R³, R⁴, R⁵, and/or R⁶ are hydroxyl and the other of R¹, R³, R⁴, R⁵, and/or R⁶ are alkyl, halo, alkoxy, sulfonyl, sulfinyl, thiol, thioalkyl, thioalkoxy, carboxyl, in particular C₁-C₆ alkyl, C₁-C₆ alkoxy, or halo.

In embodiments of the invention, the cyclohexane polyalcohol compound is a compound of the formula I, II, III or IV wherein R¹, R², R³, R⁴, R⁵, and/or R⁶ is each independently —CH₃, —OCH₃, F, N₃, NH₂, SH, NO₂, CF₃, OCF₃, SeH, Cl, Br, I or CN with the proviso that four or five of R¹, R², R³, R⁴, R⁵, and/or R⁶ are hydroxyl.

In embodiments of the invention, the cyclohexane polyalcohol compound is a compound of the formula I, II, III or IV wherein five of R¹, R², R³, R⁴, R⁵, and/or R⁶ are hydroxyl and one of R¹, R², R³, R⁴, R⁵, or R⁶, and more particularly R² or R³, is selected from the group consisting of —CH₃, —OCH₃, CF₃, F, SeH, Cl, Br, I and CN.

In embodiments of the invention, the cyclohexane polyalcohol compound is a compound of the formula I, II, III or IV wherein four of R¹, R², R³, R⁴, R⁵, and/or R⁶ are hydroxyl and two of R¹, R², R³, R⁴, R⁵, and/or R⁶ are selected from the group consisting of —CH₃, —OCH₃, CF₃, F, —NO₂, SH, SeH, Cl, Br, I and CN.

In embodiments of the invention, the cyclohexane polyalcohol compound is a compound of the formula III or IV, wherein four of R¹, R², R³, R⁴, R⁵, or R⁶ are hydroxyl; and one of R¹, R², R³, R⁴, R⁵, or R⁶ is each independently selected from the group CH₃, OCH₃, NO₂, CF₃, OCF₃, F, Cl, Br, I and CN.

In embodiments of the invention, the cyclohexane polyalcohol compound is a compound of the formula III or IC, wherein four of R¹, R², R³, R⁴, R⁵, formula III or IV, wherein five of R¹, R², or R⁶ are hydroxyl; and one of R¹, or R⁶ is selected from CH₃, OCH₃, NO₂, CF₃, OCF₃, F, Cl, Br, I and CN.

In embodiments of the invention, the cyclohexane polyalcohol compound is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower alkyl, especially methyl, ethyl, butyl, or propyl, preferably methyl.

In embodiments of the invention, the cyclohexane polyalcohol compound is a compound of the formula I, II, III or IV wherein four of R¹, R², R³, R⁴, R⁵, and/or R⁶ are hydroxyl and the other two of R¹, R², R³, R⁴, R⁵, and/or R⁶ are lower cycloalkyl, especially cyclopropyl, cyclobutyl, and cyclopentyl.

In embodiments of the invention, the cyclohexane polyalcohol compound is a compound of the formula I, II, III or IV wherein two, three, four or five of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexane polyalcohol compound is a compound of the formula I, II, III or IV wherein two of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexane polyalcohol compound is a compound of the formula I, II, III or IV wherein three of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, and/or R⁶ is halo, in particular fluoro, chloro or bromo, more particularly chloro.

In embodiments of the invention, the cyclohexane polyalcohol compound is a compound of the formula I, II, III or IV wherein four of R¹, R², R³, R⁴, R⁵, and/or R⁶ are hydroxyl, the other of R¹, R², R³, R⁴, R⁵, and/or R⁶ are independently hydrogen, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide, especially alkyl, amino, imino, azido, thiol, thioalkyl, nitro, thioalkoxy, cyano, or halo, preferably C₁-C₆ alkyl, C₁-C₆ alkoxy, acetyl, halo, or carboxylic ester, and at least one of R¹, R², R³, R⁴, R⁵, or R⁶ is halo, in particular fluoro, chloro or bromo, more particularly chloro.

In embodiments of the invention, the cyclohexane polyalcohol compound is a compound of the formula III or IV, wherein two, three, four or five of R¹, R², R³, R⁴, R⁵, or R⁶ are hydroxyl; at least one of R¹, R², R³, R⁴, R⁵, or R⁶ is halo; and the remainder of R¹, R², R³, R⁴, R⁵, or R⁶, if any, are independently C₁-C₆alkyl, C₂-C₆ alkenyl, C₂-C₆alkynyl, C₁C₆alkoxy, C₂-C₆alkenyloxy, C₃-C₁₀cycloalkyl, C₁-C₆acyl, C₁-C₆ acyloxy, —NH₂, —NHR⁷, —NR⁷R⁸—, ═NR⁷, —S(O)₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C_{10 B}heterocyclic.

In still another aspect, the cyclohexane polyalcohol compound is a compound of formula III or IV, wherein four of R¹, R², R³, R⁴, R⁵, or R⁶ are hydroxyl; one of R¹, R², R³, R⁴, R⁵, or R⁶ is halo; and one of R¹, R², R³, R⁴, R⁵, or R⁶ is selected from C₁-C₆alkyl, C₂-C₆alkenyl, C₂-C₆alkynyl, C₁C₆alkoxy, C₂-C₆alkenyloxy, C₃-C₁₀cycloalkyl, C₁-C₆ acyl, C₁-C₆ acyloxy, hydroxyl, —NH₂, —NHR⁷, —NR⁷R⁸—, —NR⁷, —S(O)₂R⁷, —SH, nitro, cyano, halo, haloalkyl, haloalkoxy, hydroxyalkyl, —Si(R^{7P})B_{3B}, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀heterocyclic, and at least one of R¹, R², R³, R⁴, R⁵, or R⁶ is halo.

In embodiments of the invention, the cyclohexane polyalcohol compound is a compound of the formula I, II, III or IV wherein five of R¹, R², R³, R⁴, R⁵, and/or R⁶ are hydroxyl and the other of R¹, R², R³, R⁴, R⁵, and/or R⁶ is halo, in particular fluoro, chloro or bromo, more particularly chloro.

In embodiments of the invention, the cyclohexane polyalcohol compound is a compound of the formula I, II, III or IV wherein R¹, R², R³, R⁴, and R⁵ are hydroxyl and R⁶ is halo, in particular fluorine, chlorine or bromine, more particularly chloro. In a particular embodiment of the invention, R¹, R², R³, R⁴, and R⁵ are hydroxyl and R⁶ is chloro.

In embodiments of the invention, the cyclohexane polyalcohol compound is a compound of the formula I, II, III or IV wherein R¹, R², R³, R⁴, and R⁶ are hydroxyl and R⁵ is halo, in particular fluoro, chloro or bromo, more particularly chloro. In a particular embodiment of the invention, R¹, R², R³, R⁴, and R⁶ are hydroxyl and R⁵ is chloro.

In embodiments of the invention, the cyclohexane polyalcohol compound is a compound of the formula I, II, III or IV wherein R¹, R², R³, R⁵, and R⁶ are hydroxyl and R⁴ is halo, in particular fluoro, chloro or bromo, more particularly chloro. In a particular embodiment of the invention, R¹, R², R³, R⁵, and R⁶ are hydroxyl and R⁴ is chloro.

In embodiments of the invention, the cyclohexane polyalcohol compound is a compound of the formula I, II, III or IV wherein R¹, R², R⁴, R⁵, and R⁶ are hydroxyl and R³ is halo, in particular fluoro, chloro or bromo, more particularly chloro. In a particular embodiment of the invention, R¹, R², R⁴, R⁵, and R⁶ are hydroxyl and R³ is chloro.

In embodiments of the invention, the cyclohexane polyalcohol compound is a compound of the formula I, II, III or IV wherein R¹, R³, R⁴, R⁵, and R⁶ are hydroxyl and R² is halo, in particular fluoro, chloro or bromo, more particularly chloro. In a particular embodiment of the invention, R¹, R³, R⁴, R⁵, and R⁶ are hydroxyl and R² is chloro.

In embodiments of the invention, the cyclohexane polyalcohol compound is a compound of the formula I, II, III or IV wherein R², R³, R⁴, R⁵, and R⁶ are hydroxyl and R¹ is halo, in particular fluoro, chloro or bromo, more particularly chloro. In a particular embodiment of the invention, R², R³, R⁴, R⁵, and R⁶ are hydroxyl and R¹ is chloro.

In embodiments of the invention, the cyclohexane polyalcohol compound is 1-chloro-1-deoxy-scyllo-inositol.

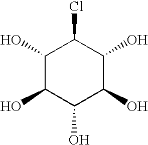

Cyclohexane polyalcohol compounds utilized in the invention may be prepared using reactions and methods generally known to the person of ordinary skill in the art, having regard to that knowledge and the disclosure of this application. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing cyclohexane polyalcohol compounds are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and cyclohexane polyalcohol compounds may be isolated and purified using conventional techniques, such as precipitation, filtration, distillation, crystallization, chromatography, and the like. The compounds may be characterized using conventional methods, including physical constants and spectroscopic methods, in particular HPLC.

Cyclohexane polyalcohol compounds which are basic in nature can form a wide variety of different salts with various inorganic and organic acids. In practice it is desirable to first isolate a cyclohexane polyalcohol compound from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Cyclohexane polyalcohol compounds which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Scyllo-cyclohexane polyalcohol compounds can be prepared using conventional processes or they may be obtained from commercial sources. For example, scyllo-cyclohexane polyalcohol compounds can be prepared using chemical and/or microbial processes. In aspects of the invention, a scyllo-inositol is produced using process steps described by M. Sarmah and Shashidhar, M., Carbohydrate Research, 2003, 338, 999-100, Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med. Chem. 1999, 7(11):2577-89; or Kiely D. E., and Fletcher, H. G., J. American Chemical Society (US) 1968, 90:3289-3290; described in JP09-140388, DE 3,405,663 (Merck patent GMBH), JP04-126075, JP05-192163, or WO06109479, or described in WO0503577, US20060240534, EP1674578, JP9140388, JP09140388, JP02-184912, JP03-102492 (Hokko Chemical Industries). In particular aspects of the compositions and methods of the invention, a scyllo-inositol is prepared using the chemical process steps described in Husson, C., et al, Carbohydrate Research 307 (1998) 163-165. In other aspects of the compositions and methods of the invention, a scyllo-inositol is prepared using microbial process steps similar to those described in WO05035774 (EP1674578 and US20060240534) JP2003102492, or JP09140388 (Hokko Chemical Industries). Derivatives may be produced by introducing into a scyllo-cyclohexanehexyl using methods well known to a person of ordinary skill in the art.

In aspects of the invention, an epi-inositol can be prepared using chemical and/or microbial processes. For example, an epi-inositol may be prepared by the process described by V. Pistarà (Tetrahedron Letters 41, 3253, 2000), Magasanik B., and Chargaff E. (J Biol Chem, 1948, 174:173188), U.S. Pat. No. 7,157,268, or in PCT Published Application No. WO0075355 Derivatives may be produced by introducing substituents into an epi-inositol using methods well known to a person of ordinary skill in the art.

A cyclohexane polyalcohol compound may additionally comprise a carrier, including without limitation one or more of a polymer, carbohydrate, peptide or derivative thereof. A carrier may be substituted with substituents described herein including without limitation one or more alkyl, amino, nitro, halogen, thiol, thioalkyl, sulfate, sulfonyl, sulfenyl, sulfinyl, sulfoxide, hydroxyl groups. A carrier can be directly or indirectly covalently attached to a compound of the invention. In aspects of the invention the carrier is an amino acid including alanine, glycine, proline, methionine, serine, threonine, or asparagine. In other aspects the carrier is a peptide including alanyl-alanyl, prolyl-methionyl, or glycyl-glycyl.

A carrier also includes a molecule that targets a compound of the invention to a particular tissue or organ. In particular, a carrier may facilitate or enhance transport of a compound of the invention to the brain by either active or passive transport.

A “polymer” as used herein refers to molecules comprising two or more monomer subunits that may be identical repeating subunits or different repeating subunits. A monomer generally comprises a simple structure, low-molecular weight molecule containing carbon. Polymers can be optionally substituted. Examples of polymers which can be used in the present invention are vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In particular aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The simplest carbohydrates are monosaccharides, which are small straight-chain aldehydes and ketones with many hydroxyl groups added, usually one on each carbon except the functional group. Examples of monosaccharides include erythrose, arabinose, allose, altrose, glucose, mannose, threose, xylose, gulose, idose, galactose, talose, aldohexose, fructose, ketohexose, ribose, and aldopentose. Other carbohydrates are composed of monosaccharide units, including disaccharides, oligosaccharides, or polysaccharides, depending on the number of monosaccharide units. Disaccharides are composed of two monosaccharide units joined by a covalent glycosidic bond. Examples of disaccharides are sucrose, lactose, and maltose. Oligosaccharides and polysaccharides, are composed of longer chains of monosaccharide units bound together by glycosidic bonds. Oligosaccharides generally contain between 3 and 9 monosaccharide units and polysaccharides contain greater than 10 monosaccharide units. A carbohydrate group may be substituted at one two, three or four positions, other than the position of linkage to a compound of the formula I, II, III or IV. For example, a carbohydrate may be substituted with one or more alkyl, amino, nitro, halo, thiol, carboxyl, or hydroxyl groups, which are optionally substituted. Illustrative substituted carbohydrates are glucosamine or galactosamine.

In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is selected from the group consisting of glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Suitable pentose sugars include arabinose, fucose, and ribose.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” for use as a carrier in the practice of the present invention includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. In aspects of the invention the carrier is an amino acid such as alanine, glycine, proline, methionine, serine, threonine, histidine, or asparagine. In other aspects the carrier is a peptide such as alanyl-alanyl, prolyl-methionyl, or glycyl-glycyl. In still other aspects, the carrier is a polypeptide such as albumin, antitrypsin, macroglobulin, haptoglobin, caeruloplasm, transferrin, α- or β-lipoprotein, β- or γ-globulin or fibrinogen.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition. 3:55; Morgan, B. A. and Gainor, J. A. (1989) Aim. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptide analogues, derivatives and peptidomimetics include peptides substituted with one or more benzodiazepine molecules (see e.g., James, G. L. et al. (1993) Science 260:1937-1942), peptides with methylated amide linkages and “retro-inverso” peptides (see U.S. Pat. No. 4,522,752 by Sisto).

Examples of peptide derivatives include peptides in which an amino acid side chain, the peptide backbone, or the amino- or carboxy-terminus has been derivatized (e.g., peptidic compounds with methylated amide linkages).

The term mimetic, and in particular, peptidomimetic, is intended to include isosteres. The term “isostere” refers to a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Other examples of isosteres include peptides substituted with one or more benzodiazepine molecules (see e.g., James, G. L. et al. (1993) Science 260:1937-1942)

Other possible modifications include an N-alkyl (or aryl) substitution ([CONR]), backbone crosslinking to construct lactams and other cyclic structures, substitution of all D-amino acids for all L-amino acids within the compound (“inverso” compounds) or retro-inverso amino acid incorporation ([NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A peptide can be attached to a compound of the invention through a functional group on the side chain of certain amino acids (e.g. serine) or other suitable functional groups. In embodiments of the invention the carrier may comprise four or more amino acids with groups attached to three or more of the amino acids through functional groups on side chains. In another embodiment, the carrier is one amino acid, in particular a sulfonate derivative of an amino acid, for example cysteic acid.

“Disorders and/or diseases”, “disorder(s)” and “disease(s)” are used interchangeably herein and include a condition characterized by abnormal protein folding or aggregation or abnormal amyloid formation, deposition, accumulation or persistence, or amyloid lipid interactions. In some aspects, the term includes conditions characterized by abnormal protein folding or aggregation or amyloid formation, deposition, accumulation or persistence. In particular aspects, the disease is a condition of the central or peripheral nervous system or systemic organ. In more particular aspects the terms include conditions associated with the formation, deposition, accumulation, or persistence of amyloid or amyloid fibrils, comprising an amyloid protein comprising or selected from the group consisting of Aβ amyloid, AA amyloid, AL amyloid, IAPP amyloid, PrP amyloid, α₂-microglobulin amyloid, transthyretin, prealbumin, and procalcitonin, especially Aβ amyloid and IAPP amyloid. A disorder and/or disease may be a condition where it is desirable to dissociate abnormally aggregated proteins and/or dissolve or disrupt pre-formed or pre-deposited amyloid or amyloid fibril.

In certain aspects of the invention the disease is an amyloidosis. “Amyloidosis” refers to a diverse group of diseases of acquired or hereditary origin and characterized by the accumulation of one of several different types of protein fibrils with similar properties called amyloid. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta amyloid peptide (β-AP) are characteristic of Alzheimer's disease (both familial and sporadic forms); islet amyloid protein peptide (IAPP; amylin) is characteristic of the fibrils in pancreatic islet cell amyloid deposits associated with type II diabetes; and, β-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein (designated as AScr ro PrP-27).

Certain disorders are considered to be primary amyloidoses in which there is no evidence for preexisting or coexisting disease. Primary amyloidoses are typically characterized by the presence of “amyloid light chain-type” (AL-type) protein fibrils. In secondary amyloidosis there is an underlying chronic inflammatory or infectious disease state (e.g., rheumatoid arthritis, juvenile chronic arthritis, ankylosing spondylitis, psoriasis, Reiter's syndrome, Adult Still's disease, Behcet's Syndrome, Crohn's disease, chronic microbial infections such as osteomyelitis, tuberculosis, and leprosy, malignant neoplasms such as Hodgkin's lymphoma, renal carcinoma, carcinomas of the gut, lung, and urogenital tract, basel cell carcinoma, and hairy cell carcinoma). Secondary amyloidosis is characterized by deposition of AA type fibrils derived from serum amyloid A protein (ApoSSA). Heredofamilial amyloidoses may have associated neuropathic, renal, or cardiovascular deposits of the ATTR transthyretin type, and they include other syndromes having different amyloid components (e.g., familial Mediterranean fever which is characterized by AA fibrils). Other forms of amyloidosis include local forms, characterized by focal, often tumor-like deposits that occur in isolated organs. In addition, amyloidoses are associated with aging, and are commonly characterized by plaque formation in the heart or brain. Amyloidoses includes systemic diseases such as adult-onset disabetes, complications from long-term hemodialysis and consequences of chronic inflammation or plasma cell dyscrasias.

Amyloid diseases that can be treated and/or prevented using the compounds, compositions and methods of the invention include without limitation, Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, nephropathy with urticaria and deafness (Muckle—Wells syndrome), amyloidosis associated with systemic inflammatory diseases, idiopathic primary amyloidosis associated with myeloma or macroglobulinemia; amyloidosis associated with immunocyte dyscrasia; monoclonal gammopathy; occult dyscrasia; local nodular amyloidosis associated with chronic inflammatory diseases; amyloidosis associated with several immunocyte dyscrasias; familial amyloid polyneuropathy; hereditary cerebral hemorrhage with amyloidosis Alzheimer's disease and other neurodegenerative diseases, amyloidosis associated with chronic hemodialysis, diabetes type II, insulinoma, the amyloidosis of the prion diseases, (transmissible spongiform encephalopathies prion diseases), Creutzfeldt-Jakob disease, Gerstmann-Straussler syndrome, Kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In aspects of the invention, disorders and/or diseases include conditions associated with the formation, deposition, accumulation, or persistence of amyloid fibrils, especially the fibrils of an amyloid protein selected from the group consisting of Aβ amyloid, AA amyloid, AL amyloid, IAPP amyloid, PrP amyloid, α₂-microglobulin amyloid, transthyretin, prealbumin, and procalcitonin, especially Aβ amyloid and IAPP amyloid. Examples of such diseases include Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In other aspects of the invention, disorders and/or diseases that can be treated and/or prevented using the compounds, compositions and methods of the invention include conditions of the central or peripheral nervous system or a systemic organ that result in the deposition of proteins, protein fragments, and peptides in beta-pleated sheets, fibrils, and/or aggregates or oligomers. In particular the disease is Alzheimer's disease, presenile and senile forms; amyloid angiopathy; mild cognitive impairment; Alzheimer's disease-related dementia (e.g., vascular or Alzheimer dementia); tauopathy (e.g., argyrophilic grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Prion related disease (Creutzfeldt-Jakob disease, Gerstmann-Straussler-Scheinker disease, and variant Creutzfeldt-Jakob disease); Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; chronic infections and inflammations; and Type II Diabetes Mellitus associated with islet amyloid polypeptide (IAPP).

In aspects of the invention, in particular combination therapies, the disorder and/or disease is a neuronal disorder (e.g., Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction and dementia).

The compounds of the invention may also act to inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Examples of synuclein diseases or synucleinopathies suitable for treatment with a compound or composition of the invention are diseases associated with the formation, deposition, accumulation, or persistence of synuclein fibrils, especially α-synuclein fibrils, including without limitation Parkinson's disease, familial Parkinson's disease, Lewy body disease, the Lewy body variant of Alzheimer's disease, dementia with Lewy bodies, multiple system atrophy, olivopontocerebellar atrophy, neurodegeneration with brain iron accumulation type I, olfactory dysfunction, and the Parkinsonism-dementia complex of Guam.

In aspects of the invention, the disease is a Motor Neuron Disease associated with filaments and aggregates of neurofilaments and/or superoxide dismutase proteins, the Spastic paraplegia associated with defective function of chaperones and/or triple A proteins, or a spinocerebellar ataxia such as DRPLA or Machado-Joseph Disease.

In other aspects of the invention, the disease is a Prion Disease including Creutzfeldt-Jakob disease, Gerstmann-Strausller-Scheinfer disease, and variant Creutzfeldt-Jakob disease and a Amyloid Polyneuropathy including senile amyloid polyneuropathy or systemic amyloidosis.

In embodiments of the invention, the disease is Alzheimer's disease or Parkinson's disease including familial and non-familial types.

Alzheimer's disease (AD) affects about 4.5 million men and women in the United States alone. The incidence of Alzheimer's disease increases with age, and it affects up to 50 percent of people older than 85, with the risk generally increasing with age. Thus, one of the risk factors to consider when assessing whether a patient or patient population is a suitable host for the treatment and/or prevention of Alzheimer's disease is age. Of course signs or symptoms of the disease are an even better predictor. However, in many cases in people with Alzheimer's disease, changes in the brain may begin 10 to 20 years before any visible signs of dementia or symptoms appear. Thus, early treatment, even before the onset of visible signs, would positively affect the treatment and/or prevention of Alzheimer's disease, or would at least delay the effects thereof, or decrease their severity.

There are many diagnostic tests available to practitioners that help assess a patient's chance of having and/or developing Alzheimer's disease. These tests include, for example, tests known in the field as Mini-mental State Examination (MMSE), Clock Drawing Test, Clinical Dementia Rating (CDR) scale, Mini-Mental State Examination (MMSE), Functional Assessment, e.g., using a Functional Assessment Staging (FAST) scale, Alzheimer's Disease Assessment Scale-Cognitive Subscale (ADAS-Cog), among other tests known in the art. Many tests focus on assessment of memory, problem-solving, vision-motor coordination, attention, and abstract thinking, such as performing simple calculations in one's head. Doctors use a variety of assessments and laboratory measurements to make a diagnosis.

Brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles, are generally found in several areas of the human brain important for memory and cognitive function in patients with AD. Smaller numbers of these lesions in a more restricted anatomical distribution are also found in the brains of most aged humans who do not have clinical AD. Amyloid plaques and amyloid angiopathy also characterize the brains of individuals with Trisomy 21 (Down's Syndrome) and Hereditary Cerebral Hemorrhage with Amyloidosis of the Dutch-Type (HCHWA-D). Detection of such lesions, using MRI, CT, PET, SPECT, etc., is also useful in diagnosing AD.

In certain aspects of the invention, the disease may be characterized by an inflammatory process due to the presence of macrophages by an amyloidogenic protein or peptide. A method of the invention may involve inhibiting macrophage activation and/or inhibiting an inflammatory process. A method may comprise decreasing, slowing, ameliorating, or reversing the course or degree of macrophage invasion or inflammation in a patient.

A disease may be a condition that is associated with a molecular interaction that can be disrupted or dissociated with a compound of the invention. “A molecular interaction that can be disrupted or dissociated with a compound of the invention” includes an interaction comprising an amyloid protein and a protein or glycoprotein. An interaction comprising an amyloid protein includes an amyloid protein-amyloid protein interaction, amyloid-proteoglycan interaction, amyloid-proteoglycan/glycosaminoglycan (GAG) interaction and/or amyloid protein-glycosaminoglycan interaction. An interacting protein may be a cell surface, secreted or extracellular protein.

A disease that may be treated or prevented using a compound or composition of the invention includes a disease that would benefit from the disruption or dissolution of a molecular interaction comprising an amyloid protein and an interacting compound including a protein or glycoprotein. Examples of diseases that may be treated or prevented using a compound or composition of the invention include infectious diseases caused by bacteria, viruses, prions and fungi. Examples of such disorders and/or diseases are those associated with pathogens including Herpes simplex virus, Pseudorabies virus, human cytomegalovirus, human immunodeficiency virus, Bordetella pertussis, Chlamydia trachomatis, Haemophilus influenzae, Helicobacter pylori, Borrelia burgdorferi, Neisseria gonorrhoeae, Mycobacterium tuberculosis, Staphylococcus aureus, Streptococcus mutans, Streptococcus suis, Plasmodium falciparum, Leishmania amazonensi, Trypanozoma cruzi, Listeria monocytogenes, Mycoplasma pneumoniae, enterotoxigenic E. coli, uropathogenic E. coli, and Pseudomonas aeruginosa.

As used herein “mgA” or “milligrams of active” in reference to a cyclohexane polyalcohol compound refers to the amount of active cyclohexanehexyl polyalchol compound.

The unit “kg” as used herein in mg/kg or mgA/hr/kg refers to kilograms of body weight for a subject, preferably a mammal.

“Cₘₐₓ” refers to the maximum concentration in a use environment of a cyclohexane polyalcohol compound produced by the administration of a formulation or dosage form of the invention or by a method of the invention. The term “Cₘₐₓ” is synonymis with “peak levels”.

“Cₘᵢₙ” refers to the minimum concentration in a use environment of a cyclohexane polyalcohol compound produced by the administration of a formulation or dosage form of the invention or by a method of the invention. The term “Cₘᵢₙ” is synonymis with “trough levels”.

“IR” means immediate release.

“tₘₐₓ” refers to time to maximum observed concentration produced by the administration of a cyclohexane polyalcohol compound.

“Total blood drug exposure” refers to the area under the curve (“AUC”) determined by plotting the concentration of drug in the plasma (Y-axis) versus time (X-axis). AUC is generally an average value, and would, for example, be averaged over all the subjects in a study. The determination of AUCs is a well known procedure, and is described, for example, in “Pharmacokinetics; Processes and Mathematics”, by Peter Welling (ACS Monograph 185, Amer. Chem. Soc., Wash. D.C.: 1986).

“bid” refers to the administration of a formulation or dosage form twice during a 24 hour period.

“qd” refers to the administration of a formulation or dosage form once during a 24 hour period.

“Rate of release” or “release rate” of a compound means the quantity of compound released from a formulation or dosage form per unit time, e.g., milligrams of active drug released per hour (mgA/hr). Release rates for dosage forms are generally measured as an in vitro rate of dissolution, i.e., a quantity of compound released from the dosage form per unit time measured under appropriate conditions and in a suitable fluid. For example, dissolution tests can be performed and an in vitro dissolution profile can be prepared using methods known in the art.

An “in vitro dissolution profile” refers to a dissolution test in which the total amount of cyclohexane polyalcohol compound released is measured using a conventional U.S. Pharmacopeia (USP) apparatus for dissolution testing. See the USP apparatus described in United States Pharmacopoeia XXIII (USP) Dissolution Test Chapter 711, Apparatus 2 or 3. In an aspect the USP apparatus is an USP-2 apparatus containing 900 ml of an acetate buffer at pH4.0 and containing NaCl in a concentration of 0.75M at 37±0.5° C. If a dosage form is a sustained release tablet or non-disintegrating sustained release capsule, the USP apparatus is generally equipped with a paddle stirring at about 50 rpm. If a dosage form is multiparticulate and is not a tablet the USP apparatus is generally equipped with a paddle stirring at about 100 rpm. Thus, in an aspect where a sustained release dosage form is multiparticulate, the USP apparatus, or example is a Type 2 apparatus (paddle) at 100 rpm, a temperature of 37±0.5° C., a test solution of 900 ml of 0.05 M phosphate buffer containing 75 mM sodium laurel sulphate (pH 5.5).

In vitro dissolution profiles are routinely used in the manufacture of pharmaceuticals. Dissolution profiles can be developed using the procedures outlined by the FDA at www.usfda.gov and in United States Pharmacopeia (USP) Vol. 23, pp 1791-1793 (1995). A formulation or dosage form that meets the dissolution parameters disclosed herein may provide beneficial pharmacokinetic profiles.

A “dosage form” refers to a composition or device comprising a cyclohexane polyalcohol compound and optionally pharmaceutically acceptable carrier(s), excipient(s), or vehicles. A dosage form may be an immediate release dosage form or a sustained release dosage form.

An “immediate release dosage form” refers to a dosage form which does not include a component for sustained release i.e., a component for slowing disintegration or dissolution of an active compound.

These dosage forms generally rely on the composition of the drug matrix to effect the rapid release of the active ingredient agent By “sustained release dosage form”, also referred to as “extended release dosage form” is meant a dosage form that releases active compound over a number of hours. In an aspect, a sustained dosage form includes a component for slowing disintegration or dissolution of the active compound. In embodiments of the invention, a dosage form may be sustained release, engineered with or without an initial delay period. A sustained release dosage form may exhibit Tₘₐₓ values of at least two, four, six, or eight hours or more and preferably up to about 48 hours or more, for once per daily (qd) or twice per day (bid) dosing. Sustained release dosage forms may continuously release drug for sustained periods of at least about 4 to 6 hours or more, preferably about 8 hours or more and, in particular embodiments, about 12 hours or more, about 12 hours to 24 hours, or about 20 hours to 24 hours.

A sustained release dosage form can be formulated into a variety of physical structures or forms, including without limitation, tablets, lozenges, gelcaps, buccal patches, suspensions, solutions, gels, etc. In aspects of the invention the sustained release form results in administration of a minimum number of daily doses, in particular one, two or three daily doses, more particularly two daily doses (i.e., bid).

The term “zero-release profile” or “near zero release profile” means a substantially flat or unchanging amount of a particular drug in an environment of use (e.g, plasma, brain or CSF) in a patient over a particular time interval. By contrast, in most drug formulations the rate of drug release increases rapidly, followed by an exponentially declining rate of release. This type of drug release is categorized as the first order release.

The term “square root of time release profile” refers to the case where the cumulative release of drug released is proportional to the square root of time.

In aspects of the invention the zero-release profile will vary by no more than about 30%, 20%, 10%, or 5% from one time interval to the subsequent time interval. In aspects of the invention where the compound is administered at least twice a day, the zero-release profile will vary by no more than about 30%, 20%, 10%, or 5% from one time point to a subsequent time point of administration during the dosing period.

The term “zero order release rate” means a substantially constant release rate, such that the drug dissolves in the target environment of use at a substantially constant rate. More particularly, the rate of release of drug as a function of time varies by less than about 30%, preferably, less than about 20%, more preferably, less than about 10%, most preferably, less than about 5%, wherein the measurement is taken over the period of time wherein the cumulative release is between preferably, between or from about 25% and about 90% by total weight of the drug in the dosage form.

“Multiparticulate” refers to a plurality of particles wherein each particle is designed to yield sustained release of a cyclohexane polyalcohol compound. In aspects of the invention, each particle in a multiparticulate constitutes a self-contained unit of sustained release. In other aspects, the particles are formed into larger units. Multiparticulate particles preferably each comprise cyclohexane polyalcohol compounds and one or more excipients as needed for fabrication and performance. Individual particles may generally be between or from about 40 micrometers and about 5 mm, for example between or from about 50 mm and about 3 mm, or as another example between or from about 50 mm and about 1 mm, or as another example between or from about 50 mm and about 300 mm. Multiparticulates composed predominantly of particles in the low end of the size ranges are generally referred to as a powder. Multiparticulates composed predominantly of particles toward the high end of the size ranges are generally referred to as beads. Dosage forms comprising multiparticulates include unit dose packets or sachets and powders for oral suspension. Multiparticulates can be coated with controlled release polymers to achieve the release profile that will provide a therapeutic benefit.

A “matrix system”, refers to a dosage form where the drug is admixed with excipients, often in compressed or extruded form, such that the release of the drug from the dosage form is controlled by a combination of erosion and diffusion. Control of drug delivery by erosion involves the slow removal of the matrix material after administration to gradually expose and release the drug from the matrix. Control of drug delivery by diffusion involves the diffusion of soluble drug through the matrix excipients in a controlled manner. A matrix system may be hydrophilic or hydrophobic. Examples of matrix systems are described in US Published Application No. 2003/0180360 and International Published Application No. WO05102272.

### Formulations/Dosage Forms

The effectiveness of pharmaceutical compounds in the prevention and treatment of disease states depends on a variety of factors including the rate and duration of delivery of the compound from the dosage form into the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective therapies.

The invention provides dosage forms, formulations, and methods that provide advantages, in particular beneficial pharmacokinetic profiles, more particularly sustained pharmacokinetic profiles. A cyclohexane polyalcohol compound can be employed in dosage forms of this invention in pure or substantially pure form, in the form of its pharmaceutically acceptable salts, and also in other forms including anhydrous or hydrated forms. All such forms can be used within the scope of this invention.

In embodiments of the invention, a cyclohexane polyalcohol compound can include a pharmaceutically acceptable co-crystal, a co-crystal salt, polymorph, solvate, derivative, or a mixture thereof. A pharmaceutically acceptable co-crystal means a co-crystal that is suitable for use in contact with the tissues of a subject or patient without undue toxicity, irritation, allergic response and has the desired pharmacokinetic properties.

The term “co-crystal” as used herein means a crystalline material comprised of two or more unique solids at room temperature, each containing distinctive physical characteristics, such as structure, melting point, and heats of fusion. Co-crystals can be formed by an active pharmaceutical ingredient (API) and a co-crystal former either by hydrogen bonding or other non-covalent interactions, such as pi stacking and van der Waals interactions. An alternative embodiment provides for a co-crystal wherein the co-crystal former is a second API. In another embodiment, the co-crystal former is not an API. In another embodiment, the co-crystal comprises more than one co-crystal former. For example, two, three, four, five, or more co-crystal formers can be incorporated in a co-crystal with an API. Pharmaceutically acceptable co-crystals are described, for example, in “Pharmaceutical co-crystals,” Journal of Pharmaceutical Sciences, Volume 95 (3) Pages 499-516, 2006. The methods producing co-crystals are discussed in the United States Patent Application 20070026078.

A co-crystal former which also must be a pharmaceutically acceptable compound, may be, for example, benzoquinone, terephthalaldehyde, saccharin, nicotinamide, acetic acid, formic acid, butyric acid, trimesic acid, 5-nitroisophthalic acid, adamantane-1,3,5,7-tetracarboxylic acid, formamide, succinic acid, fumaric acid, tartaric acid, malic acid, tartaric acid, malonic acid, benzamide, mandelic acid, glycolic acid, fumaric acid, maleic acid, urea, nicotinic acid, piperazine, p-phthalaldehyde, 2,6-pyridinecarboxylic acid, 5-nitroisophthalic acid, citric acid, and the alkane- and arene-sulfonic acids such as methanesulfonic acid and benezenesulfonic acid.

In each process according to the invention, there is a need to intimately combine the API with the co-crystal former, involving grinding the two solids together or melting one or both components and allowing them to recrystallize. This may also involve either solubilizing the API and adding the co-crystal former, or solubilizing the co-crystal former and adding the API. Crystallization conditions are applied to the API and co-crystal former. This may entail altering a property of the solution, such as pH or temperature and may require concentration of the solute, usually by removal of the solvent, typically by drying the solution. Solvent removal results in the concentration of both API and co-crystal former increasing over time so as to facilitate crystallization.

A beneficial pharmacokinetic profile, in particular a sustained pharmacokinetic profile, may be obtained by the administration of a formulation or dosage form suitable for once or twice a day, preferably once a day, administration comprising one or more cyclohexane polyalcohol compound present in an amount sufficient to provide the required concentration or dose of the compound to an environment of use to treat a disorder and/or disease disclosed herein. In an aspect, the environment of use is the brain, in particular extracellular or interstitial brain tissue. In an aspect, the environment of use is plasma and/or CSF.

A beneficial pharmacokinetic profile, in particular a sustained pharmacokinetic profile, may be obtained by the administration of a formulation or dosage form suitable for once or twice a day, preferably once a day administration comprising one or more cyclohexane polyalcohol compound present in an amount sufficient to provide the required plasma brain, or CSF concentration or dose (e.g. daily dose) of the compound to treat a disorder and/or disease disclosed herein.

In an aspect, the concentration of a cyclohexane polyalcohol compound in CSF, brain, or plasma is at least about 0.05 μM to at least about 125 μM.

In embodiments of dosage forms of the invention, the concentration of the compound in CSF, brain or plasma is between or from about 0.05 μM to 100 μM, 0.05 μM to 90 μM, 0.05 μM to 80 μM, 0.05 μM to 70 μM, 0.05 μM to 60 μM, 0.05 μM to 50 μM, 0.05 μM to 40 μM, 0.05 μM to 30 μM, or 0.05 μM to 20 μM.

In other embodiments of dosage forms of the invention, the concentration of the compound in CSF, brain or plasma is between or from about 0.1 μM to 100 μM, 0.1 μM to 90 μM, 0.1 μM to 80 μM, 0.1 μM to 70 μM, 0.1 μM to 60 μM, 0.1 μM to 50 μM, 0.1 μM to 40 μM, 0.1 μM to 30 μM, 0.1 μM to 20 μM, or 0.1 μM to 10 μM.

In further embodiments of dosage forms of the invention, the concentration of the compound in CSF, brain, or plasma is between or from about 0.125 to 50 μM, 0.125 to 50 μM, 0.125 to 40 μM, 0.125 to 30 μM, 0.125 to 20 μM, or 0.125 to 10 μM.

In embodiments of dosage forms of the invention, the concentration of the compound in CSF, brain, or plasma is between or from about 0.5 to 100 μM, 0.5 to 50 μM, 0.5 to 40 μM, 0.5 to 30 μM, 0.5 to 20 μM, or 0.5 to 10 μM.

In embodiments of dosage forms of the invention, the concentration of the compound in CSF, brain, or plasma is between or from about 0.8 to 100 μM, 0.8 to 50 μM, 0.8 to 40 μM, 0.8 to 30 μM, 0.8 to 20 μM, or 0.8 to 10 μM.

In embodiments of dosage forms of the invention, the concentration of the compound in CSF, brain, or plasma is between or from about 0.9 to 50 μM, 0.9 to 40 μM, 0.9 to 30 μM, 0.9 to 20 μM, or 0.9 to 10 μM.

In embodiments of dosage forms of the invention, the concentration of the compound in CSF, brain, or plasma is between or from about 1 to 50 μM, 1 to 40 μM, 1 to 30 μM, 1 to 20 μM, 1 to 10 μM, or 1 μM to 5 μM.

In embodiments of dosage forms of the invention, the concentration of the compound in CSF, brain, or plasma is between or from about, 1.25 to 50 μM. 1.25 to 40 μM, 1.25 to 30 μM, 1.25 to 20 μM, 1.25 to 10 μM, or 1.25 to 5 μM.

In particular embodiments, the concentration in CSF, brain, or plasma is between or from about 1 to 50 μM, 1 to 20 μM, 1 to 10 μM, 1 to 6 μM or 1 to 5 μM.

In other particular embodiments, the concentration in CSF, brain, or plasma is between or from about 2 to 6 μM, 3 to 6 μM, or 4 to 6 μM, or about 5 μM.

In an aspect, the required dose of a cyclohexane polyalcohol compound administered once, twice, or three times or more daily is about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In an aspect, the required dose of a cyclohexane polyalcohol compound administered once or twice, daily, especially once, is about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In embodiments of the invention, the required dose administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg. In embodiments of the invention, the required daily dose is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

A beneficial pharmacokinetic profile can be obtained by the administration of a formulation or dosage form suitable for once or twice a day administration, preferably twice a day administration comprising one or more cyclohexane polyalcohol compound present in an amount sufficient to provide the required dose of the compound. In an aspect, the required dose of the compound administered once or twice daily is about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg.

In embodiments of the invention, the required dose administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg.

In other embodiments of the invention, the required daily dose is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

In aspects of the invention, dosage forms and formulations are provided that minimize the variation between peak and trough plasma and/or cerebral spinal fluid levels of cyclohexane polyalcohol compounds (e.g., scyllo-cyclohexanehexyl compounds or epi-cyclohexanehexyl compounds), and in particular provide a sustained therapeutically effective amount of cyclohexane polyalcohol compounds.

Aspects of the invention relate to a formulation comprising amounts of one or more cyclohexane polyalcohol compound (e.g., scyllo-cyclohexanehexyl compound or epi-cyclohexanehexyl compound) that result in therapeutically effective amounts of the compound over a dosing period, in particular a 24 hour dosing period. In an embodiment the therapeutically effective amounts of a cyclohexane polyalcohol compound are between or from about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg. In a particular aspect, the therapeutic amounts for twice daily administration are between or from about 1 to 50 mg/kg, 1 to 40 mg/kg, 2.5 to 40 mg/kg, 3 to 40 mg/kg, 3 to 35 mg/kg, most preferably 3 to 30 mg/kg. In an embodiment, the therapeutically effective amounts of a cyclohexane polyalcohol compound administered twice daily are between or from about 3 to 30 mg/kg administered bid. In another embodiment, the therapeutically effective amounts of a cyclohexane polyalcohol compound administered daily are between or from about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

Further aspects of the invention relate to a unit dose formulation for once or twice a day administration comprising one or more cyclohexane polyalcohol compound (e.g., scyllo-cyclohexanehexyl compound or epi-cyclohexanehexyl compound) that provides peak concentrations of the compound, Cₘₐₓ, that are not statistically significantly different from those obtained with a dosage form administered more than twice per day (over a 24 hour period).

Embodiments of the invention relate to a dosage form comprising one or more cyclohexane polyalcohol compound (e.g., scyllo-cyclohexanehexyl compound or epi-cyclohexanehexyl compound) that provides peak plasma concentrations of the compound, Cₘₐₓ, of from or between about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml. In embodiments, the Cₘₐₓ, is between or from about 1-125 μg/ml, 1-100 μg/ml, 5-70 μg/ml, 5-50 μg/ml, 10-100 μg/ml, 10-90 μg/ml, 10-80 μg/ml, 10-70 μg/ml, 10-60 μg/ml, 10-50 μg/ml or 10-40 μg/ml. In particular embodiments, the Cₘₐₓ is from or between about 5 to 70 μg/ml, 5 to 65 μg/ml, 5 to 50 μg/ml, 5 to 40 μg/ml, 5 to 30 μg/ml, or 5 to 20 μg/ml.

Embodiments of the invention relate to a dosage form comprising one or more cyclohexane polyalcohol compound (e.g., scyllo-cyclohexanehexyl compound or epi-cyclohexanehexyl compound) that provides peak CSF concentrations of the compound, Cₘₐₓ, that are about 20-80%, 25-75%, 25-70%, 25-65%, or 30-65%, preferably about 30-60% of peak plasma concentrations following administration.

Embodiments of the invention relate to a dosage form comprising one or more cyclohexane polyalcohol compound (e.g., scyllo-cyclohexanehexyl compound or epi-cyclohexanehexyl compound) that provides peak CSF or brain concentrations of the compound, Cₘₐₓ, of between or from about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml. In particular embodiments, the Cₘₐₓ is between or from about 5 to 70 μg/ml, 5 to 65 μg/ml, 5 to 50 μg/ml, 5 to 40 μg/ml, 5 to 30 μg/ml, or 5 to 20 μg/ml. In other particular embodiments, the dose of the compound provides a peak CSF concentration of the compound, Cₘₐₓ, between or from about 1 to 75 μg/ml, 1-70 μg/ml, 1 to 60 μg/ml, 1-55 μg/ml, 1-50 μg/ml, 1-30 μg/ml, 1-25 μg/ml, 1-20 μg/ml, or 1-15 μg/ml.

In further aspects, the invention relates to a formulation or dosage form for once or twice a day administration comprising one or more cyclohexane polyalcohol compound (e.g., scyllo-cyclohexanehexyl compound or epi-cyclohexanehexyl compound) that provides an extent of absorption, as defined by area under the curve (AUC) equivalent to those produced by three or more a day dosage forms of the compounds. In a particular aspect, the AUC, in particular the AUC_{0-inf}, is between or from about 20 to 600 μg·h/ml, 50 to 600 μg·h/ml, 100 to 600 μg·h/ml, 100 to 300 μg·h/ml, or 100 to 250 μg·h/ml, 15 to 125 μg·h/ml, 20 to 135 μg·h/ml, 80-270 μg·h/ml, 80-200 μg·h/ml, 80-150 μg·h/ml, 80-125 μg·h/ml, or 80-100 μg·h/ml.

Further aspects of the invention relate to a formulation or dosage form comprising one or more cyclohexane polyalcohol compound (e.g., scyllo-cyclohexanehexyl compound or epi-cyclohexanehexyl compound) that provides an AUC for plasma of about 20 to 600 μg·h/ml, 50 to 600 μg·h/ml, 100 to 600 μg·h/ml, 100 to 300 μg·h/ml, or 100 to 250 μg·h/ml, 15 to 125 μg·h/ml, or 20 to 135 μg·h/ml, 80-270 μg·h/ml, 80-200 μg·h/ml, 80-150 μg·h/ml, 80-125 μg·h/ml, or 80-100 μg·h/ml.

Still further aspects of the invention relate to a formulation or dosage form comprising one or more cyclohexane polyalcohol compound (e.g., scyllo-cyclohexanehexyl compound or epi-cyclohexanehexyl compound) that provides an AUC for CSF of about 40-75%, 45-70%, 50-70%, 55-70%, 55-65%, or 60-65%, preferably 30-60%, of the AUC for plasma levels.

Other aspects of the invention relate to a formulation or dosage form for once or twice a day administration comprising one or more cyclohexane polyalcohol compound (e.g., scyllo-cyclohexanehexyl compound) that provides minimum concentrations of the compound, Cₘᵢₙ, that are not statistically significantly different from those obtained with a dosage form administered more than twice a day (over a 24 hour period).

In further aspects, the invention provides a formulation or dosage form comprising one or more cyclohexane polyalcohol compound (e.g., scyllo-cyclohexanehexyl compound) that provides an elimination t_{1/2} of 1 to 100 hours, 1 to 80 hours, 1 to 70 hours, 1 to 50 hours, 1 to 42 hours, 1 to 33 hours or 3 to 50, 16 to 32, 5 to 30 hours, 10 to 30 hours, 1 to 28 hours, 1 to 25 hours, 10 to 25 hours, 1 to 24 hours, 10 to 24 hours, 13 to 24 hours, 1 to 23 hours, 1 to 20 hours, 1 to 18 hours, 1 to 15 hours, 1 to 14 hours, 1 to 13 hours, 1 to 12 hours, 1 to 10 hours, 1 to 8 hours, 1 to 7 hours, 1 to 5 hours, 1 to 4 hours, 1 to 3 hours or 3 to 5 hours.

In further aspects, the invention provides a twice daily dosage form comprising one or more cyclohexane polyalcohol compound (e.g., scyllo-cyclohexanehexyl compound) that has a relative bioavailability, as measured by AUCₒₘₜₕ of at least 50%, 60%, 65%, 70%, 75%, 80%, 85%, or 90% of the bioavailability of a single daily dosage form, preferably 70%, 75%, 80%, 85%, or 90% of the bioavailability of a single daily dosage form.

Dosage forms and formulations of the invention may provide for the release of a cyclohexane polyalcohol compound following zero-order kinetics i.e., the plasma, brain and/or CSF levels of the compound remain about constant throughout the delivery period, preferably above a selected Cₘᵢₙ. In an aspect, the dosage forms are for twice daily administration and the Cₘᵢₙ after the administration of the second dose is greater than the Cₘᵢₙ after the administration of the first dose. Accordingly, dosage forms, formulations, and methods may provide for zero-order release rate of a cyclohexane polyalcohol compound minimizing the variance between peak and trough levels of the compound in the plasma, brain or CSF. In an aspect, the invention provides a formulation or dosage form comprising one or more cyclohexane polyalcohol compound (e.g., scyllo-cyclohexanehexyl compound) that produces a zero-order release profile thus producing essentially flat plasma, brain or CSF levels of the compound once steady-state levels have been achieved.

A zero-order or near zero-order release dosage form of the invention may allow a reduction in dosing frequency improving the dosage compliance on the part of subjects.

The invention relates to a dosage form comprising a cyclohexane polyalcohol compound, for administration at a first time point and a second time point over a dosing period, wherein the dosage form comprises a dose of compound sufficient to provide a beneficial pharmacokinetic profile whereby the concentration or peak concentration of compound in plasma, brain or CSF does not significantly vary during the dosing period. In an aspect, the total dosing period is about 8, 12, 18, 20 24, or 48 hours. In embodiments of this aspect, the second time point is about 4 to 20 hours, 4 to 18 hours, 4 to 12 hours, 4 to 14 hours, in particular 6 to 14, 6 to 12, 6 to 8, 8 to 12, or 8 to 10 hours following the first time point. In another aspect, the administration of the compound at the second time point results in concentrations or peak concentrations of the compound in plasma, brain or CSF that do not vary by more than 90%, 80%, 70%, 60%, 50%, 30%, 20%, 15%, 20%, 5%, or 3% from the concentration or peak concentration of the compound in plasma, brain or CSF following the first time point. In an aspect, the beneficial pharmacokinetic profile is a zero order release profile which does not vary by more than about 30%, 20%, 10%, or 5% from the first time point to the second time point of administration. In an aspect, the zero order release profile does not vary by more than about 20%, 10%, or 5% from the first time point to a third time point which is at least 2, 4, 6, 8, 10, 12, 14, or 16 hours following the second time point. In other aspects, the compound is a scyllo-cylcohexanehexyl compound. In particular aspects, the dose of the compound is between or from about 1 to 100 mg/kg, 1 to 90 mg/kg, 1 to 80 mg/kg, 1 to 70 mg/kg, 1 to 60 mg/kg, 1 to 50 mg/kg, 1 to 40 mg/kg, 2.5 to 40 mg/kg, 3 to 40 mg/kg, 3 to 35 mg/kg, or 3 to 30 mg/kg.

The invention relates to a dosage form comprising a cyclohexane polyalcohol compound, for administration to a subject at a first time point and a second time point over a dosing period, wherein the dosage form comprises a dose of compound sufficient to provide a Cₘₘ, in plasma, brain or CSF after the second time point greater than the Cₘᵢₙ after the first time point. In an aspect, the C after the second time point is 5%, 10%, 15%, 20%, 25%, 30%, 35%, 40%, 45%, 50%, 60%, 70%, 80%, or 90% greater than the Cₘᵢₙ after the first time point. In an aspect, the total dosing period is about 8, 12, 18, 20, 24 or 48 hours. In embodiments of this aspect, the second time point is about 4 to 20 hours, 4 to 18 hours, 4 to 14 hours, 4 to 12 hours, in particular 6 to 14, 6 to 12, 6 to 8, 8 to 12, or 8 to 10 hours following the first time point. In particular aspects, the dose of the compound is between or form about 1 to 100 mg/kg, 1 to 90 mg/kg, 1 to 80 mg/kg, 1 to 70 mg/kg, 1 to 60 mg/kg, 1 to 50 mg/kg, 1 to 40 mg/kg, 2.5 to 40 mg/kg, 3 to 40 mg/kg, 3 to 35 mg/kg, or 3 to 30 mg/kg. In embodiments of this aspect of the invention, a cyclohexane polyalcohol compound can be periodically administered to the subject subsequent to the second time point, in particular 1, 2, 3, 4, 5, 6, 7, or more days following the second time point, to provide a Cₘᵢₙ in plasma, brain or CSF substantially the same as the Cₘᵢₙ after the first time point or after the second time point, preferably the Cₘᵢₙ after the second time point.

The invention relates to a dosage form comprising a cyclohexane polyalcohol compound, for administration to a subject at a first time point and a second time point over a dosing period, wherein the dosage form comprises a dose of compound sufficient to maintain a concentration of compound in the subject so that Cₘᵢₙ in plasma, brain or CSF after the second time point is greater than the Cₘᵢₙ after the first time point. In an aspect, the Cₘᵢₙ after the second time point is 5%, 10%, 15%, 20%, 25%, 30%, 35%, 40%, 45%, 50%, 60%, 70%, 80%, or 90% greater than the Cₘᵢₙ after the first time point. In an aspect, the total dosing period is about 8, 12, 18, 20, 24 or 48 hours. In embodiments of this aspect, the second time point is about 4 to 20 hours, 4 to 18 hours, 4 to 14 hours, 4 to 12 hours, in particular 6 to 14, 6 to 12, 6 to 8, 8 to 12 or 8 to 10 hours following the first time point. In particular aspects, the dose of the compound is between or from about 1 to 100 mg/kg, 1 to 90 mg/kg, 1 to 80 mg/kg, Ito 70 mg/kg, 1 to 60 mg/kg, 1 to 50 mg/kg, 1 to 40 mg/kg, 2.5 to 40 mg/kg, 3 to 40 mg/kg, 3 to 35 mg/kg, or 3 to 30 mg/kg. In embodiments of this aspect of the invention, a cyclohexane polyalcohol compound can be periodically administered to the subject subsequent to the second time point, in particular 1, 2, 3, 4, 5, 6, 7, or more days following the second time point, to provide a Cₘᵢₙ in plasma, brain or CSF substantially the same as the Cₘᵢₙ after the first time point or after the second time point, preferably the Cₘᵢₙ after the second time point.

The invention related to formulations or dosage forms with beneficial pharmacokinetic profiles obtained by administration of an oral formulation suitable for once a day or twice a day, administration, preferably twice a day administration, comprising a cyclohexane polyalcohol compound, in particular a scyllo-cyclohexanehexyl compound or epi-cyclohexanehexyl compound, typically present in an amount sufficient to provide the required plasma, brain and/or CSF drug concentrations, or required dose (e.g., daily dose) of a cyclohexane polyalcohol compound and so that the formulation exhibits a favourable or improved in vitro dissolution profile.

In an aspect, a formulation or dosage form exhibits the following in vitro dissolution profile:
a) from about 15% to about 30% of total compound is released after 3 hours of measurement;
b) from about 50% to about 70% of total compound is released after 9 hours of measurement;
c) from about 65% to about 95% of total compound is released after 12 hours of measurement; and,
d) at least 88% of total compound is released after 18 hours of measurement.

In another aspect, a formulation or dosage form exhibits the following in vitro dissolution profile:
a) from about 15% to about 25% of total compound is released after 3 hours of measurement in the apparatus;
b) from about 45% to about 69% of total compound is released after 9 hours of measurement in the apparatus;
c) from about 59% to about 90% of total compound is released after 12 hours of measurement in the apparatus; and,
d) at least 90% of total compound is released after 18 hours of measurement in the apparatus.

In an aspect, a formulation or dosage form exhibits the following in vitro dissolution profile:
a) from about 35% to about 50% of total compound is released after 3 hours of measurement;
b) from about 70% to about 90% of total compound is released after 9 hours of measurement;
c) from about 80% to about 90% of total compound is released after 12 hours of measurement; and,
d) at least 99% of total compound is released after 18 hours of measurement.

An aspect of the invention relates to a qd or bid dosage form that has a dissolution profile as disclosed herein. An oral dosage form of the invention may also produce total absorption of the cyclohexane polyalcohol compound, in particular scyllo-cyclohexanehexyl compound.

Another aspect of the invention provides a dosage form comprising a cyclohexane polyalcohol compound in an amount that provides a stoichiometric relationship of cyclohexane polyalcohol compound to amyloid peptide of about 40:1, 35:1, 30:1, 25:1, 20:1 or 15:1, preferably 25:1.

A dosage form or formulation of the invention may be an immediate release dosage form or a non-immediate release delivery system, including without limitation a delayed-release or sustained-release dosage form. Particularly, the dosage form or formulation may exhibit a delayed release followed by immediate release or sustained release.

Accordingly, this invention provides a sustained-release dosage form of a cyclohexane polyalcohol compound or a pharmaceutically acceptable salt thereof which advantageously achieves a more sustained drug plasma, brain or CSF level response while mitigating or eliminating drug concentration spikes by providing a substantially steady release of cyclohexane polyalcohol compound over time.

In an aspect, a sustained-release oral dosage form is provided comprising one or more cyclohexane polyalcohol compound, in particular a scyllo-cyclohexanehexyl compound, in an amount that provides release of the compound at a substantially constant release rate over a dosing period resulting in a substantially constant plasma concentration of the compound. The substantially constant plasma concentration preferably correlates with one or more therapeutic effects disclosed herein. In embodiments, the plasma concentration is between or from about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or to 20 μg/ml. In particular embodiments, the plasma concentration is between or from about 5 to 70 μg/ml, 5 to 65 μg/ml, 5 to 50 μg/ml, 5 to 40 μg/ml, 5 to 30 μg/ml, or 5 to 20 μg/ml.

In another aspect, a sustained-release oral dosage form is provided comprising one or more cyclohexane polyalcohol compound, in particular a scyllo-cyclohexanehexyl compound, in an amount that provides release of the compound at a substantially constant release rate over a dosing period resulting in a substantially constant brain or CSF concentration of the compound. The substantially constant CSF concentration preferably correlates with one or more therapeutic effects disclosed herein, i.e. substantially constant therapeutic effectiveness of the compounds over a prolonged therapy period.

In embodiments of a sustained-release oral dosage form, the dosage form provides a CSF concentration from or between about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μ/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml.

In embodiments of a sustained-release oral dosage form, the dosage form provides a concentration of the compound in the brain from or between about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml.

In particular embodiments of a sustained-release oral dosage form, the dosage form provides a Cₘₐₓ from or between about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml. In particular embodiments, the Cₘₐₓ is between or from about 5 to 70 μg/ml, 5 to 65 μg/ml, 5 to 50 μg/ml, 5 to 40 μg/ml, 5 to 30 μg/ml, or 5 to 20 μg/ml.

In another aspect this invention relates to a sustained release dosage form of a cyclohexanehexyl suitable for administration, such as an oral administration, to a subject, in particular a mammal, which results in a maximum cyclohexane polyalcohol compound CSF concentration, Cₘₐₓ, which is less than about 95%, 90%, 85%, 80% or 75% of the Cₘₐₓ determined when an equal dose of the compound is administered to the subject in the form of an immediate release dosage form.

In another aspect this invention provides a sustained release dosage form of a cyclohexanehexyl suitable for administration, such as an oral administration, to a subject, in particular a mammal, which results in a maximum cyclohexane polyalcohol compound plasma concentration, Cₘₐₓ, which is less than about 95%, 90%, 85%, 80% or 75% of the Cₘₐₓ determined when an equal dose of the compound is administered to the subject in the form of an immediate release formulation.

In an embodiment, the sustained release dosage form releases not more than about 70% or 80% by weight of the cyclohexane polyalcohol compound within the first hour following ingestion and releases the compound at a rate of at least 0.01 to 50 mgA/hr, 0.1 to 50 mgA/hr, 0.1 to 40 mgA/hr, 0.1 to 35 mgA/hr, 0.1 to 30 mgA/hr, 0.1 to 20 mgA/hr, 0.1 to 10 mgA/hr, 0.1 to 5 mgA/hr, 1 to 50 mgA/hr, 1 to 40 mgA/hr, 1 to 35 mgA/hr, 1 to 30 mgA/hr, 1 to 20 mgA/hr, 1 to 10 mgA/hr, 1 to 5 mgA/hr, 2 to 50 mgA/hr, 2 to 40 mgA/hr, 2 to 35 mgA/hr, 2 to 30 mgA/hr, 2 to 20 mgA/hr, 2 to 10 mgA/hr, 2 to 5 mgA/hr, 3 to 50 mgA/hr, 3 to 40 mgA/hr, 3 to 35 mgA/hr, 3 to 30 mgA/hr, 3 to 20 mgA/hr, 3 to 10 mgA/hr, 3 to 5 mgA/hr, 1 to 3 mgA/hr, 0.1 to 30 mgA/hr, preferably at a rate not exceeding 0.01, 0.1, 1, 2, 3, 4, 5, 10, 15, 20, 25, 30 or 35 mgA/hr, more preferably at a rate not exceeding 20, 25 or 30 mgA/hr.

Aspects of the invention relate to a dosage form that releases cyclohexane polyalcohol compound into a use environment (e.g, plasma, brain or CSF), provided the dosage form (1) releases not more than about 70%, 80%, or 90% by weight of the cyclohexane polyalcohol compound contained therein within the first hour following entry into a use environment and (2) releases cyclohexane polyalcohol compound at a rate of at least about 0.01 to 40 mgA/hr, 0.1 to 40 mgA/hr, 1 to 40 mgA/hr, 2 to 40 mgA/hr, 3 to 40 mgA/hr, 3 to 40 mgA/hr, 3 to 35 mgA/hr, 3 to 30 mgA/hr, 3 to 20 mgA/hr, 3 to 10 mgA/hr, 3 to 5 mgA/hr, 1 to 3 mgA/hr, 0.1 to 30 mgA/hr, preferably at a rate not exceeding 3, 5, 10, 15, 20, 25, 30 or 35 mgA/hr, more preferably at a rate not exceeding 20, 25 or 30 mgA/hr.

Low rates of cyclohexane polyalcohol compound release rates are within the scope of the invention particularly for low weight and/or elderly patients. Thus a cyclohexane polyalcohol compound release rate of about 1, 2, 3, 5, 10, 15, 20, 25, 30 or 35 mgA/hr after ingestion represents a profile within the scope of an embodiment of the invention. The rate can be sufficient to deliver a therapeutically sufficient amount of cyclohexane polyalcohol compound before the dosage form is cleared. Accordingly, in one embodiment, dosage forms according to the invention release cyclohexane polyalcohol compound at a rate of at least about 3, 5, 10, 15, 20, 25, or 30 mgA/hr.

This invention provides a sustained release dosage form of cyclohexane polyalcohol compound suitable for administration, such as oral administration to a subject, in particular a mammal, which results in a maximum cyclohexane polyalcohol compound plasma or CSF concentration, Cₘₐₓ, which is less than about 80% of the Cₘₐₓ determined when an equal dose of cyclohexane polyalcohol compound is administered to the mammal, in the form of an immediate release dosage form. In an embodiment, a sustained release dosage form (1) releases not more than about 70%, 80%, or 90% by weight of the cyclohexane polyalcohol compound contained therein within the first hour following ingestion and (2) releases cyclohexane polyalcohol compound at a rate of at least about 3, 5, 10, 15, 20, 25, 30 or 35 mgA/hr.

In an aspect, a sustained release cyclohexane polyalcohol compound dosage form according to the invention releases at least about 60%, 70%, 80%, or 90% by weight of its contained cyclohexane polyalcohol compound within 24 hours, preferably within 18 hours, most preferably within 16 hours, within 8 hours, or within 6 hours. In other aspects, a dosage form according to the invention releases substantially all of its cyclohexane polyalcohol compound well before 24 hours at a rate not exceeding about 3, 5, 10, 15, 20, 25, or 35 mgA/hr.

In an aspect, a controlled release cyclohexane polyalcohol compound twice daily dosage form according to the invention releases at least about 70%, 80%, or 90% by weight of their contained cyclohexane polyalcohol compound within 4 hours, preferably within 6 hours, most preferably within 8 hours.

In an embodiment, the invention provides a sustained release dosage form of a cyclohexane polyalcohol compound suitable for oral administration to a mammal, which results in a maximum cyclohexane polyalcohol compound plasma concentration, Cₘₐₓ, of about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml when administered as a once or twice daily dose, preferably twice daily dose. In particular embodiments, the Cₘₐₓ is between or from about 5 to 70 μg/ml, 5 to 65 μg/ml, 5 to 50 μg/ml, 5 to 40 μg/ml, 5 to 30 μg/ml, or 5 to 20 μg/ml.

In an embodiment, the invention provides a sustained release dosage form of a cyclohexane polyalcohol compound suitable for oral administration to a mammal, which results in a maximum cyclohexane polyalcohol compound CSF concentration, Cₘₐₓ, of about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml when administered as a once or twice daily dose, preferably twice daily dose.

In another embodiment, the invention provides a sustained release dosage form of a cyclohexane polyalcohol compound suitable for oral administration to a mammal, which results in a maximum cyclohexane polyalcohol compound plasma concentration, Cₘₐₓ, of about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml when administered as a single dose or twice daily dose, preferably a twice daily dose. In a particular embodiment, the cyclohexane polyalcohol compound is released over 4 to 12, 6 to 12 or 8 to 12 hours.

In another embodiment, the invention provides a sustained release dosage form of a cyclohexane polyalcohol compound suitable for oral administration to a mammal, which results in a maximum cyclohexane polyalcohol compound CSF concentration, Cₘₐₓ, of about 1 to 125 μg/ml, 1 to 100 μg/ml, 1 to 90 μg/ml, 1 to 80 μg/ml, 1 to 70 μg/ml, 1 to 60 μg/ml, 1 to 50 μg/ml, 1 to 40 μg/ml, 1 to 30 μg/ml, 1 to 20 μg/ml, 1 to 10 μg/ml, 1 to 5 μg/ml, 5 to 125 μg/ml, 5 to 100 μg/ml, 5 to 70 μg/ml, 5 to 50 μg/ml, 10 to 100 μg/ml, 10 to 90 μg/ml, 10 to 80 μg/ml, 10 to 70 μg/ml, 10 to 60 μg/ml, 10 to 50 μg/ml, 10 to 40 μg/ml, 10 to 30 μg/ml, or 10 to 20 μg/ml, when administered as a single dose or twice daily dose, preferably twice daily dose. In a particular embodiment, the cyclohexane polyalcohol compound is released over 4 to 12, 6 to 12 or 8 to 12 hours.

In a further embodiment, this invention provides a sustained release dosage form of cyclohexane polyalcohol compound suitable for oral administration to a mammal, which results in a maximum cyclohexane polyalcohol compound plasma concentration, Cₘₐₓ, of about 5 to about 125 μg/ml, 5 to about 100 μg/ml, 5 to about 70 μg/ml, 5 to about 50 μg/ml, 10 to about 120 μg/ml, 10 to about 100 μg/ml, 10 to about 90 μg/ml, 10 to about 80 μg/ml, 10 to about 70 μg/ml, 10 to about 50 μg/ml, or 10 to about 40 μg/ml 1, wherein plasma levels at Cₘₐₓ do not exceed two times the plasma level 24 hours after administration.

In aspects of the invention, a sustained release cyclohexane polyalcohol compound dosage forms provides a decreased Cₘₐₓ relative to the Cₘₐₓ for immediate-release dosage forms containing equal amounts of cyclohexane polyalcohol compound. In particular aspects, a sustained release dosage form exhibits a Cₘₐₓ which is less than or equal to about 70%, 75%, 80%, 85%, or 90% of the Cₘₐₓ provided by an equivalent amount of cyclohexane polyalcohol compound in an immediate release form.

Dosage forms of the invention can additionally provide a total blood drug exposure which, relative to an equivalent amount of cyclohexane polyalcohol compound in an immediate-release dosage form, is not proportionately decreased as much as the sustained release Cₘₐₓ. In an embodiment, a sustained release cyclohexane polyalcohol compound dosage form exhibits a Cₘₐₓ that is 50%, 55%, 60%, 65%, or 70% of the Cₘₐₓ produced by an immediate release cyclohexane polyalcohol compound dosage form, and exhibits an AUC that is higher than 60%, 65%, 70%, 75%, or 80% of that provided by the immediate release dosage form.

A dosage form or formulation may be in any form suitable for administration to a subject, including without limitation, a form suitable for oral, parenteral, intravenous (bolus or infusion), intraperitoneal, subcutaneous, or intramuscular administration. A dosage form or formulation may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder for inhibition of amyloid formation, deposition, accumulation, and/or persistence, regardless of its clinical setting.

In an aspect of the invention a dosage form or formulation is an oral dosage form or formulation including without limitation tablets, caplets, soft and hard gelatin capsules, pills, powders, granules, elixirs, tinctures, suspensions, syrups, and emulsions. In another aspect of the invention a dosage form or formulation is a parenteral dosage form including without limitation an active substance in a sterile aqueous or non-aqueous solvent, such as water, isotonic saline, isotonic glucose solution, buffer solution, or other solvents conveniently used for parenteral administration.

In aspects of the invention a dosage form is a tablet including compressed tablets, coated tablets, osmotic tablets, and other forms known in the art. In other aspects of the invention, the dosage form is a capsule well known in the art. In still other aspects of the invention, the dosage form is a pill which embraces small, round solid dosage forms that comprise microparticles mixed with a binder and other excipients. [See for example, the standard text, in Remington: The Science and Practice of Pharmacy (21^{st} Edition. 2005, University of the Sciences in Philadelphia (Editor), Mack Publishing Company), and in The United States Pharmacopeia: The National Formulary (USP 24 NF19) published in 1999.]

Dosage forms and formulations may be manufactured by appropriate methods known in the art for obtaining a structure for producing a beneficial pharmacokinetic profile, in particular a sustained pharmacokinetic profile. For example, solid dose oral immediate release dosage forms are marketed by Cima Labs, Fuisz Technologies Ltd., Prographarm, R. P. Scherer, and Yamanouchi-Shaklee. A sustained release dosage form can be made using standard techniques including but not limited to those disclosed in U.S. Pat. No. 5,980,942 to Katzhendler et al; Development of a Controlled Release Matrix Tablet Containing a Water-Soluble Drug Utilizing Hypromellose and Ethylcellulose. Dasbach, T et al., The Dow Chemical Company, Midland, Mich. 48674; The Effect of Process Conditions on Various Sustained Release Formulations During Wet Granulation. Inbasekaran P. and Balwinski, K. The Dow Chemical Company, Midland, Mich. 48674; Direct Compression of Sustained-Release Hydrophilic Matrix Tablets Containing Hypromellose and MCC: Effects of a Lubricant T. D. Cabelka Technical Service and Development for METHOCEL Cellulose Ethers Larkin Laboratory, The Dow Chemical Company, Midland, Mich. 48674 USA; and, Lab-Scale to Full Production Scale Evaluation of a Controlled-Release Formulation Based on Hypromellose and Manufactured Using Roll Compaction Technology Sheskey 1, P. et al., The Dow Chemical Company Larkin Laboratory Midland, Mich. 48674. 2 The Vector Corporation Marion, Iowa 52302.

A dosage form or formulation of the invention typically comprises pharmaceutically acceptable carriers, diluents, or excipients which do not interfere with the effectiveness or activity of the active ingredient and which are not toxic to patients. A carrier, excipient, or vehicle includes without limitation, diluents, binders, adhesives, lubricants, disintegrates, bulking agents, wetting or emulsifying agents, pH buffering agents, and miscellaneous materials such as absorbants that may be needed in order to manufacture or deliver a formulation or dosage form of the invention to provide a beneficial pharamacokinetic profile. Examples of suitable carriers, diluents, or excipients are discussed below.

Diluents useful for the manufacture of dosage forms or formulations of the invention include microcrystalline cellulose (e.g., Avicel FMC Corp., Philadelphia, Pa.), for example grades of microcrystalline cellulose to which binders such as hydroxypropyl methyl cellulose have been added, waxes such as paraffin, modified vegetable oils, carnauba wax, hydrogenated castor oil, beeswax, and the like, as well as polymers such as cellulose, cellulose esters, cellulose ethers, poly(vinyl chloride), poly(vinyl acetate), copolymers of vinyl acetate and ethylene, polystyrene, and the like. In aspects of the invention the mean particle size for the microcrystalline cellulose generally ranges from about 90 μm to about 200 μm. Microcrystalline cellulose may be present in an amount from about 10 wt % to about 70 wt %, in particular in an amount of about 30-70 wt %.

A dosage form or formulation of the invention may optionally comprise water soluble binders or release modifying agents including sugars, salts, water-soluble polymers, for example celluloses such as ethylcellulose, hydroxymethylcellulose, hydroxypropyl cellulose (H PC), hydroxypropyl methyl cellulose HPMC), methyl cellulose, poly (N-vinyl-2-pyrrolidinone) (PVP), poly(ethylene oxide) (PEO), polypropylpyrrolidone, poly(vinyl alcohol) (PVA), polyethylene glycol, starch, natural and synthetic gums (e.g., acacia, alginates, and gum arable) and other such natural and synthetic materials, and waxes. Suitable water-soluble materials include lactose, sucrose, glucose, and mannitol, as well as HPC, HPMC; and PVP.

A dosage form or formulation of the invention in the form of a tablet may optionally comprise lubricants to prevent a tablet or punches from sticking in the die. Examples of lubricants include calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc and zinc stearate. A lubricant is present, for example, in an amount from about 0.25 wt % to about 4.0% wt %.

A dosage form or formulation of the invention may optionally comprise disintegrants to break up the dosage form and release a cyclic polyalcohol compound. Examples of disintegrants include sodium starch glycolate, sodium carboxymethyl cellulose, calcium carboxymethyl cellulose, croscarmellose sodium, polyvinylpyrrolidone, methyl cellulose, microcrystalline cellulose, powdered cellulose, lower alkyl-substituted hydroxypropyl cellulose, polacrilin potassium, starch, pregelatinized starch and sodium alginate. The amount of disintegrant included in a dosage form will depend on factors, including the properties of the dispersion, and the properties of the disintegrant selected. A disintegrant may generally comprise from 1 wt % to 15 wt %, preferably from 1 wt % to 10 wt % of the dosage form.

A dosage form or formulation of the invention may optionally comprise solubilizing acid excipients to increase the release rate of cyclohexane polyalcohol compound, increase the total quantity of cyclohexane polyalcohol compound released, and potentially increase absorption and consequently the bioavailability of cyclohexane polyalcohol compound, particularly from matrix formulations that release cyclohexane polyalcohol compound over a period of six hours or longer. Examples of solubilizing acid excipients include malic acid, citric acid, erythorbic acid, ascorbic acid, adipic acid, glutamic acid, maleic acid, aconitic acid, and aspartic acid and solubilizing excipients such as partial glycerides, glycerides, glyceride derivatives, polyethylene glycol esters, polypropylene glycol esters, polyhydric alcohol esters, polyoxyethylene ethers, sorbitan esters, polyoxyethylene sorbitan esters, saccharide esters, phospholipids, polyethylene oxide-polypropylene oxide block co-polymers, and polyethylene glycols.

A sustained release dosage form or formulation of the invention may optionally comprise reducing carbohydrates. Reducing carbohydrates are generally sugars and their derivatives that contain a free aldehyde or ketone group capable of acting as a reducing agent through the donation of electrons. Suitable reducing carbohydrates include monosaccharides and disaccharides and more specifically include lactose, glucose, fructose, maltose and other similar sugars. A dosage form or formulation may comprise less than about 20% by weight of reducing carbohydrates.

Excipients that may be used in dosage forms and formulations of the invention include starch, mannitol, kaolin, calcium sulfate, inorganic salts (e.g., sodium chloride), powdered cellulose derivatives, tribasic calcium phosphate, calcium sulfate, magnesium carbonate, magnesium oxide, poloxamers such as polyethylene oxide and hydroxypropyl methylcellulose.

A dosage form or formulation of the invention may also comprise polymers which are insoluble in aqueous media and are thermoplastic i.e., polymer-based release-controlling components. Examples of such polymers include cellulose ethers such as cellulose acetate, cellulose propionate, cellulose butyrate, cellulose acetate butyrate, ethylcellulose, hydroxypropylmethylcellulose, etc.

Strategies to achieve sustained release of a cyclohexane polyalcohol compound are known in the art and include without limitation diffusion systems (e.g., reservoir devices and matrix devices), dissolution systems such as encapsulated dissolution systems (e.g, tiny time pills) and matrix dissolution systems, combination diffusion/dissolution systems, osmotic systems, and ion-exchange resin systems as described in in the standard text, Remington: The Science and Practice of Pharmacy (21^{st} Edition. 2005, University of the Sciences in Philadelphia (Editor), Mack Publishing Company).

A class of sustained-release dosage forms include tablets with or without multiparticulates. A tablet can comprise multiparticulates that have been mixed with a binder, disintegrants, or other excipients known in the art, and then formed into a tablet using compressive forces. Suitable binders include microcrystalline cellulose, starch, gelatin, polyvinyl pyrrolidinone, polyethylene glycol, and sugars such as sucrose, glucose, dextrose, and lactose. Suitable disintegrants include sodium starch glycolate, croscarmellose sodium, crospovidone, and sodium carboxymethyl cellulose. In an embodiment, a tablet includes an effervescent agent (acid-base combinations) that generates carbon dioxide after administration to assist in the disintegration of the tablet. Multiparticulates, binder, and other excipients may be granulated prior to formation of the tablet. Well known wet- or dry-granulation processes, direct compression or non-compression processe may be used to produce a multiparticulate tablets.

A sustained release dosage form can be in the form of a capsule including solid dosage forms in which multiparticulates and optionally excipients are enclosed in either a hard or soft, soluble container or shell. A “capsule” also includes dosage forms for which the body of the dosage form remains substantially intact during its residence in the use environment. Upon administration, the shell of the capsule typically dissolves or disintegrates, releasing the contents of the capsule. Capsules may be produced using processes well known in the art.

A sustained release dosage form may also be in the form of pills i.e. small, round solid dosage forms that comprise multiparticulates mixed with a binder and other excipients. Upon administration, the pill disintegrates, allowing the multiparticulates to be dispersed therein. Pills may be produced using processes well-known in the art.

### Multicomponent Dosage Forms

In one embodiment, the present invention provides a multiparticulate modified release composition which delivers a cyclohexane polyalcohol compound in a pulsatile manner providing a plasma profile similar to two sequential doses of an immediate release dosage form.

In still another embodiment, the present invention provides a multiparticulate modified release composition which delivers cyclohexane polyalcohol compound in a continuous manner.

In yet another embodiment, the present invention provides a multiparticulate modified release composition in which a first portion of cyclohexane polyalcohol compound is released immediately upon administration and one or more subsequent portions of cyclohexane polyalcohol compound are released after an initial time delay.

In an embodiment, the present invention provides a multiparticulate modified release composition in which the particles may, as desired, contain a modified release coating and/or a modified release matrix material.

According to one aspect of the present invention, there is provided a pharmaceutical composition having a first component comprising active ingredient-containing particles, and at least one subsequent component comprising active ingredient-containing particles, each subsequent component having a rate and/or duration of release different from the first component wherein at least one of said components comprises particles containing cyclohexane polyalcohol compound. The drug-containing particles may be coated with a modified release coating. Alternatively or additionally, the drug-containing particles may comprise a modified release matrix material. Following oral delivery, the composition delivers cyclohexane polyalcohol compound in a pulsatile manner. In one embodiment, the first component provides an immediate release of cyclohexane polyalcohol compound, and the one or more subsequent components provide a sustained release of cyclohexane polyalcohol compound. In such embodiments, the immediate release component serves to hasten the onset of action by minimizing the time from administration to a therapeutically effective plasma concentration level, and the one or more subsequent components serve to minimize the variation in plasma concentration levels and/or maintain a therapeutically effective plasma concentration throughout the dosing interval.

The modified release coating and/or the modified release matrix material may cause a lag time between the release of the active ingredient from the first population of active ingredient-containing particles and the release of the active ingredient from subsequent populations of active ingredient-containing particles. Where more than one population of active ingredient-containing particles provide a modified release, the modified release coating and/or the modified release matrix material may cause a lag time between the release of the active ingredient from the different populations of active ingredient-containing particles. The duration of these lag times may be varied by altering the composition and/or the amount of the modified release coating and/or altering the composition and/or amount of modified release matrix material utilized. Thus, the duration of the lag time can be designed to mimic a desired plasma profile, such as a twice daily dosing profile from an immediate release formulation.

Because the plasma profile produced by the modified release composition upon administration is substantially similar to the plasma profile produced by the administration of two or more immediate release dosage forms given sequentially, the modified release composition of the present invention is particularly useful for administering a cyclohexane polyalcohol compound. Accordingly in another aspect of the present invention, the composition can be designed to produce a plasma profile that minimizes or eliminates the variations in plasma concentration levels associated with the administration of two or more immediate release dosage forms given sequentially. In such embodiments, the composition may be provided with an immediate release component to hasten the onset of action by minimizing the time from administration to a therapeutically effective plasma concentration level, and at least one modified release component to maintain a sustained release profile with a therapeutically effective plasma concentration level throughout the dosing interval.

The active ingredients in each component may be the same or different. For example, the composition may comprise components comprising only cyclohexane polyalcohol compound as the active ingredient. Alternatively, the composition may comprise a first component comprising cyclohexane polyalcohol compound, and at least one subsequent component comprising an active ingredient other than cyclohexane polyalcohol compound, suitable for co-administration with cyclohexane polyalcohol compound, or a first component containing an active ingredient other than cyclohexane polyalcohol compound, and at least one subsequent component comprising cyclohexane polyalcohol compound. Two or more active ingredients may be incorporated into the same component when the active ingredients are compatible with each other. An active ingredient present in one component of the composition may be accompanied by, for example, an enhancer compound or a sensitizer compound in another component of the composition, in order to modify the bioavailability or therapeutic effect thereof.

As used herein, the term “enhancer” refers to a compound which is capable of enhancing the absorption and/or bioavailability of an active ingredient by promoting net transport across the gastrointestinal tract in an animal, such as a human. Enhancers include but are not limited to: medium chain fatty acids, salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

In those embodiments in which more than one drug-containing component is present, the proportion of cyclohexane polyalcohol compound contained in each component may be the same or different depending on the desired dosing regime. The cyclohexane polyalcohol compound present in the first component and in subsequent components may be any amount sufficient to produce a therapeutically effective plasma concentration level, preferably at a constant level.

The time release characteristics for the delivery of cyclohexane polyalcohol compound from each of the components may be varied by modifying the composition of each component, including modifying any of the excipients and/or coatings which may be present. In particular, the release of cyclohexane polyalcohol compound may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the active ingredient may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time and/or time delay for the release of cyclohexane polyalcohol compound from each component may also be varied by modifying the composition of each of the components, including modifying any excipients and coatings which may be present. For example, the first component may be an immediate release component wherein cyclohexane polyalcohol compound, is released immediately upon administration. The second and subsequent component(s) may be, for example, a time-delayed immediate release component as just described or, alternatively, a time-delayed sustained release or extended release component in which cyclohexane polyalcohol compound, is released in a controlled fashion over an extended period of time.

As will be appreciated by those skilled in the art, the exact nature of the plasma concentration curve will be influenced by the combination of all of these factors just described. In particular, the lag time between the delivery and the onset of absorption of the cyclohexane polyalcohol compound, in each component containing cyclohexane polyalcohol compound may be controlled by varying the composition and coating (if present) of each of the components. Thus by variation of the composition of each component (including the amount and nature of the active ingredient(s)) and by variation of the lag time, numerous release and plasma profiles may be obtained. Depending on the duration of the lag time between the release of cyclohexane polyalcohol compound from each component and the nature of the release of cyclohexane polyalcohol compound from each component (i.e. immediate release, sustained release etc.), the plasma profile may be continuous (i.e., having a single maximum) or pulsatile in which the peaks in the plasma profile may be well separated and clearly defined (e.g. when the lag time is long) or superimposed to a degree (e.g. when the lag time is short), as would be the case for bid dosing schedules of immediate release dosage forms.

The plasma profile produced from the administration of a single dosage unit comprising the composition of the present invention is advantageous when it is desirable to deliver two pulses of active ingredient without the need for the sequential administration of two dosage units.

Any coating material which modifies the release of cyclohexane polyalcohol compound, in the desired manner may be used. In particular, coating materials suitable for use in the practice of the present invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the trademark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers—in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (mol. wt. ˜5 k-5,000 k), polyvinylpyrrolidone (mol. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (mol. wt. ˜30 k-300 k), polysaccharides such as agar, acacia, karaya, tragacanth, algins and guar, polyacrylamides, Polyox® polyethylene oxides (mol. wt. ˜100 k-5,000 k), AquaKeep® acrylate polymers, diesters of polyglucan, crosslinked polyvinyl alcohol and poly N-vinyl-2-pyrrolidone, sodium starch glucolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers with methacrylic acid, other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. As will be appreciated by the person skilled in the art, excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of cyclohexane polyalcohol compound, dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate and mixture thereof.

A modified release composition according to the present invention may be incorporated into any suitable dosage form which facilitates release of the active ingredient in a pulsatile manner. In one embodiment, the dosage form comprises a blend of different populations of active ingredient-containing particles which make up the immediate release and the modified release components, the blend being filled into suitable capsules, such as hard or soft gelatin capsules. Alternatively, the different individual populations of active ingredient-containing particles may be compressed (optionally with additional excipients) into mini-tablets which may be subsequently filled into capsules in the appropriate proportions. Another suitable dosage form is that of a multilayer tablet. In this instance the first component of the modified release composition may be compressed into one layer, with the subsequent component being subsequently added as a subsequent layer of the multilayer tablet. The populations of the particles making up the composition of the invention may further be included in rapidly dissolving dosage forms such as an effervescent dosage form or a fast-melt dosage form.

In one embodiment, the composition comprises at least two components containing cyclohexane polyalcohol compound: a first component and one or more subsequent components. In such embodiment, the first component of the composition may exhibit a variety of release profiles including profiles in which substantially all of the cyclohexane polyalcohol compound contained in the first component is released rapidly upon administration of the dosage form, released rapidly but after a time delay (delayed release), or released slowly over time. In one such embodiment, the cyclohexane polyalcohol compound contained in the first component is released rapidly upon administration to a patient. As used herein, “released rapidly” includes release profiles in which at least about 20%-60% of the active ingredient of a component is released within about an hour after administration, the term “delayed release” includes release profiles in which the active ingredient of a component is released (rapidly or slowly) after a time delay, and the terms “controlled release” and “extended release” include release profiles in which at least about 40%-80% of the active ingredient contained in a component is released slowly.

The second component of such embodiment may also exhibit a variety of release profiles including an immediate release profile, a delayed release profile or a controlled release profile. In one such embodiment, the second component exhibits a delayed release profile in which cyclohexane polyalcohol compound is released after a time delay.

The plasma profile produced by the administration of dosage forms of the present invention which comprise an immediate release component comprising cyclohexane polyalcohol compound, or microparticles containing cyclohexane polyalcohol compound, and at least one modified release component comprising cyclohexane polyalcohol compound, or microparticles containing cyclohexane polyalcohol compound, can be substantially similar to the plasma profile produced by the administration of two or more IR dosage forms given sequentially, or to the plasma profile produced by the administration of separate IR and modified release dosage forms. Accordingly, the dosage forms of the present invention can be particularly useful for administering cyclohexane polyalcohol compound, where the maintenance of pharmacokinetic parameters may be desired but are complex.

In one embodiment, the composition and the solid oral dosage forms containing the composition release cyclohexane polyalcohol compound, such that substantially all of the cyclohexane polyalcohol compound contained in the first component is released prior to release of cyclohexane polyalcohol compound from the at least one subsequent component. When the first component comprises an IR component, for example, it is preferable that release of the cyclohexane polyalcohol compound from the at least one subsequent component is delayed until substantially all cyclohexane polyalcohol compound in the IR component has been released. Release of cyclohexane polyalcohol compound from the at least one subsequent component may be delayed as detailed above by the use of a modified release coatings and/or a modified release matrix material.

As described herein, the present invention also includes various types of modified release systems by which cyclohexane polyalcohol compound, may be delivered in either a pulsatile or continuous manner. These systems include but are not limited to: films with cyclohexane polyalcohol compound, or microparticles containing cyclohexane polyalcohol compound, in a polymer matrix (monolithic devices); systems in which cyclohexane polyalcohol compound, or microparticles containing the same, is contained by a polymer (reservoir devices); polymeric colloidal particles or microencapsulates (microparticles, microspheres or nanoparticles) in the form of reservoir and matrix devices; systems in which cyclohexane polyalcohol compound, or microparticles containing the same, is contained by a polymer which contains a hydrophilic and/or leachable additive e.g., a second polymer, surfactant or plasticizer, etc. to give a porous device, or a device in which cyclohexane polyalcohol compound release may be osmotically controlled (both reservoir and matrix devices); enteric coatings (ionizable and dissolve at a suitable pH); (soluble) polymers with (covalently) attached pendent cyclohexane polyalcohol compound molecules and devices where release rate is controlled dynamically: e.g., the osmotic pump.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Röhm and Haas) range of poly(acrylate, methacrylate) copolymers are known in the art.

### Monolithic Devices (Matrix Devices)

Monolithic (matrix) devices may be used for controlling the release of a drug. This is possible because they are relatively easy to fabricate compared to reservoir devices, and the danger of an accidental high dosage that could result from the rupture of the membrane of a reservoir device is not present. In such a device, the active agent is present as a dispersion within the polymer matrix, and they are typically formed by the compression of a polymer/drug mixture or by dissolution or melting. The dosage release properties of monolithic devices may be dependent upon the solubility of the drug in the polymer matrix or, in the case of porous matrixes, the solubility in the sink solution within the particle's pore network, and also the tortuosity of the network (to a greater extent than the permeability of the film), dependent on whether the drug is dispersed in the polymer or dissolved in the polymer. For low loadings of drug (0 to 5% W/V), the drug will be released by a solution-diffusion mechanism (in the absence of pores). At higher loadings (5 to 10% W/V), the release mechanism will be complicated by the presence of cavities formed near the surface of the device as the drug is lost: such cavities fill with fluid from the environment increasing the rate of release of the drug.

It is common to add a plasticizer (e.g., a poly(ethylene glycol), abbreviated as PEG), a surfactant, or adjuvant (i.e., an ingredient which increases effectiveness), to matrix devices (and reservoir devices) as a means to enhance the permeability (although, in contrast, plasticizers may be fugitive, and simply serve to aid film formation and, hence, decrease permeability.).

Surfactants on (hydrophobic) matrix devices may increase the release rate of a drug by three possible mechanisms: (i) increased solubilization, (ii) improved ‘wettability’ to the dissolution media, and (iii) pore formation as a result of surfactant leaching. Examples of suitable surfactants include Eudragit brand surfactants such as Eudragit® RL 100, Eudragit® RS, Eudragit® RL, and RS 100 plasticized by sorbitol. The greatest influence on release is effected by surfactants that are more soluble due to the formation of disruptions in the matrix allowing the dissolution medium access to within the matrix. Composite devices consisting of a polymer/drug matrix coated in a polymer containing no drug also exist. Such a device may be formed from aqueous Eudragit® lattices, and provides a continuous release by diffusion of the drug from the core through the shell. Similarly, a polymer core containing the drug may be produced and coated with a shell that is eroded by gastric fluid. The rate of release of drug compound from this shell may be relatively linear (a function of the rate limiting diffusion process through the shell) and inversely proportional to the shell thickness, whereas the release from a core as described alone may decrease with time.

A sustained release dosage form contemplated by the present invention includes matrix systems, in which a cyclohexane polyalcohol compound is dissolved, embedded or dispersed in a matrix of another material that serves to slow the release of the cyclohexane polyalcohol compound in vivo. A matrix system may be a matrix tablet that remains substantially intact during the period of sustained release. Matrix tablets may be partially coated with a polymer which impedes the release of cyclohexane polyalcohol compound. Matrix materials useful for the manufacture of dosage forms include diluents such as microcrystalline cellulose (e.g., AviceltE FMC Corp., Philadelphia, Pa.). A sustained release dosage form may be a non-eroding matrix-system comprising a cyclohexane polyalcohol compound dispersed in a hydrogel matrix. Examples of materials for forming hydrogels include hydrophilic vinyl and acrylic polymers, polysaccharides such as calcium alginate, and poly(ethylene oxide), in particular poly(2-hydroxyethyl methacrylate), poly(acrylic acid), poly(methacrylic acid, poly(N-vinyl-2-pyrrolidinone), poly(vinyl alcohol) and their copolymers with each other and with hydrophobic monomers such as methyl methacrylate, vinyl acetate, and the like; and hydrophilic polyurethanes containing large poly(ethylene oxide) blocks. A hydrogel may comprise interpenetrating networks of polymers, which may be formed by addition or by condensation polymerization. Matrix tablets can be made by tabletting methods common in the art.

A matrix system may contain multiparticulates comprising a plurality of cyclohexane polyalcohol compound-containing particles, each particle comprising a mixture of cyclohexane polyalcohol compound with one or more excipients selected to form a matrix capable of limiting the dissolution rate of the cyclohexane polyalcohol compound into an aqueous medium. Suitable matrix materials include water-insoluble materials such as waxes, cellulose, or other water-insoluble polymers, in particular microcrystalline cellulose. A matrix system may also comprise water soluble release modifying agents, release modifying agents, solubilizing acids or surfactant type excipients and the like. Matrix multiparticulates can be produced using methods in the art including without limitation extrusion/spheronization processes or rotary granulation processes, or by coating the compounds, matrix-forming excipients and other matrix materials onto seed cores; or forming wax granules. Once formed, cyclohexane polyalcohol compound matrix multiparticulates can be blended with compressible excipients such as lactose, microcrystalline cellulose, dicalcium phosphate, and the like and the blend compressed to form a tablet. Disintegrants may also be employed in matrix systems. Tablets prepared by this method disintegrate when placed in an aqueous medium, thereby exposing the multiparticulates which release the cyclohexane polyalcohol compound. Cyclohexane polyalcohol compound matrix multiparticulates can also be filled into capsules, such as hard gelatin capsules. In an embodiment, a hydrophilic matrix tablet is provided that releases a cyclohexane polyalcohol compound from the matrix by diffusion, erosion or dissolution of the matrix, or a combination of these mechanisms, and optionally comprises multiparticulates.

A matrix system dosage form may be coated or partially coated to improve the release rate of the cyclohexane polyalcohol compound. In an aspect, a matrix tablet is coated with an impermeable coating, and a hole or opening is provided by which the content of the tablet is exposed. (See for example, U.S. Pat. No. 4,792,448 to Ranade, and Hansson et al., J. Pharm. Sci. 77 (1988) 322-324) Examples of coating materials include film-forming polymers and waxes, in particular thermoplastic polymers, such as poly(ethylene-co-vinyl acetate), poly(vinyl chloride), ethylcellulose, and cellulose acetate.

### Enteric Films

Enteric coatings consist of pH sensitive polymers as described in the art. Typically the polymers are carboxylated and interact very little with water at low pH, while at high pH the polymers ionize causing swelling or dissolution of the polymer. Coatings can therefore be designed to remain intact in the acidic environment of the stomach, protecting either the drug from this environment or the stomach from the drug, but to dissolve in the more alkaline environment of the intestine. The core of the tablet or dosage form may be adapted to sustained release so that the release rate of the drug is maintained over time.

### Reservoir Devices

Cyclohexane polyalcohol compound sustained-release dosage forms of the invention may include membrane-moderated or reservoir systems. A typical approach to modified release is to encapsulate or contain the drug entirely (e.g., as a core), within a polymer film or coat (i.e., microcapsules or spray/pan coated cores). Various techniques can affect the diffusion process may readily be applied to reservoir devices (e.g., the effects of additives, polymer functionality (and, hence, sink-solution pH) porosity, film casting conditions, etc.) and, hence, the choice of polymer must be an important consideration in the development of reservoir devices. Modeling the release characteristics of reservoir devices (and monolithic devices) in which the transport of cyclohexanehexyl, is by a solution-diffusion mechanism for the relevant boundary conditions. When the active agent is in a saturated suspension, the driving force for release is kept constant until the device is no longer saturated providing a substantially continuous release profile. Alternatively the release-rate kinetics may be desorption controlled, and a function of the square root of time, another modified release. Examples of reservoir dosage forms include membrane-coated diffusion based capsules, tablets, or systems comprising multiparticulates. In this dosage form, a reservoir of cyclohexane polyalcohol compound is surrounded by a rate-limiting membrane. The cyclohexane polyalcohol compound crosses the membrane by mass transport mechanisms, for example, a mechanism involving dissolution in the membrane followed by diffusion across the membrane or diffusion through liquid-filled pores within the membrane. An individual reservoir system dosage form can be large, such as a tablet containing a single large reservoir, or a system comprising a multiparticulate, such as a capsule containing a plurality of reservoir particles, each individually coated with a membrane. A coating for use in a reservoir system can be non-porous and permeable to a cyclohexane polyalcohol compound (for example, a cyclohexane polyalcohol compound may diffuse directly through the membrane), or it can be porous. The membrane can be prepared from sustained release coatings known in the art, such as a cellulose ester or ether, an acrylic polymer, Eudragit brand polymers, such as Eudragit RS100® or a mixture of polymers.

In an aspect of the invention, the reservoir systems are tablets. Tablet cores containing cyclohexane polyalcohol compound can be made by a variety of techniques standard in the pharmaceutical industry. Cores can be coated with a rate-controlling coating which allows the cyclohexane polyalcohol compound in the reservoir, or tablet core, to diffuse out through the coating at the desired rate.

Another example of a reservoir system is a dosage form comprising a multiparticulate wherein each particle is coated with a polymer designed to provide sustained release of a cyclohexane polyalcohol compound. Each multiparticulate particle comprises a cyclohexane polyalcohol compound and one or more excipients as required for fabrication and performance. A sustained release coating known in the art, in particular polymer coatings, can be employed to prepare the membrane. A membrane coating can also be modified by the addition of plasticizers known in the art.

### Osmotically Controlled Devices

Cyclohexane polyalcohol compound sustained-release dosage forms include osmotic delivery devices or “osmotic pumps”. Osmotic pumps comprise a core containing an osmotically effective composition surrounded by a semipermeable membrane. Water passes through the membrane but solutes dissolved in water permeate through the membrane at a rate significantly slower than water. When placed in an aqueous environment, the device takes in water due to the osmotic activity of the core composition. As a result of the semipermeable nature of the surrounding membrane, the contents of the device (including a cylcohexane polyalcohol and any excipients) cannot pass through the non-porous regions of the membrane and are driven by osmotic pressure to leave the device through an opening or passageway in the dosage form. The passageway can be incorporated in the device in the manufacturing process, formed in situ by the rupture of intentionally-incorporated weak points in the coating under the influence of osmotic pressure, or formed in situ by dissolution and removal of water soluble porosigens incorporated in the coating. An osmotically effective composition generally includes water-soluble species, which generate a colloidal osmotic pressure, and water swellable polymers. Examples of materials useful for forming a semipermeable membrane include polyamides, polyesters, and cellulose derivatives, preferably cellulose ethers and esters. Preferred materials are those which spontaneously form one or more exit passageways, either during manufacturing or when placed in an environment of use, including polymers with pores formed by phase inversion during manufacturing or by dissolution of a water-soluble component present in the membrane. An osmotic delivery device can comprise a coated bi-layer tablet, cyclohexane polyalcohol compound multiparticulates coated with an asymmetric membrane, or osmotic capsules. The release rate remains substantially constant as a function of the influx of the aqueous surrounding environment, delivering a volume approximately equal to the volume of solvent uptake.

### Coated Swellable Tablets

Another cyclohexane polyalcohol compound sustained release dosage form is a coated swellable tablet described in EP 378404A2 or U.S. Pat. No. 5,792,471. The tablets comprise a tablet core comprising a cyclohexane polyalcohol compound and a swelling material, (e.g., a hydrophilic polymer), coated with a membrane which contains holes or pores through which swelling material can extrude and carry out the cyclohexane polyalcohol compound. Alternatively, the membrane can comprise polymeric or low molecular weight water soluble porosigens which dissolve in an aqueous environment, providing pores through which the swelling material and cyclohexane polyalcohol compound can extrude. Suitable porosigens include low molecular weight compounds like glycerol, sucrose, glucose, and sodium chloride and water-soluble polymers such as hydroxypropylmethylcellulose (HPMC). Holes or pores can be formed in the coating by drilling holes in the coating using a laser or other mechanical means. The membrane material can comprise any film-forming polymer, including polymers which are water permeable or impermeable, providing that the membrane deposited on the tablet core is porous or contains water-soluble porosigens or possesses a macroscopic hole for exit of water and cyclohexane polyalcohol compound release. Also contemplated are multiparticulates (or beads) with a cyclohexane polyalcohol compound/swellable material core, coated by a porous or porosigen-containing membrane. A coated swellable tablet dosage form can also be multilayered, as described in EP 378404A2 or U.S. Pat. No. 5,792,471.

The invention also contemplates combination dosage forms comprising a combination of sustained release characteristics and immediate release characteristics. For example, a formulation or dosage form of the invention may be in the form of an oral tablet which includes an immediate release portion comprising a cyclohexane polyalcohol compound, providing for a rapid onset of therapeutic effect, and a sustained release portion of a cyclohexane polyalcohol compound, providing for a relatively longer duration of therapeutic effect. (Combination dosage forms are also described for example, in US Published Application No. 2003009272 and U.S. Pat. No. 6,908,626.)

### Applications

The invention contemplates the use of a formulation or dosage form of the invention for treating a disorder and/or disease, in particular preventing, and/or ameliorating disease severity, disease symptoms, and/or periodicity of recurrence of a disorder and/or disease disclosed herein. The invention also contemplates preventing and/or treating in mammals, disorders and/or diseases using formulations, dosage forms or treatments of the invention.

In an aspect, the invention provides a method of improving memory of a healthy subject or the memory of a subject with age impaired memory by administering an effective amount of a formulation or dosage form of the invention.

In another aspect, the present invention relates to a method for improving memory, especially short-term memory and other mental dysfunction associated with the aging process comprising administering an effective amount of a formulation or dosage form of the invention.

In an embodiment, a method is provided for treating a mammal in need of improved memory, wherein the mammal has no diagnosed disease, disorder, infirmity or ailment known to impair or otherwise diminish memory, comprising the step of administering to the mammal an effective memory-improving amount of a formulation or dosage form of the invention.

In another aspect of the invention, a method is provided for treating in a subject a condition of the central or peripheral nervous system or systemic organ associated with a disorder in protein folding or aggregation, or amyloid formation, deposition, accumulation, or persistence, comprising administering to the subject a therapeutically effective amount of a formulation or dosage form of the invention.

In a further aspect, the invention provides a method involving administering to a subject a formulation or dosage form of the invention which inhibits amyloid formation, deposition, accumulation and/or persistence, and/or which causes dissolution/disruption of pre-existing amyloid. Thus, formulations and dosage forms of the invention may be used for inhibiting amyloidosis in disorders in which amyloid deposition occurs.

In another aspect, the invention provides a method for treating in a subject a condition associated with an amyloid interaction that can be disrupted or dissociated with a cyclohexane polyalcohol compound comprising administering to the subject a therapeutically effective amount of a formulation or dosage form of the invention.

In an aspect, the invention provides a method for preventing, reversing, reducing or inhibiting amyloid protein assembly, enhancing clearance of amyloid deposits, or slowing deposition of amyloid deposits in a subject comprising administering a formulation or dosage form of the invention.

In an aspect, the invention provides a method for preventing, reversing, reducing or inhibiting amyloid fibril formation, organ specific dysfunction (e.g., neurodegeneration), or cellular toxicity in a subject comprising administering to the subject a therapeutically effective amount a formulation or dosage form of the invention.

In another aspect, the invention provides a method of preventing or reversing conformationally altered protein assembly or aggregation in an animal that includes introducing a formulation or dosage form of the invention to the conformationally altered protein.

In a still further aspect of the invention, a method of treating conformationally altered protein assembly or aggregation in an animal is provided that includes administering a therapeutically effective amount of a formulation or dosage form of the invention.

In an aspect, the invention provides a method for increasing or maintaining synaptic function in a subject comprising administering a therapeutically effective amount of a formulation or dosage form of the invention.

The invention has particular applications in treating a disorder and/or disease characterized by amyloid deposition, in particular an amyloidoses, more particularly Alzheimer's disease. Thus, the invention relates to a method of treatment comprising administering a therapeutically effective amount of a formulation or dosage form of the invention, which upon administration to a subject with symptoms of a disease characterized by amyloid deposition, more particularly Alzheimer's disease, produces beneficial pharmacokinetic profiles, in particular sustained pharmacokinetic profiles. In an embodiment, the treatment is evidenced by one or more of the following: disruption of aggregated Aβ or Aβ oligomers; increased or restored long term potentiation; maintenance of or increased synaptic function; reduced cerebral accumulation of Aβ, reduced deposition of cerebral amyloid plaques; reduced soluble Aβ oligomers in the brain; reduced glial activity; reduced inflammation; and/or, reduced cognitive decline or improvement of cognitive abilities.

In an aspect, the invention provides a method for amelioriating progression of a disorder and/or disease or obtaining a less severe stage of a disease in a subject suffering from such disease (e.g. Alzheimer's disease) comprising administering a therapeutically effective amount of a formulation or dosage form of the invention.

In another aspect, the invention relates to a method of delaying the progression of a disorder and/or disease (e.g. Alzheimer's disease) comprising administering a therapeutically effective amount of a formulation or dosage form of the invention.

In a further aspect, the invention relates to a method of increasing survival of a subject suffering from a disorder and/or disease comprising administering a therapeutically effective amount of a formulation or dosage form of the invention.

In an embodiment, the invention relates to a method of improving the lifespan of a subject suffering from a disorder and/or disease (e.g., Alzheimer's disease) comprising administering a therapeutically effective amount of a formulation or dosage form of the invention.

In an aspect the invention provides a method for treating mild cognitive impairment (MCI) comprising administering a therapeutically effective amount of a formulation or dosage form of the invention.

In an embodiment, the invention provides a method of reducing or reversing amyloid deposition and neuropathology after the onset of cognitive deficits and amyloid plaque neuropathology in a subject comprising administering to the subject a therapeutically effective amount of a formulation or dosage form of the invention.

In another embodiment, the invention provides a method of reducing or reversing amyloid deposition and neuropathology after the onset of cognitive deficits and amyloid plaque neuropathology in a subject comprising administering to the subject an amount of a formulation or dosage form of the invention effective to reduce or reverse amyloid deposition and neuropathology after the onset of cognitive deficits and amyloid plaque neuropathology.

In an aspect, the invention relates to a method for treating Alzheimer's disease comprising contacting Aβ, Aβ aggregates, or Aβ oligomers in particular Aβ40 or Aβ40 aggregates or oligomers and/or Aβ42 or Aβ42 aggregates or oligomers, in a subject with a therapeutically effective amount of a formulation or dosage form of the invention.

In another aspect, the invention provides a method for treating Alzheimer's disease by providing a formulation or dosage form of the invention comprising a cylcohexane polyalcohol compound in an amount sufficient to produce a beneficial pharamacokinetic profile thereby disrupting aggregated Aβ or Aβ oligomers for a prolonged period following administration.

In a further aspect, the invention provides a method for treating Alzheimer's disease in a patient in need thereof which includes administering to the individual a formulation or dosage form of the invention in a form and amount sufficient to produce a beneficial pharamacokinetic profile resulting in increased or restored long term potentiation and/or maintained synaptic function. In another aspect, the invention provides a method for treating Alzheimer's disease comprising administering, preferably orally or systemically, a formulation or dosage form of the invention, to produce a beneficial pharamacokinetic profile thereby reducing one or more of cerebral accumulation of Aβ, deposition of cerebral amyloid plaques, soluble Aβ oligomers in the brain, glial activity, and/or inflammation, for a prolonged period following administration.

The invention in an embodiment provides a method for treating Alzheimer's disease, the method comprising administering to a mammal in need thereof a formulation or dosage form of the invention in an amount sufficient to produce a beneficial pharamacokinetic profile thereby reducing cognitive decline, especially for a prolonged period following administration, to treat the Alzheimer's disease.

The invention in an embodiment provides a method for treating Alzheimer's disease, the method comprising administering to a mammal in need thereof a composition comprising a formulation or dosage form of the invention in an amount sufficient to produce a beneficial pharamacokinetic profile thereby increasing or maintaining synaptic function, especially for a prolonged period following administration, to treat the Alzheimer's disease.

In another aspect, the invention provides a method for preventing and/or treating Alzheimer's disease, the method comprising administering to a mammal in need thereof a composition comprising a formulation or dosage form of the invention in an amount sufficient to disrupt aggregated Aβ or Aβ oligomers for a prolonged period following administration; and determining the amount of aggregated Aβ or Aβ oligomers, thereby treating the Alzheimer's disease. The amount of aggregated Aβ or Aβ oligomers may be measured using an antibody specific for Aβ or a scyllo-inositol labeled with a detectable substance.

In an embodiment, this invention provides a method for treating a disease or disorder disclosed herein in particular a disorder in protein folding and/or aggregation, and/or amyloid formation, deposition, accumulation, or persistence, comprising orally administering to a mammal in need of such treatment, including a human patient, a therapeutically effective amount of cyclohexane polyalcohol compound in a sustained-release dosage form comprising cyclohexane polyalcohol compound or a pharmaceutically acceptable salt thereof, such as an oral dosage form which releases the cyclohexane polyalcohol compound according to a release rate described herein, such as, for example, from about 0.01 mgA/hr to about 50 mgA/hr in a use environment as described herein, such as plasma, brain or CSF

In a further embodiment, this invention provides a method for treating Alzheimer's disease, comprising orally administering to a mammal in need of such treatment, including a human patient, a therapeutically effective amount of cyclohexane polyalcohol compound in a sustained release dosage form comprising cyclohexane polyalcohol compound or a pharmaceutically acceptable salt thereof, such as an oral dosage form which releases the cyclohexane polyalcohol compound according to a release rate described herein, such as, for example, from about 0.01 mgA/hr to about 5 mgA/hr in a use environment as described herein, such as plasma, brain or CSF.

Typically, a preferred range of dosages in the methods of the invention is about 1 mgA to 5 mgA of cyclohexane polyalcohol compound per day and can be as high as about 30 to 35 mgA of cyclohexane polyalcohol compound per day for average adult subjects having a body weight of about 70 kg. For example, the dosage may range from about 1 μg/kg/day to 40 μg/kg/day or within other ranges comprised between 3 μg/kg/day and 30 μg/kg/day.

The invention relates to a method for treating and/or preventing a disorder and/or disease disclosed herein comprising administering a dosage form comprising a cyclohexane polyalcohol compound at a first time point and a second time point in a dosing period, wherein the dose and/or interval between the first and second time point are sufficient to provide a beneficial pharmacokinetic profile whereby the concentration or peak concentration of compound in plasma, brain or CSF does not significantly vary during the dosing period. In an aspect, the dosing period is about 18, 20 or 24 hours. In embodiments of this aspect, the second time point is about 4 to 14 hours, in particular 6 to 14, 6 to 12, or 8 to 12 hours following the first time point. In another aspect, the administration of the compound at the second time point results in concentrations or peak concentrations of the compound in plasma, brain or CSF that do not vary by more than 30%, 20%, 15%, 20%, 5%, or 3% from the concentration or peak concentration of the compound in plasma, brain or CSF following the first time point. In an aspect, the beneficial pharmacokinetic profile is a zero order release profile which does not vary by more than about 20%, 10%, or 5% from the first time point to the second time point of administration. In an aspect, the zero order release profile does not vary by more than about 20%, 10%, or 5% from the first time point to a third time point which is at least 2, 4, 6, 8, 10, 12, 14 or 16 hours following the second time point. In other aspects, the compound is a scyllo-cylcohexanehexyl compound. In embodiments, the dose of the compound is between or from about 1 to 50 mg/kg, 1 to 40 mg/kg, 2.5 to 40 mg/kg, 3 to 40 mg/kg, 3 to 35 mg/kg, most preferably 3 to 30 mg/kg.

The invention relates to a method for treating and/or preventing a disorder and/or disease disclosed herein comprising administering a dosage form comprising a cyclohexane polyalcohol compound at a first time point and a second time point in a dosing period, wherein the dose and/or interval between the first and second time point are sufficient to provide a Cₘᵢₙ in plasma, brain or CSF after the second time point greater than the Cₘᵢₙ after the first time point. In an aspect, the Cₘᵢₙ after the second time point is 5%, 10%, 15%, 20%, 25%, 30%, 35%, 40%, 45%, or 50% greater than the Cₘᵢₙ after the first time point. In an aspect, the dosing period is about 18, 20 or 24 hours. In embodiments of this aspect, the second time point is about 4 to 14 hours, in particular 6 to 14, 6 to 12, or 8 to 12 hours following the first time point. In particular aspects, the dose of the compound is between or from about 1 to 50 mg/kg, 1 to 40 mg/kg, 2.5 to 40 mg/kg, 3 to 40 mg/kg, 3 to 35 mg/kg, most preferably 3 to 30 mg/kg.

The invention relates to a method for treating and/or preventing a disorder and/or disease disclosed herein comprising administering a dosage form comprising a cyclohexane polyalcohol compound at a first time point and a second time point in a dosing period, wherein the dose and/or interval between the first and second time point are sufficient to maintain a concentration of compound in the subject so that Cₘᵢₙ in plasma, brain or CSF after the second time point is greater than the Cₘᵢₙ after the first time point. In an aspect, the Cₘᵢₙ after the second time point is 5%, 10%, 15%, 20%, 25%, 30%, 35%, 40%, 45%, or 50% greater than the Cₘᵢₙ after the first time point. In an aspect, the dosing period is about 18, 20 or 24 hours. In embodiments of this aspect, the second time point is about 4 to 14 hours, in particular 6 to 14, 6 to 12, or 8 to 12 hours following the first time point. In particular aspects, the dose of the compound is between or from about 1 to 50 mg/kg, 1 to 40 mg/kg, 2.5 to 40 mg/kg, 3 to 40 mg/kg, 3 to 35 mg/kg, most preferably 3 to 30 mg/kg.

The present invention also includes the use of formulations, dosage forms and methods in combination treatments with one or more additional therapeutic agents including without limitation beta-secretase inhibitors, gamma-secretase inhibitors, epsilon-secretase inhibitors, other inhibitors of beta-sheet aggregation/fibrillogenesis/ADDL formation (e.g. Alzhemed), NMDA antagonists (e.g. memantine), non-steroidal anti-inflammatory compounds (e.g. Ibuprofen, Celebrex), anti-oxidants (e.g. Vitamin E), hormones (e.g. estrogens), nutrients and food supplements (e.g. Gingko biloba), statins and other cholesterol lowering drugs (e.g. Lovastatin and Simvastatin), acetylcholinesterase inhibitors (e.g. donezepil), muscarinic agonists (e.g. AF102B (Cevimeline, EVOXAC), AF150(S), and AF267B), anti-psychotics (e.g. haloperidol, clozapine, olanzapine), anti-depressants including tricyclics and serotonin reuptake inhibitors (e.g. Sertraline and Citalopram Hbr), statins and other cholesterol lowering drugs (e.g. Lovastatin and Simvastatin), immunotherapeutics and antibodies to Aβ (e.g. ELAN AN-1792), vaccines, inhibitors of kinases (CDK5, GSK3α, GSK3α) that phosphorylate TAU protein (e.g. Lithium chloride), inhibitors of kinases that modulate Aβ production (GSK3α, GSK3α, Rho/ROCK kinases) (e.g. lithium Chloride and Ibuprofen), drugs that upregulate neprilysin (an enzyme which degrades Aβ); drugs that upregulate insulin degrading enzyme (an enzyme which degrades Aβ), agents that are used for the treatment of complications resulting from or associated with a disease, or general medications that treat or prevent side effects. The present invention also includes methods of using the formulations and dosage forms of the invention in combination treatments with one or more additional treatments including without limitation gene therapy and/or drug based approaches to upregulate neprilysin (an enzyme which degrades Aβ), gene therapy and/or drug based approaches to upregulate insulin degrading enzyme (an enzyme which degrades Aβ), or stem cell and other cell-based therapies. Combination treatments may be administered simultaneously and/or sequentially.

Combinations of a formulation or dosage form of the invention and an additional therapeutic agent or treatment may be selected to provide unexpectedly additive effects or greater than additive effects i.e. synergistic effects. Other therapeutics and therapies may act via a different mechanism and may have additive/synergistic effects with the present invention

In an aspect, the invention contemplates the use of a cylcohexane polyalcohol compound for the preparation of a medicament having a beneficial pharmacokinetic profile, in particular sustained pharmacokinetic profile, in treating a disorder and/or disease. The invention additionally provides uses of a formulation or dosage forms of the invention in the preparation of medicaments for the prevention and/or treatment of disorders and/or diseases.

In an embodiment the invention provides the use of a formulation or dosage form of the invention for the preparation of a medicament for prolonged or sustained treatment of Alzheimer's disease.

In a further embodiment the invention provides the use of a formulation or dosage form of the invention for preparation of a medicament to be employed through oral administration for treatment of a disorder characterized by abnormal protein folding and/or aggregation, and/or amyloid formation, deposition, accumulation, or persistence.

Therapeutic efficacy and toxicity of formulations and dosage forms of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Formulations and dosage forms which exhibit large therapeutic indices are preferred. One or more of the therapeutic effects, in particular sustained therapeutic effects disclosed herein, can be demonstrated in a subject or disease model. For example, therapeutic effects may be demonstrated in a model described in the Examples herein, in particular therapeutic effects may be demonstrated in a TgCRND8 mouse with symptoms of Alzheimer's disease.

A formulation or dosage form of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Preclinical Pharmacokinetics and Metabolism

Three studies were performed to investigate the pharmacokinetics of oral administration of a scyllo-cyclohexanehexyl (Table 1):

### Single Dose Pharmacokinetics—Rat

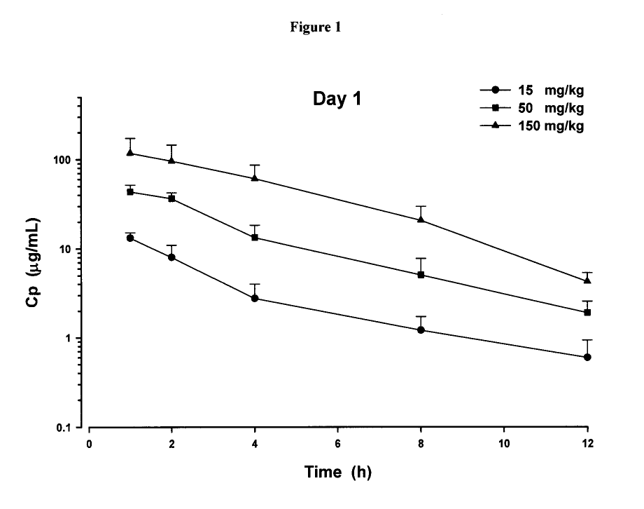

Plasma pharmacokinetics (PK) of a scyllo-cyclohexanehexyl in rats was assessed after single oral doses of 15, 50 and 150 mg/kg. Plots of the mean plasma concentrations versus time are presented in FIG. 1. Derived PK parameters are summarised in Table 2. After single oral doses in rats, maximum plasma concentration (Cₘₐₓ) and exposure (AUC₀₋ₜ) values increased proportionally with dose level of the scyllo-cyclohexanehexyl. Other parameters were unchanged with dose level. The scyllo-cyclohexanehexyl was absorbed rapidly, with mean tₘₐₓ observed between 1.0 and 2.2 hours post-dose. Scyllo-cyclohexanehexyl was cleared relatively rapidly with estimates of half life (t_{1/2}) ranging from 2.1-2.7 hours. Estimates of mean residence time (MRT) were consistent. Volumes of distribution (Vdss) were large, indicating wide distribution of scyllo-cyclohexanehexyl in rats.

### Dog

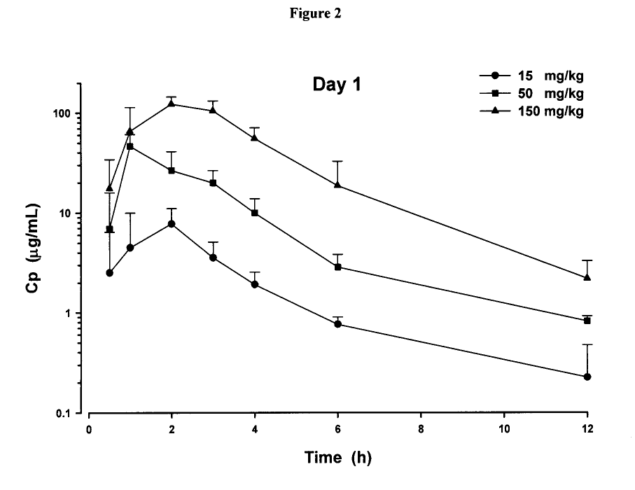

Two studies have investigated the single dose PK of oral administration of a scyllo-cyclohexanehexyl in dogs. The 14 day study investigated 18 animals. Plots of the mean plasma concentrations versus time are presented in FIG. 2. Derived PK parameters are summarised in Table 3. On Day 1 of this study Cₘₐₓ and AUC values increased proportionally with dose level of a scyllo-cyclohexanehexyl. However, dose-normalised exposures showed a trend towards an increase with dose. The scyllo-cyclohexanehexyl was absorbed and cleared rapidly, with tₘₐₓ observed between 1.2 and 2.2 hours post-dose and estimates of t½ ranging from 1.7-2.2 hours. Dose level did not influence tₘₐₓ or t½. At the low dose level (15 mg/kg) Vdss and whole body clearance (Cl/F) were higher than seen at higher doses, emphasizing the rapid distribution of scyllo-cyclohexanhexyl. The extensive brain penetrance of the scyllo-cyclohexanhexyl (see below) may be relevant in this regard.

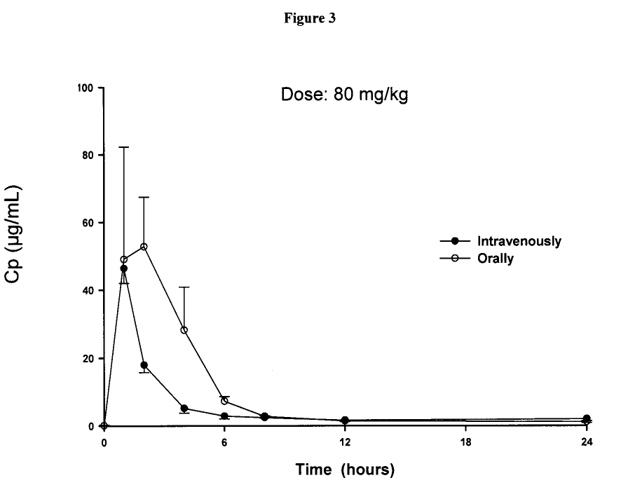

PK of single oral doses of the scyllo-cyclohexanhexyl was also investigated in a preliminary fashion in a dose range-finding study in 5 beagle dogs. Escalating doses were administered in a cross-over design (20, 80 and 240 mg/kg p.o., 80 and 240 mg/kg i.v.). Derived PK parameters are summarised in Table 4. Again AZD-103 was absorbed rapidly, with tₘₐₓ observed between 1.5 and 2 hours following an oral dose. The Cₘₐₓ and AUC values increased proportionally with dose, consistent with observations in the larger study. Estimates of t½ ranged from 2 to 5 hours. Neither dose nor route of administration had any clear effect on t½, MRT, Cl/F or Vdss. The AUC values for the two administration routes suggested a very extensive oral absorption with negligible first-pass effect. At the 80 mg/kg dose level, oral bioavailability was estimated to be >90% (FIG. 3).

### Repeat Dose Pharmacokinetics

### Rat

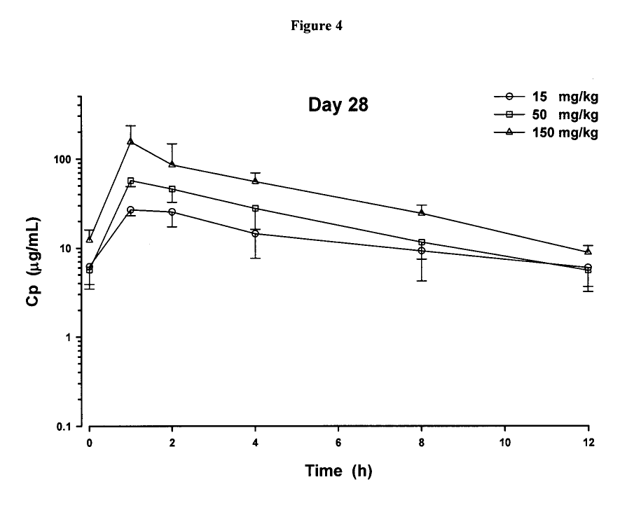

Plasma PK of a scyllo-cyclohexanehexyl compound in rats was assessed after 28 days oral dosing of 15, 50 and 150 mg/kg, administered twice daily. Plots of the mean plasma concentrations versus time are presented in FIG. 4. Derived PK parameters are summarised in Table 5. After 28 days of dosing, Cₘₐₓ and AUC0-t values increased proportionally between 50 and 150 mg/kg dose, as observed after single dose. However, the low dose level (15 mg/kg) after 28 days of dosing showed a marked increase in Cₘₐₓ and AUC0-t values compared to single dose. Indeed, at this dose level, the Vdss, clearance and elimination rates appeared to be reduced with repeated dosing. However, increases in Cₘₐₓ and AUC0-t were not seen at higher dose levels, and there was no evidence suggesting accumulation of scyllo-cyclohexanehexyl with repeat dosing. As for single doses, the scyllo-cyclohexanehexyl was absorbed rapidly, with mean tₘₐₓ observed between 1.2 and 1.3 hours post-dose. The t½ values increased slightly from single doses, ranging from 3.1-4.4 hours. Estimates of MRT were consistent.

### Dog

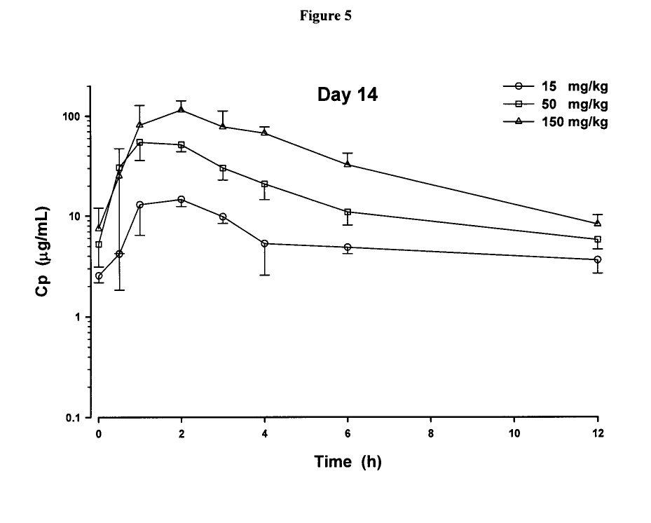

Plasma PK of a scyllo-cyclohexanhexyl in dogs was assessed after 14 days oral dosing of 15, 50 and 150 mg/kg, administered twice daily. Plots of the mean plasma concentrations versus time are presented in FIG. 5. Derived PK parameters are summarised in Table 6. As on Day 1 of this study Cₘₐₓ and AUC values increased proportionally with dose level of scyllo-cyclohexanehexyl. However, dose-normalised exposures appeared to decrease with dose. This contrasts with positive trend observed after acute exposure. There was no evidence suggesting accumulation of the scyllo-cyclohexanehexyl with repeat dosing. As for single doses, the scyllo-cyclohexanehexyl was absorbed rapidly, with tₘₐₓ observed between 1.1 and 1.8 hours post-dose. The t½ values increased slightly from single doses, ranging from 2.9-4.7 hours. Estimates of MRT were consistent. The Vdss and Cl/F values were similar for all dose levels.

### Brain Penetrance

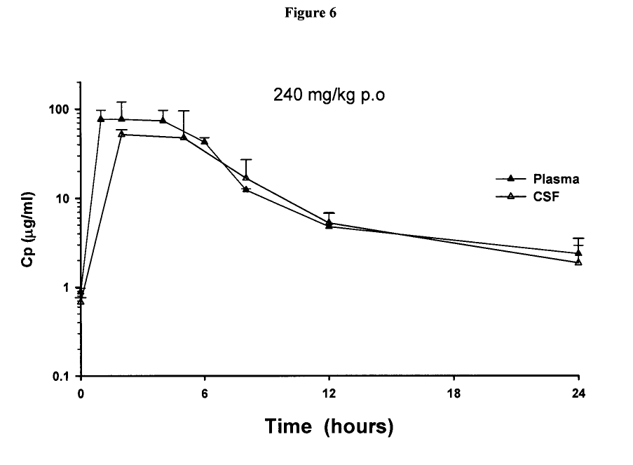

Dogs in the single dose study (exploring doses of 20, 80, 240 mg/kg) were fitted with catheters to permit serial collection of cerebrospinal fluid (CSF) samples. CSF bathes the central nervous system and sits on the inside of the blood brain barrier. Although a centrally-acting drug will still need to penetrate brain tissue to exert an effect, quantification of drug in CSF may provide a gross estimate of brain-penetrance. Plots of the mean CSF and plasma concentrations following oral administration of 240 mg/kg are presented in FIG. 6. PK parameters for CSF and for plasma, derived from the same dogs, are presented in Table 7.

Penetration of the scyllo-cyclohexanehexyl into the brain was extensive and rapid. The tₘₐₓ values observed in CSF ranged from 2.8-4.0 hours. These values were very similar to the plasma tₘₐₓ values derived from the same animals (2.3-2.8 hours). This suggests a very rapid passage for the scyllo-cyclohexanehexyl across the blood brain barrier. Mean Cₘₐₓ values in CSF ranged from 27-66% of the plasma Cₘₐₓ values observed in the same animals. Similarly, the AUC_{0-Inf} in CSF averaged 62% or 65% of that observed in plasma in the same animals, suggesting that scyllo-cyclohexanehexyl has a high brain penetration.

CSF was also collected from dogs at the end of the 14 day repeat study upon necropsy (approximately 24 hours after the final dose was administered). Mean levels of the scyllo-cyclohexanehexyl in these samples are listed in Table 8. Levels of the scyllo-cyclohexanehexyl in CSF increased with dose and followed the profile observed in plasma.

### Pharmacokinetic Summary

The PK of the scyllo-cyclohexanehexyl was strongly consistent between rats and dogs. Maximum plasma concentrations and exposures were broadly dose-proportional in both species. Following repeated oral administration of 150 mg/kg, twice daily, AUC₀₋ₜ values of 573 and 523 μg/h/mL were obtained from rats and dogs respectively. In both species the scyllo-cyclohexanehexyl was absorbed rapidly, had a short half-life, and a very large volume of distribution. Deviation from linearity was observed in both species. In each case this manifested as a decrease in clearance and volume of distribution after repeat administration of a low dose level (15 mg/kg) of the scyllo-cyclohexanehexyl. No such changes were observed at higher dose levels in either species. No accumulation was observed after chronic exposure.

Comparison of PK profiles following oral and i.v. administration in dogs suggested a very extensive oral absorption with negligible first-pass effect. The estimated bioavailability is >90%. Scyllo-cyclohexanehexyl is therefore well-suited as an orally-administered therapy in the clinic. Twice daily administration of the scyllo-cyclohexanehexyl may be most suitable.

The scyllo-cyclohexanehexyl crossed the blood-brain-barrier extremely effectively in dogs. The scyllo-cyclohexanehexyl is therefore likely to reach its site of action at high concentrations in Alzheimer's patients, and will thus be able to exert its target pharmacological effect.

### Effective Concentration/Dose Level

In vitro work has demonstrated that there is likely to be a stoichiometric relationship between amyloid species and scyllo-cyclohexanhexyl levels required for efficacy. A 25:1 stoichiometric relationship (scyllo-cyclohexanhexyl:amyloid peptide) was the lowest observed to have any impact on synthetic Aβ fibrils. When naturally-expressed oligomers were investigated at 1-2 nM (levels considered to be relevant for AD: Cleary, J. P., et al., (2005), Nature Neuroscience, 8: 79-84), the lowest concentration of scyllo-cyclohexanhexyl showing any effect was 125 nM, suggesting that a 100:1 relationship was effective at altering the oligomeric profile of Aβ. However, at 5 μM (0.9 μg/mL) the scyllo-cyclohexanehexyl showed greater consistency of effect in this regard.

More complex systems provide support for 5 μM (0.9 μg/mL) as an effective local concentration of the scyllo-cyclohexanehexyl. In long term potentiation experiments, 5 μM was the lowest maximally-effective dose level (again, with amyloid oligomer concentration at 1-2 nM). The effectiveness of this concentration was confirmed in acute cognitive dysfunction study in rats.

In the clinic, effective local concentrations of a scyllo-cyclohexanehexyl will need to be achieved following oral dosing. Three studies have examined the effective dose range of a scyllo-cyclohexanehexyl following oral dosing. In young CRND8 mice (dosed 12-16 weeks of age, at a time when symptoms are new), 3.3 mg/kg/day provided efficacy in terms of behaviour and Aβ accumulation. In slightly older CRND8 mice (5-6 months), 30 mg/kg/day showed maximum effectiveness at reducing plaques. In rats infused intracerebroventricularly (icy) with All oligomers, approximately 30 mg/kg/day (the lowest dose administered orally) provided effective alleviation of cognitive dysfunction. Therefore, considering 2 species, different animal ages and different endpoints, a dose level ranging between 3-30 mg/kg/day has consistently shown efficacy.

The levels of the scyllo-cyclohexanehexyl in the brain of animals in these studies have not been quantified. However, pharmacokinetic studies in dogs have shown that penetration of the scyllo-cyclohexanehexyl into the brain was extensive and rapid. Mean Cₘₐₓ and AUC_{0-Inf} values in CSF averaged 60% of that observed in plasma. In dogs that had received 30/mg/kg/day scyllo-cyclohexanehexyl for 14 days, levels of scyllo-cyclohexanehexyl in the CSF were ˜4 μg/mL in both plasma and CSF (12 and 24 hours after the last administration). In general, there is great consistency between the pharmacokinetic profiles of rats and dogs. The mean plasma concentration of scyllo-cyclohexanehexyl 12 hours after the last administration in a 28 day rat study was ˜6 μg/mL. Therefore it is likely that the animals receiving 30 mg/kg/day in the rat efficacy experiment discussed above had CSF levels in a similar range. It is anticipated that there will be differences between drug concentrations in CSF and at the site of action in the brain. Nonetheless, these levels are broadly consistent with the in vitro estimates of lowest effective concentration (0.9 μg/mL).

Considering the various preclinical studies together, it seems likely that probability of a scyllo-cyclohexanehexyl providing efficacy in AD patients will be maximised by maintaining the level of the compound in CSF in μg/mL range. Dose levels ranging between 3-30 mg/kg/day should be sufficient to achieve μM concentration of a scyllo-cyclohexanhexyl in the brain.

### Example 2

In TgCRND8 mice, administration of scyllo-cyclohexanehexyl can prevent and limit the Alzheimer's-like phenotype which these animals express. Scyllo-cyclohexanehexyls are more effective than the -related stereoisomer, myo-cyclohexanehexyl, in this regard. The putative site of action of a scyllo-cyclohexanehexyl in Alzheimer's disease models is the brain. The effect of treatment with a scyllo-cyclohexanehexyl or myo-cyclohexanehexyl on brain levels of these molecules was therefore investigated. In untreated animals, myo-cyclohexanehexyl was more abundant than scyllo-cyclohexanehexyl in brain and CSF. Ad libitum administration of a scyllo-cyclohexanehexyl provided major increases in levels of this molecule in the brain and CSF. In contrast, administration of myo-inositol provided only a minor increase in brain levels of this molecule, and significantly decreased scyllo-cyclohexanehexyl brain exposure. A scyllo-cyclohexanehexyl therefore holds significantly greater potential than myo-cyclohexanehexyl as a centrally-acting pharmaceutical agent in mice. Scyllo-cyclohexanehexyl was not incorporated into phosphatidylinositol lipids in the brain, and did not influence the incorporation of other inositols, suggesting that signal transduction pathways should be unaffected.

### Objective

To provide information on the impact of scyllo-cyclohexanehexyl treatment on levels of inositol in CSF and brain of TgCRND8 mice.

### Materials and Methods

### Materials

Mice: TgCRND8; Age: 4 months

### Study Drugs:

Scyllo-cyclohexanehexyl supplied by Hokko (Japan)
Myo-cyclohexanehexyl: supplied by Hokko (Japan)

### Dosage:

Study drugs were administered either at 30 mg/kg by oral gavage, or ad libitum by dissolution in drinking water. For administration in drinking water, study drugs were dissolved at 10 mg/ml. The average amount of water taken from the bottle was 3 ml daily per animal. The average mouse weight was 33 g. Thus for ad libitum dosing the average dose of study drug approached 900 mg/kg over 24 hours.

### Methods

Quantification of scyllo-cyclohexanehexyl and myo-inositol. Scyllo-cyclohexanehexyl and myo-cyclohexanehexyl levels in the brain and CSF were analyzed using gas chromatography/mass spectrometry. D-chiro-inositol was used as an internal control, as it is not present in these biological samples.
Derivatization: To increase the volatility and thermal stability of these compounds and to allow for proper peak separation, these samples need to first be derivatized. The derivatization was adapted from a method presented previously (Shetty, H U, et al, 1995, Anal. Biochem. 224:279-85), where the hydroxyl groups of the cyclohexanehexyl were replaced with acetyl groups. Briefly, half a mouse brain was homogenized in 2×2 ml of methanol. The resulting suspension was centrifuged (5 min, 5000×g) and the equivalent of 30 mg of brain tissue was placed in a glass tube with a Teflon-lined cap. Similarly, a 5-15 μl sample of CSF was mixed with 1 ml of methanol and allowed to stand at room temperature for 5 minutes. The resulting suspension was centrifuged (5 min, 5000×g) and the supernatant transferred to a glass tube (Teflon-lined cap). In each case D-chiro-inositol was then added to the tube at a set concentration (50 ng/1.11 for brain, 1 ng/μl for CSF). These samples were then evaporated to complete dryness (Speedvac; 60° C.). The residue was treated with 100 μl of pyridine reagent (1 mg/ml 4-dimethylaminopyridine solution in pyridine) and 100 μl of acetic anhydride. This mixture was flushed with dry nitrogen, securely capped and the tubes heated (80° C.; 30 min).

### Analysis:

Following derivatization, samples were cooled (RT) and the unreacted acetylating reagent evaporated using a steady stream of nitrogen gas. The derivatized products were re-dissolved (4 ml; hexane-ethyl acetate (80:20, v/v)) and washed with 1 ml of 5% sodium hydrogencarbonate solution. Following vortexing (5 min) and centrifugation (3 min, 1000×g), the organic layer was transferred to another tube and evaporated (Speedvac; 40° C.). The residue was reconstituted in 100 μl; ethyl acetate. 1 μL of this was injected into the GC/MS system. A similar procedure was followed using stock concentrations of scyllo- and myo-cyclohexanehexols, which were then used to generate concentration curves (in this case the internal standard was derivatized separately and then added).

GC/MS was performed using a Perkin Elmer TurboMass Autosystem XL, with a quadrupole mass spectrometer and electron ionization. Gas chromatography was performed using a 30 m×0.25 mm×0.25 μm ZB 5 (5% diphenyl/95% dimethylpolysiloxane) column, using the carrier gas Helium (1 ml/min). Samples were injected with the split set to 50 at 1 min and 0 at 5 min, the injector temperature was set at 300° C. and an initial oven temperature of 80° C. After a hold of 1 min, the temperature was ramped at 45° C./min to 187° C. and held there for 15 min. The oven was then ramped at 45° C./min to 295° C. and held for 1.5 min. Sample peaks were analyzed using selected-ion monitoring, using m/z 168 for determining cyclohexanehexyl levels in the brain and CSF and m/z 373 for determining cyclohexanehexyl levels in plasma, as monitoring the larger ion reduced noise. Sample peak areas were compared to the concentration curves.

### Lipid Extraction and Hydrolysis

The method used for lipid isolation and analysis was adapted from a previously presented method (Kersting, M C et al, 2003, J. Eukaryot. Microbiol. 50: 164-168). Briefly, half a mouse brain was homogenized in 2 ml of dH₂O and 500 μl of that homogenate was used for lipid isolation. To isolate lipids and polyphosphoinositol, that 500 μl was placed into a glass screw-cap tube containing 3.75 ml of chloroform/methanol/conc. HCl (10:20:0.1, v/v) and vortexed. 1.25 ml of chloroform and 1.25 ml of 0.1 M HCL were added and the solution was vortexed again. Samples were then centrifuged (200 g) to separate the phases. The organic phase, containing the lipids, was dried under nitrogen gas, resuspended in 200 μl of chloroform/methanol (6:1, v/v) and streaked onto a silica gel plate. The plate was placed in hexane/ethyl ether/acetic acid (70:30:1, v/v). Once the solvent had migrated within 1 cm from the top of the plate, the plate was removed from the TLC tank and air dried. The origin, containing phospholipids, was collected and the lipids eluted using four 1 ml washes of chloroform/methanol/conc. HCl (2:1:0.1, v/v). The lipids were dried under nitrogen gas and redissolved in 1 ml of 6 N HCl. The sample was acid hydrolyzed (110° C., 56 h) to release inositol from inositol-containing phospholipids. The hydrolysate was dried under nitrogen gas and derivatized (as above) prior to GCMS analysis. Sample peaks were analyzed using selected-ion monitoring, using m/z 168.

### Study Design

Brain and CSF levels of scyllo-cyclohexanehexyl and myo-inositol following one month's ad libitum dosing with scyllo-cyclohexanehexyl or myo-cyclohexanehexyl: Mice received ad libitum dosing of scyllo-cyclohexanehexyl or myo-cyclohexanehexyl for 1 month, prior to obtaining CSF samples (n=5 animals treated with scyllo-cyclohexanehexyl and 10 animals treated with myo-cyclohexanehexyl) and brain samples (n=5 animals treated with scyllo-cyclohexanehexyl and 15 animals treated with myo-cyclohexanehexyl). Samples were also obtained from untreated animals (CSF n=10, brain n=17).
Incorporation of scyllo-cyclohexanehexyl into phosphatidylinositol lipids within the brain: five TgCRND8 mice received ad libitum dosing for 1 month (the same animals that provided CSF and brain samples after ad libitum dosing; above). Brain tissue was then harvested and lipids examined.

### Results

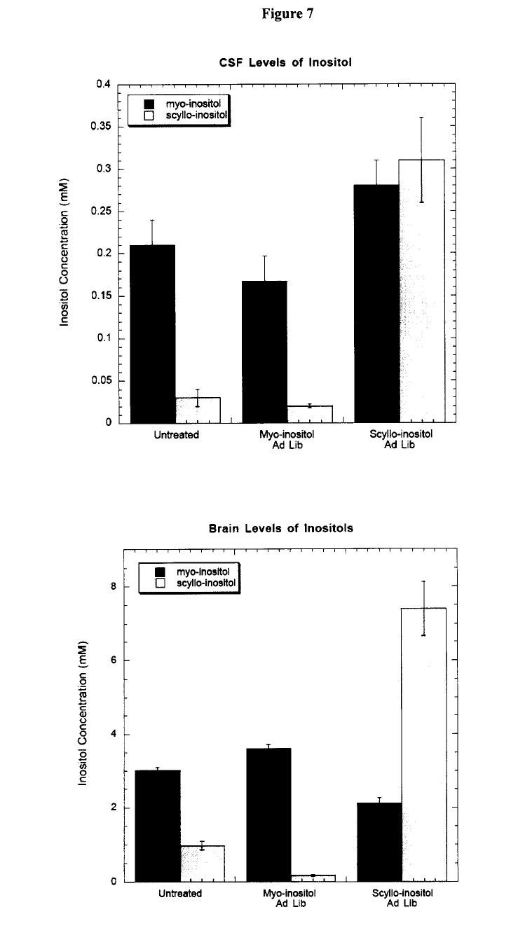

Brain and CSF levels of scyllo-cyclohexanehexyl and myo-cyclohexanehexyl following one month's ad libitum dosing with scyllo-cyclohexanehexyl or myo-cyclohexanehexyl. Mice received ad libitum dosing (study drug in drinking water) of scyllo-cyclohexanehexyl or myo-cyclohexanehexyl for one month. Levels of scyllo-cyclohexanehexyl and myo-cyclohexanehexyl were then determined in CSF and brain (FIG. 7). In untreated animals, scyllo-cyclohexanehexyl and myo-cyclohexanehexyl were quantifiable in both CSF and brain tissue. Myo-cyclohexanehexyl was more abundant than scyllo-cyclohexanehexyl in both cases (7-fold higher in brain, 3-fold higher in CSF). These observations are consistent with observations in humans (Michaelis, 1993). Animals that had received scyllo-cyclohexanehexyl ad libitum for one month had profoundly increased levels of scyllo-cyclohexanehexyl in CSF (10-fold increase; p<0.001) and brain (7-fold increase; p<0.001). CSF levels of myo-cyclohexanehexyl were not significantly different from untreated animals (p=0.7). Levels of myo-cyclohexanehexyl in brain were significantly lower than observed in untreated animals (p<0.001), although the magnitude of this change was minor (30% decrease). In contrast to observations following administration of scyllo-cyclohexanehexyl, animals that had received myo-cyclohexanehexyl ad libitum for one month did not show any significant changes in the levels of myo-cyclohexanehexyl in CSF (p=0.35). The level of myo-cyclohexanehexyl in the brain was significantly increased (p<0.001) although the magnitude of this change was minor (20% increase). CSF levels of scyllo-cyclohexanehexyl were not significantly different from untreated animals. Levels of scyllo-cyclohexanehexyl in brain were 80% lower than observed in untreated animals (p=0.008). Myo-cyclohexanehexyl therefore inhibits the distribution of scyllo-cyclohexanehexyl into brain tissue.

Both scyllo-cyclohexanehexyl and myo-cyclohexanehexyl interact with amyloid-β in vitro, and can prevent amyloid-β-induced neurotoxicity. The most effective site of action for a drug that targets amyloid-β to treat Alzheimer's disease is the brain. FIG. 7 shows that administration of scyllo-cyclohexanehexyl provided major increases in levels of this drug in the brain and CSF, and therefore supports the further development of this agent. In contrast, administration of myo-cyclohexanehexyl provides only a minor increase in brain levels of this molecule, and significantly decreases scyllo-cyclohexanehexyl brain exposure. These observations suggest that scyllo-cyclohexanehexyl holds significantly greater potential than myo-cyclohexanehexyl as a centrally-acting pharmaceutical agent in mice.

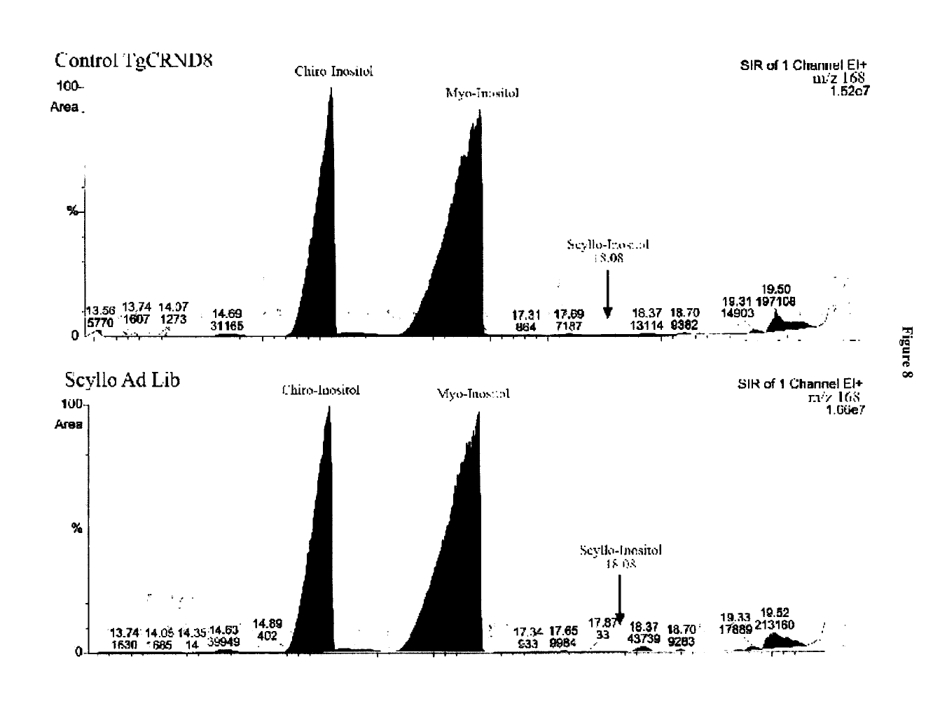

Incorporation of scyllo-cyclohexanehexyl into phosphatidylinositol lipids within the brain. Phosphatidylinositol lipids are essential components of signal transduction pathways. Phosphatidylinositol lipids were therefore isolated from the brains of mice that had received one month's ad libitum administration of scyllo-cyclohexanehexyl to determine whether scyllo-cyclohexanehexyl was incorporated (FIG. 8). Phosphatidylinositol lipids from untreated mice contained significant amounts of chiro- and myo-cyclohexanehexyl. However, no scyllo-cyclohexanehexyl was detectable. The profile of phosphatidylinositol lipids from mice that had received ad libitum dosing with scyllo-cyclohexanehexyl for one month was identical to untreated animals. No scyllo-cyclohexanehexyl was incorporated into the lipids, despite the high levels of this molecule in the brain. Furthermore, the levels of chiro- and myo-cyclohexanehexyl in the lipids were unaffected. These data suggest that the scyllo-cyclohexanehexyl administration does not influence the constituents of phosphatidylinositol lipids, and thus signal transduction pathways should not be affected.

### Conclusion

In untreated animals, myo-cyclohexanehexyl was more abundant than scyllo-cyclohexanehexyl in brain and CSF.
Ad libitum administration of scyllo-cyclohexanehexyl provided major increases in levels of this drug in the brain and CSF. In contrast, administration of myo-cyclohexanehexyl provided only a minor increase in brain levels of this molecule, and significantly decreased scyllo-cyclohexanehexyl brain exposure. Scyllo-cyclohexanehexyl therefore holds significantly greater potential than myo-cyclohexanehexyl as a centrally-acting pharmaceutical agent in mice.
Scyllo-cyclohexanehexyl was not incorporated into phosphatidylinositol lipids in the brain, and did not influence the incorporation of other inositols, suggesting that signal transduction pathways should be unaffected.

### Example 3

### Summary

Previously, prophylactic and therapeutic dosing of scyllo-cyclohexanehexyl, was shown to be effective in limiting the Alzheimer's-like phenotype which develops in TgCRND8 mice. A dose of 3.3 mg/kg/day, administered by oral gavage twice daily, showed significant alleviation of cognitive dysfunction and reduction in plaque burden. In contrast, 1.0 mg/kg/day showed partial effectiveness, and the 0.3 mg/kg/day had no observed effect, compared to untreated animals. In this study higher dose levels of scyllo-cyclohexanehexyl were administered to identify a maximally effective dose level of scyllo-cyclohexanehexyl in 4 month TgCRND8 mice. The dose levels 3.3, 10 and 30 mg/kg/day all showed significant alleviation of cognitive dysfunction. Assessment of plaque burden is ongoing. This study suggests that 3.3 mg/kg/day may be a maximally effective dose level in limiting Alzheimer's-like pathology in TgCRND8 mice, aged 4 months.

### Objectives

To determine the maximally effective dose level of scyllo-cyclohexanehexyl in 4 month TgCRND8 mice.

### Materials and Methods

### Mice:

Description: TgCRND8 mice express a human amyloid precursor transgene (APP₆₉₅) bearing two missense mutations that are associated with AD in humans (KM670/671NL and V717F). At about three months of age, the TgCRND8 mice display progressive spatial learning deficits that are accompanied both by rising cerebral Aβ levels and by increasing numbers of cerebral extracellular amyloid plaques (Chishti M A, 2001, J. Biol. Chem. 276:21562-21570). By six months of age, the levels of Aβ and the morphology, density and distribution of the amyloid plaques in the brain of TgCRND8 mice are similar to those seen in the brains of humans with well-established AD. As in human patients with AD, the biochemical, behavioural and neuropathological features of the mouse model are accompanied by accelerated mortality.
Sample size:

| | |
|---|---|
| Placebo | n = 9 |
| 3.3 mg/kg/day | n = 5 |
| 10 mg/kg/day | n = 8 |
| 30 mg/kg/day | n = 9 |

Age: 3 months old at commencement of dosing

### Study Drugs:

Scyllo-cyclohexanehexyl supplied by Hokko (Japan).
Placebo (sterile water)

### Dosage and Scheduling:

TgCRND8 mice were treated with scyllo-cyclohexanehexyl by oral gavage from 3 to 4 months. Dose levels administered were 3.3, 10 and 30 mg/kg/day, or placebo, administered as two equal doses per day. Animals were assessed at 4 months of age.

### Methods

Behavioural tests: Morris Water Maze testing was performed as previously described (Janus C, et al., 2000, Nature 408:979-982). After non-spatial pre-training, mice underwent place discrimination training for 5 days with 4-trials per day. Data were subjected to a repeated measures analysis of variance (ANOVA) with treatment (untreated, scyllo-cyclohexanehexyl) as the between subject factor.
Cerebral plaque burden. Brains were removed and one hemisphere was fixed in 4% paraformaldehyde and embedded in paraffin wax in the mid sagittal plane. To generate sets of systematic uniform random sections, 5 μm serial sections were collected across the entire hemisphere. Sets of sections at 50 μm intervals were used for analyses (10-14 sections/set). Plaques were identified after antigen retrieval with formic acid, and incubation with primary anti-Aβ antibody (Dako M-0872), followed by secondary antibody (Dako StreptABCcomplex/horseradish kit). End products were visualized with DAB and were counter-stained with luxol fast blue. Aβ plaque burden was assessed with Leco IA-3001 image analysis software interfaced with Leica microscope and Hitachi KP-M1U CCD video camera. Openlab imaging software (Improvision, Lexington, Mass.) was then used to convert micrographs to binary images for plaque number and plaque area determinations. Vascular Aβ burden was defined as Aβ plaques originating from or surrounding blood vessels and was analysed similarly.

### Results

Morris Water Maze Test of Spatial Memory: TgCRND8 mice were treated with 3.3, 10 and 30 mg/kg/day scyllo-cyclohexanehexyl or placebo from 3 to 4 months of age. At the end of the treatment period, the Morris Water Maze was used to evaluate cognitive function. In this assay animals are placed on consecutive days into a pool with a submerged platform. The total swim path length to find the hidden platform is evaluated. With incremental daily experience, the swim path length typically decreases.

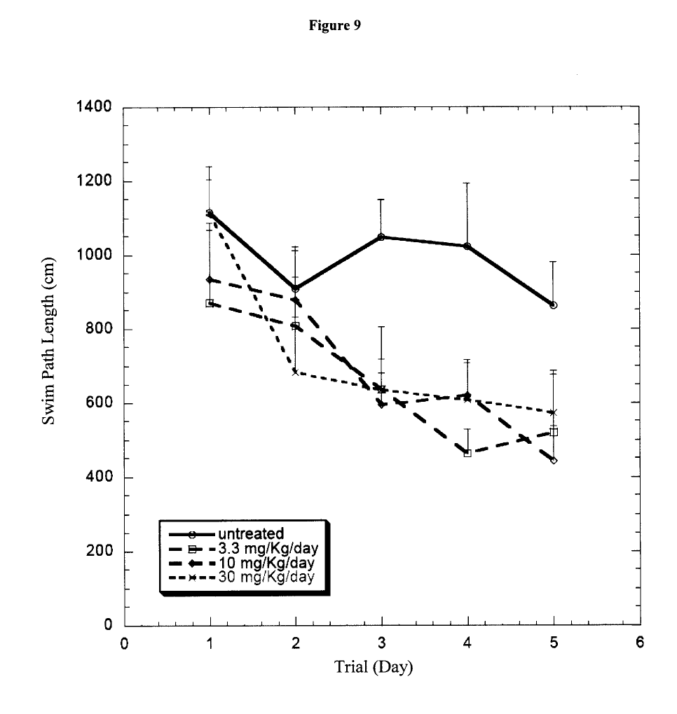

After 5 days of assessment, swim path length was significantly shorter for animals that had received scyllo-cyclohexanehexyl (all dose levels) compared to animals that had received placebo (FIG. 9, p=0.003, 0.008, 0.002 for 3.3, 10 and 30 mg/kg/day, respectively). Dose levels from 3.3-30 mg/kg/day showed equivalent effects in swim path length. Thus 3.3 mg/kg/day appeared to be a maximally efficacious dose level as assessed by this assay under the conditions tested (i.e. animals aged 4 months). This outcome is consistent with a study where 3.3 mg/kg/day showed cognitive benefit that was equivalent to ad libitum treated animals from a separate study.

### Conclusion

Scyllo-cyclohexanehexyl was effective at alleviating the cognitive dysfunction observed in TgCRND8 mice, aged 4 months. Dose levels of 3.3, 10 and 30 mg/kg/day scyllo-cyclohexanehexyl, administered orally twice daily, showed similar efficacy. There was no suggestion of any difference in effect between 3.3 mg/kg/day and the higher dose levels. Thus 3.3 mg/kg/day appears to be a maximally efficacious dose level as assessed by this assay under the conditions tested (i.e., animals aged 4 months).

### Example 4

A unitary dosage form of a scyllo-cyclohexanhexyl compound may be dissolution tested by placing it in a paddle-equipped USP-2 apparatus containing 900 ml of a test solution containing the compound at a temperature of 37° C., with the paddle stirring at 50 rpm. If the dosage form is a capsule, it is tested in the same manner except that the test solution may also contain 0.1 mg/mL of trypsin. Filtered aliquots (typically 2 or 10 mL) of the dissolution medium are taken at various times or pull points. An aliquot is filtered and assayed for cyclohexane polyalcohol compound content utilizing an HPLC assay or other suitable assay. The data is plotted as mgA cyclohexane polyalcohol compound (active cyclohexane polyalcohol compound) released (or % by weight cyclohexane polyalcohol compound base released) on the y-axis vs time on the x-axis. The time at which a selected amount (e.g. 80% by weight) of the cyclohexane polyalcohol compound dose is released is noted. Repeated separate dissolution tests should be conducted and the rates determined and averaged. The described method provides a clear test of the rate of drug release which is independent of the mechanism of cyclohexane polyalcohol compound release from the dosage form.

### Example 5

### Dose Studies

### TgCRND8 Mice: Therapeutic Dosing

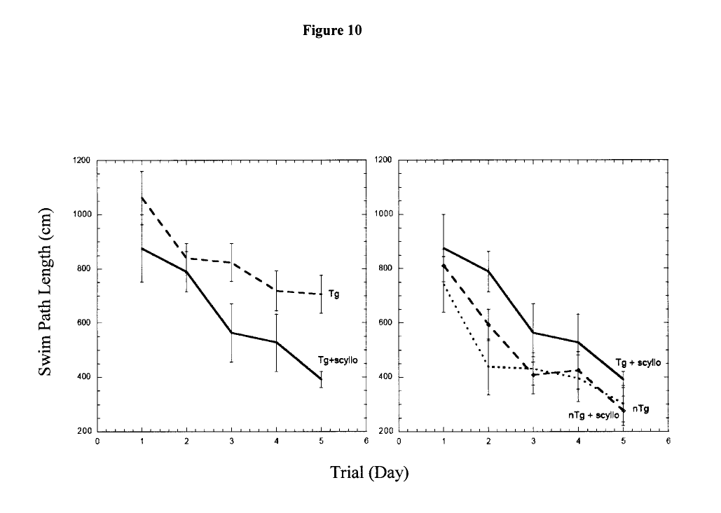

To determine if a scyllo-cyclohexanehexyl compound could abrogate a well-established AD-like phenotype, treatment was delayed until 5 months of age, by which time animals typically have significant cognitive deficits, accompanied by profuse Aβ peptide and plaque burden. Animals were treated for 28 days with AZD-103 in drinking water (10 mg/ml, approximate dose=900 mg/kg over 24 hours). Animals were evaluated at 6 months for cognitive function, amyloid accumulation, plaque burden and synaptic density.
Improvement of cognitive function: The treatment resulted in significantly better behavioural performance (decreased swim path length in the Morris Water Maze), compared to untreated controls (FIG. 10, p=0.01). Indeed swim path lengths for TgCRND8 mice treated with the compound for 28 days were indistinguishable from non-transgenic littermates (p=0.11).
Reduction in amyloid accumulation in the brain: The therapeutic dosing regimen was associated with reduced levels of insoluble Aβ40 and Aβ42 in the brain (Table 9, p<0.05 each).
Reduction in plaque burden: The therapeutic dosing regimen was associated with significantly reduced plaque burden (area and size, Table 9, p<0.05 each)
Increase in synaptic density: The therapeutic dosing regimen was associated with increased synaptic density (p<0.001).

The observations made in a prophylactic dosing context have been extended to the therapeutic context. This is of great importance for a number of reasons. First, the alleviation of cognitive deficit, and the reductions of amyloid and plaque burden can be achieved at a time when these disease characteristics are already well advanced. Second, the therapeutic context is more relevant for the human disease where patients will be treated once diagnosed. Third, reduction in amyloid accumulation and plaque burden represents a limitation of the underlying cumulative pathology of the disease. The compound is therefore acting as a disease modifying agent, rather than just influencing symptoms.

### TgCRND8 Mice: Effective Dose Range

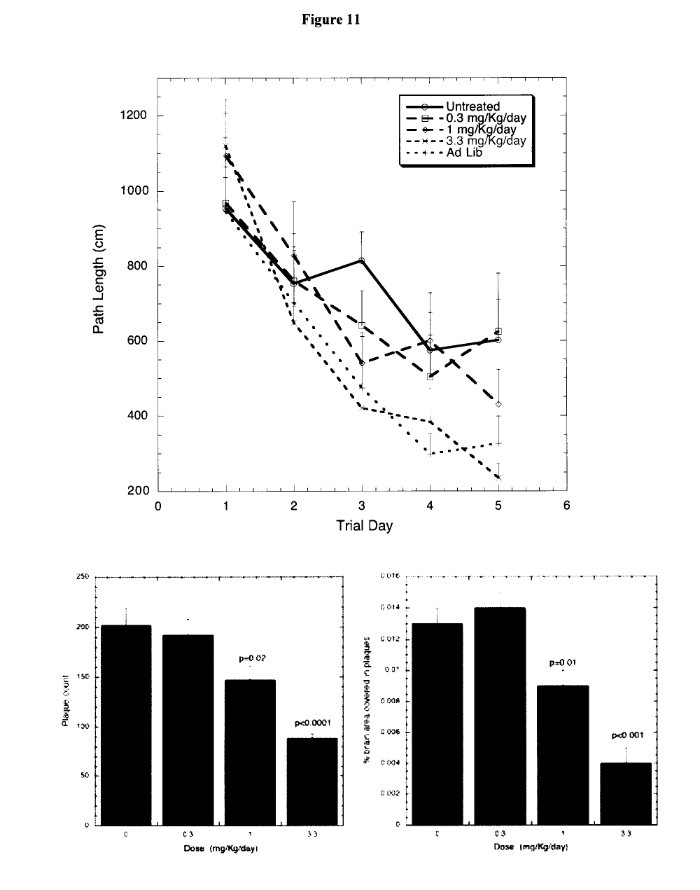

The highest possible dose level of scyllo-cyclohexanehexyl compound was administered in a CRND8 mouse model. Once the broad range of clinically-relevant activities had been demonstrated and replicated, the effective dose range of the compound could then be explored. TgCRND8 mice were dosed for 28 days from 3 to 4 months of age, i.e. a time when the disease phenotype would be newly established. This study design was selected to enhance the efficiency of the study (dose earlier) and maximise the information obtained (dose at a time when changes are of sufficient magnitude to detect a treatment effect). Animals were administered total daily doses of 0.3, 1 or 3.3 mg/kg/day or vehicle, administered in 2 equal doses per day, by oral gavage. Cognitive function and plaque burden were assessed.
Improvement of cognitive function: Animals were assessed using the Morris Water Maze at 4 months (FIG. 11). The 3.3 mg/kg/day dose group showed a significantly shorter swim path length on day 5 of training, compared to untreated animals (p=0.04). This path length was similar to that observed in previous experiments in mice dosed with the compound in drinking water ad libitum. This suggests that the 3.3 mg/kg/day may be approaching a maximal effect under these experimental conditions as assessed by this endpoint. In contrast, the path length in the 0.3 mg/kg/day cohort was indistinguishable from untreated controls.
Reduction in plaque burden: Animals were assessed for plaque burden (plaque count and brain plaque area: FIG. 11). Plaque parameters in animals receiving 0.3 mg/kg/day were indistinguishable from untreated controls. In contrast, both parameters were significantly reduced by the treatment at 1 mg/kg/day (plaque count p=0.02, plaque area p=0.011) and 3.3 mg/kg/day (plaque count p<0.0001, plaque area p<0.001). 3.3 mg/kg/day provided the greatest reduction in plaque burden, which was significantly greater than that provided by 1 mg/kg/day (p=0.03 for both parameters). Thus a clear dose response was observed.

The 3.3 mg/kg/day dose has therefore demonstrated significant activity that is consistent between two independent endpoints: cognition and plaque burden. This low dose level may therefore be considered the lowest efficacious dose in an animal model of early disease.

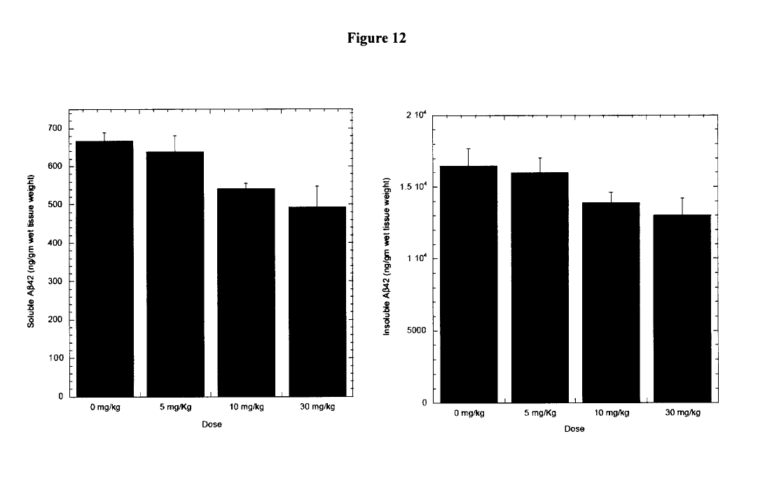

Dose response was further examined in animals dosed from 5-6 months of age. Disease is more advanced at this later time point, so there may be greater clinical relevance. The dose levels investigated were 5, 10 and 30 mg/kg or vehicle, once daily. Accumulation of Aβ42 was assessed.
Reduction in amyloid accumulation in the brain: Levels of Aβ42 in brain homogenates were assessed by ELISA. A dose response was suggested. Both 10 and 30 mg/kg/day of compound provided significant reductions in levels of soluble Aβ42 in the brain, compared to vehicle (FIG. 12, 10 mg/kg/day: p=0.03; 30 mg/kg/day: p=0.02), with 30 mg/kg/day apparently showing the greatest effect.

A dose response has therefore been defined and replicated across two independent investigations.

### Example 6

Inositol is a simple polyol with eight naturally occurring stereoisomers. Myo-inositol, D-chiro and epi-inositol have been examined as potential therapeutic agents for various diseases, with favorable results, but treatment with scyllo-inositol has not been previously investigated. scyllo-Inositol has been shown to inhibit cognitive deficits in TgCRND8 mice and significantly ameliorate disease pathology, suggesting it might be effective in treating Alzheimer's disease (AD). In this study, scyllo-inositol is shown to have a sustained ability to treat animals at advanced stages of AD-like pathology. Significant decreases in insoluble Aβ40 Aβ42, and plaque accumulation were observed in the brains of treated versus untreated TgCRND8 mice. The growth of plaques of all sizes was inhibited by scyllo-inositol administration. To demonstrate that the scyllo-inositol effects were within the CNS, gas chromatography/mass spectrometry was used to examine myo and scyllo-inositol concentrations after oral administration. Further, how closely scyllo- and myo-inositol are inter-regulated in the CNS and whether scyllo-inositol, if elevated within the CNS, would incorporate into phosphatidylinositol lipids were examined. Cerebral spinal fluid levels of scyllo-inositol increased after scyllo-inositol treatment but not myo-inositol treatment. scyllo-Inositol treatment also caused increased levels of scyllo-inositol in the brain. scyllo-Inositol, even at elevated levels, does not incorporate into the phosphatidyl-inositol family of lipids. These combined results demonstrate that scyllo-inositol accumulates within the CNS up to tenfold endogenous levels and does not interfere with phosphatidylinositol lipid production.

One of the objectives of the study was to determine whether scyllo-inositol treatment would remain effective at amyloid and Aβ levels equivalent to end-stage sporadic Alzheimer's disease. The second aim was to determine whether inositol oral administration elevates scyllo-inositol concentrations in brain. myo-Inositol is critical for maintaining osmolarity and signal transduction pathways within the CNS and while the physiological concentrations of inositol in the brain and some mechanisms of its regulation are understood, this understanding is still very general [1]. The interconversion between myo- and scyllo-inositol within the brain has not been extensively studied, therefore this study sought to better understand how carefully this system is regulated. More specifically, whether myo- or scyllo-inositol treatment in mice would change the CNS concentrations of both polyols and whether altering the concentration of one could affect the other were examined. In addition whether scyllo-inositol, when CNS concentrations are increased, would incorporate into the phosphatidylinositol family of lipids, thereby altering signal transduction pathways was examined.

The following materials and methods were used in the study.

### Materials and Methods

Mice TgCRND8 mice were maintained on an outbred C3H/C57B16 background [2]. These mice over express Swedish (KM670/671NL) and Indiana (V717F) APP mutations in cis on the APP695 transcript under control of the Syrian hamster prion gene promoter. One group of mice were treated with 10 mg/ml scyllo-inositol ad libitum through their drinking water for 2 months starting at 5 months of age, and the effects of treatment on Aβ and plaque levels were determined. A second group of mice were treated with either myo or scyllo-inositol ad libitum, and changes in inositol levels were quantified. Each mouse typically consumed between 2.5-3 ml of the fluid per day, which is equivalent to a 25-30 mg dose of scyllo-inositol per animal. All experiments were performed according to the Canadian Council on Animal Care guidelines.
Materials All reagents were purchased from Sigma (St. Louis, Mo., USA) or EMD (Merck, Darmstadt, Germany) unless otherwise noted. scyllo-Inositol was acquired from Transition Therapeutics (Toronto, Ontario, Canada).
Cerebral Aβ burden One brain hemisphere was fixed in 4% paraformaldehyde and embedded in paraffin wax. Sets of sections at 50-μm intervals were used for analysis (five sections per set). Plaques were identified using a primary Aβ-specific antibody 6F/3D (Dako, M-0872) and visualized using 3,3-diaminobenzidine (DAB). The Aβ plaque burden was assessed using an Openlab imaging software (Improvision). Micrographs were converted to binary images, and the percent brain area covered in plaques and the plaque size distribution was determined.
Plasma and cerebral Aβ content Hemi-brain samples were homogenized in a buffered sucrose solution, followed by either 0.4% diethylamine and 100-mM NaCl to examine soluble Aβ levels or cold formic acid to examine total Aβ. Samples were neutralized, diluted, and analyzed for Aβ40 and Aβ42 levels using a commercially available ELISA kit (Biosource International). The samples were analyzed in triplicate. A similar method was used for plasma.
Quantification of myo and scyllo-inositol Scyllo and myo-inositol concentrations in the brain, CSF, and plasma were quantified using gas chromatography/mass spectrometry (GC/MS). To increase the volatility and thermal stability of these compounds and to allow for peak separation, these samples were first derivatized. The derivatization protocol was adapted from Shetty et al. [3]. Briefly, one brain hemisphere was weighed and homogenized in 2×2 ml of methanol, and the resulting suspension was centrifuged for 5 min at 5,000×g. A volume of supernatant equivalent to 30 mg of brain tissue (based on the weight of the tissue before homogenization) was analyzed. Similarly, for plasma and CSF, either 100 μl of plasma or 5 μl of CSF were mixed with 1 ml of methanol; the solution was allowed to stand real-time for 5 min, the resulting suspension centrifuged for 5 min at 5000×g, and the supernatant removed. To all supernatants, the internal standard D-chiroinositol (Wako, Osaka, Japan) was added at 50 ng/ml for the brain and plasma, and 1 ng/ml for CSF. These samples were evaporated to dryness (Speedvac; 60° C.); 100 μl of pyridine reagent (1 mg/ml 4-dimethylaminopyridine solution in pyridine) and 100 μl of acetic anhydride were added, and tubes were flushed with nitrogen and heated at 80° C. for 30 min. After derivatization, the unreacted acetylating reagent was evaporated under a steady stream of nitrogen. The derivatized products were re-dissolved (4 ml; hexane-ethyl acetate (80:20, v/v)) and washed with 1 ml of 5% sodium carbonate. After vortexing (5 min) and centrifugation (3 min, 1000×g), the organic layer was evaporated (Speedvac; 40° C.). The residue was reconstituted (100 μl; ethyl acetate), and 1 μL of this was injected into the GC/MS system. A similar procedure was followed using stock concentrations of scyllo- and myo-inositol, which were then used to generate standard concentration curves.

GC/MS was performed using a Perkin Elmer TurboMass Autosystem XL with a quadrupole mass spectrometer and electron ionization. GC was performed using a 30 m×0.25 mm×0.25 mm ZB 5 column (5% diphenyl/95% dimethylpolysiloxane, Phenomenex, Macclesfield, UK), using Helium as the carrier gas (1 ml/min). Samples were injected with the split set to 50 at 1 min and 0 at 5 min; the injector temperature was set at 300° C. and an at initial oven temperature of 80° C. After a hold of 1 min, the temperature of 45° C./min was increased to 187° C. and held for 15 min. The temperature was then increased, 45° C./min to 295° C., and held for 1.5 min. The sample peaks were analyzed using selected-ion monitoring at m/z 168. The sample peak areas were compared to the standard concentration curves.

Lipid extraction and hydrolysis The method for lipid isolation and analysis was adapted from Kertsing et al. [4]. Briefly, one brain hemisphere was homogenized in 2 ml of dH₂O, and 500 μl was used for lipid isolation. The organic extraction of brain lipids was performed by the following procedure: 3.75 ml of chloroform/methanol/HCl (10:20:0.1, v/v) followed by 1.25 ml of chloroform, and 1.25 ml of 0.1 M HCL were added and the solution vortexed. The samples were then centrifuged (200×g) to separate the phases. The organic phase containing the lipids was dried under nitrogen gas and resuspended in 200 μl of chloroform/methanol (6:1, v/v) before streaking onto a silica gel 60 F254 plate (EM Industries, Merck, Darmstadt, Germany). The plate was placed in hexane/ethyl ether/acetic acid (70:30:1, v/v). Once the solvent had migrated within 1 cm from the top of the plate, the plate was removed from the thin-layer chromatography tank and air-dried. The origin containing phosphatidylinositol lipids was collected, and the lipids eluted using four 1-ml washes of chloroform/methanol/concentrated HCl (2:1:0.1, v/v). The lipids were dried under nitrogen, redissolved in 1 ml of 6 NHCl, and the acid hydrolyzed (110° C., 56 h). The hydrolysate was dried under nitrogen and derivatized (as above) before GC/MS analysis. The sample peaks were analyzed using selected-ion monitoring, m/z 168.
Statistical analysis The statistical analysis was performed using the Statistical Package for the Social Sciences 11 for Mac OS X. Groups were compared using a one-way ANOVA. If a significant F score was observed (P<0.05), a Bonferroni post hoc test was used to compare the groups with the statistical significance set at P<0.05.

### Results

### Scyllo-Inositol Treatment Reduces AD Pathology

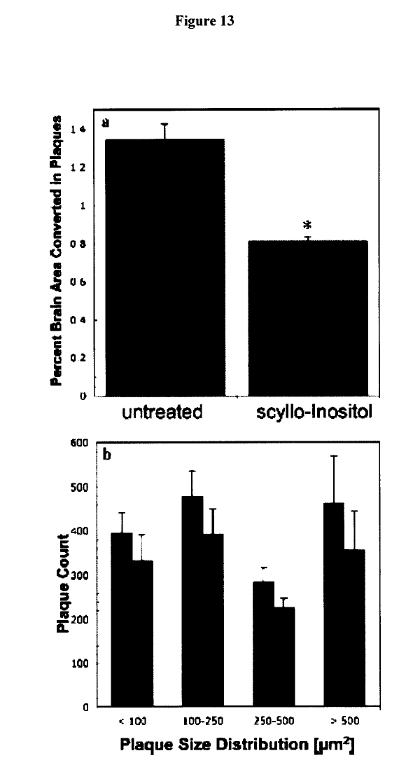

To determine the effects of scyllo-inositol treatment on the end stage of AD-like pathology, 5-month-old TgCRND8 mice were treated for 2 months with scyllo-inositol ad libitum. The end point of this study has Aβ and amyloid loads equivalent to the end-stage disease in humans with sporadic AD [5-7]. The outcome measures analyzed were the soluble and insoluble Aβ40 and Aβ42 in the brain and plasma, the percent brain area covered in plaques, and the plaque size distribution in treated animals compared to the untreated controls. scyllo-Inositol treatment resulted in a significant reduction in insoluble Aβ40 and Aβ42 levels in the brain when compared to the age-matched controls (F_{1,14}=12.012; P<0.05; Table 10). Both soluble Aβ40 and Aβ42 levels in the brain and in the plasma showed a non-significant decrease after treatment. The scyllo-inositol treatment also significantly reduced the percent brain area covered in plaques (F_{1,14}=31.281; P=0.0001; FIG. 13a). To determine whether the decreased plaque load was the result of inhibition of new plaque formation and/or a decrease in established plaque growth, the distribution of plaque size as a result of treatment were analyzed. The findings indicate that scyllo-inositol treatment was effective at inhibiting the growth of plaques of all sizes (FIG. 13b).

### Quantification of Inositol

To determine how oral administration of myo and scyllo-inositol influences the concentration of the two substances in the CSF and in the brain, GC/MS was used to analyze changes in myo and scyllo-inositol levels in the CSF and brain tissue. Both the sodium myo-inositol transporter 1 (SMIT-1) and the hydrogen myo-inositol transporter are known to transport scyllo-inositol in vitro [15, 25]. SMIT-1 is known to be constitutively active, has an affinity for both myo and scyllo-inositol and is expressed at the choroid plexus; therefore, scyllo-inositol oral administration would be expected to alter CNS inositol levels. myo-Inositol oral administration would also be expected to alter CNS inositol levels.

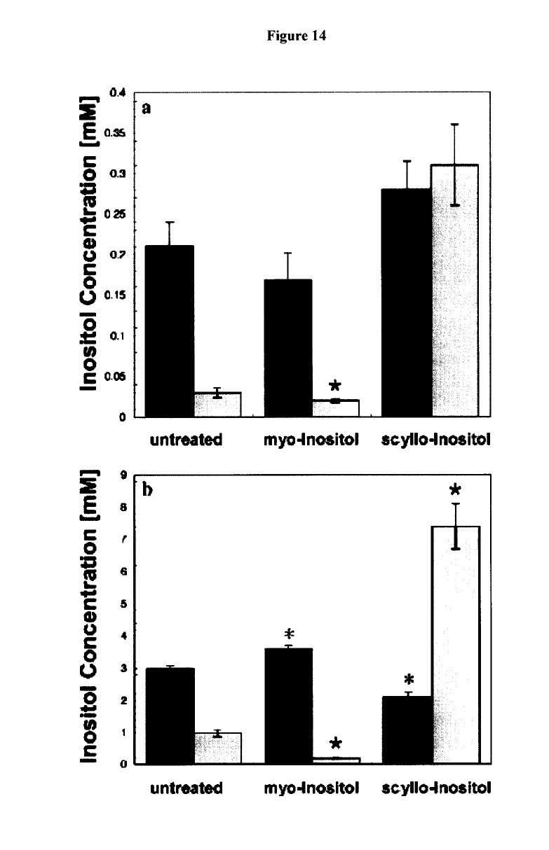

The elevation of CSF and brain inositol levels after myo or scyllo-inositol treatment was examined. At least 5 μl of mouse CSF was routinely isolated from the fourth ventricle before death. The CSF and brain levels of myo and scyllo-inositol in untreated animals were comparable to those reported previously in the literature (FIG. 14). myo-Inositol concentration is reported between 2 and 15 mM in the brain and between 0.08 and 0.3 mM in the CSF [1], which is comparable to the 3 mM in the brain and 0.21 mM in the CSF that was observed. scyllo-Inositol concentration is between 8 and 20% of myo-inositol levels [8, 9], which correlates well with the results. Furthermore, the untreated non-Tg littermate myo and scyllo-inositol concentrations were not different from the untreated TgCRND8 mice (P=0.96), suggesting that disease pathology, such as cerebrovascular amyloid, does not alter inositol levels.

To compare the effects of inositol administration on myo/scyllo-inositol equilibrium within the brain, the mice were administered with myo or scyllo-inositol ad libitum in drinking water at a concentration of 10 mg/ml. myo-Inositol and scyllo-inositol treatment did not significantly alter CSF myo-inositol concentration in comparison to the untreated controls (P=0.35 and 0.7, respectively; FIG. 14a). However, scyllo-inositol treatment caused a 16-fold increase in scyllo-inositol within the CSF (F_{2,25}=69.61; P<0.001; FIG. 13a). The increase in scyllo-inositol detected in CSF represents changes in the equilibrium between direct transport from the plasma and efflux from the brain tissue.

Whole brain homogenates were used to evaluate the inositol concentration in treated mice. The myo-inositol ad libitum treatment showed a 1.2-fold increase in brain myo inositol levels over the untreated controls (F_{2,22}=36.01; P<0.001) and a 5.4-fold decrease in scyllo-inositol levels (F_{2,23}=247.57; P=0.008; FIG. 14b). The concomitant decrease in scyllo-inositol after myo-inositol treatment may represent a shift in the inositol equilibrium towards degradation to stabilize brain homeostasis. The scyllo-inositol ad libitum treatment showed a 7.6-fold increase in scyllo-inositol over the control (F_{2,23}=247.6; P<0.001) and a 0.7-fold decrease in myo-inositol in the brain (P<0.001; FIG. 13b). These results suggest that sustained increases in inositol intake are reflected in a subsequent increase in the brain, with scyllo-inositol demonstrating the most significant change. The smaller changes in myo inositol may reflect the tight control necessary to maintain osmolarity and signal transduction pathways within the CNS.

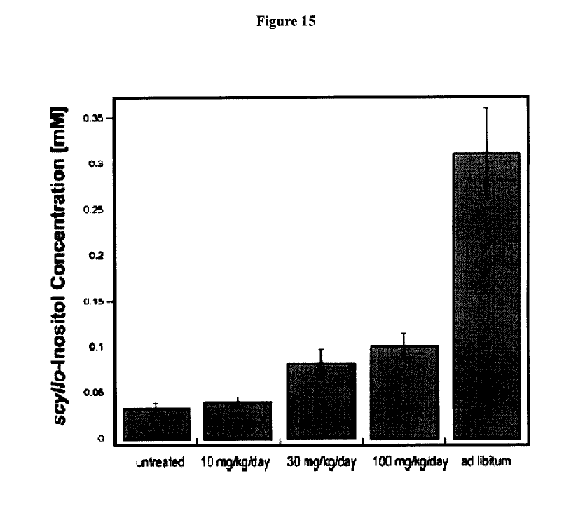

The ad libitum scyllo-inositol administration was compared to a once-daily 10-100 mg/kg/day treatment for 1 month to determine whether dosing once a day was sufficient to increase CNS scyllo-inositol levels. Approximately 8 h after the final treatment, the mice were killed for analyses of CSF inositol levels. The CSF analysis showed a non-significant dose-dependent increase in scyllo-inositol in comparison to the control mice (FIG. 15). The scyllo-inositol levels after ad libitum scyllo-inositol treatment were significantly higher than those after any of the single dose regimes (F_{4,17}=30.1; P<0.001). These results suggest that single-dose exposures of scyllo-inositol increase CSF levels but suggest that multiple dosing regimes may be advantageous for sustained high CNS scyllo-inositol concentrations.

myo-Inositol is an integral component of the phosphatidylinositol family of lipids as well as of signal transduction pathways. To rule out the incorporation of scyllo-inositol over myo-inositol into the phosphatidylinositol lipids, as a result of elevated scyllo-inositol brain concentrations, brain phospholipids were isolated to analyze head-group composition. scyllo-Inositol could not be detected in phosphatidylinositol lipids isolated from the brains of the control and scyllo-inositol-treated mice. scyllo-Inositol has an elution time of 18.2 min, and a point by point examination of the signal between 17 and 19 min failed to identify scyllo-inositol. Although minor concentrations of scyllo-inositol in the brain samples cannot be ruled out, the lower limit of detection is 0.25 ng/ml. These results suggest that scyllo-inositol does not substitute for myo-inositol when present at elevated concentrations within the CNS.

### 2. Discussion

The endogenous levels of scyllo-inositol within the brain have been reported for many species [8-11, 24]. Rat, rabbit, and bovine brain contains an epimerase that converts myo-inositol to scyllo-inositol [10, 12, 24]. Furthermore, myo-inositol administration in women demonstrated a 1% conversion to scyllo-inositol within the plasma [13], and the myo-inositol treatment in rats showed a 0.06% conversion to scyllo-inositol with a preferential 60% conversion to chiroinositol [14]. scyllo-Inositol may enter the brain using SMIT-1, which is known to transport both myo and scyllo-inositol with a preference for myo-inositol in vitro [15] and is expressed at the choroid plexus, a blood—CSF barrier [16]. This study confirms that myo- and scyllo-inositol cross from the periphery into the CNS.

It was previously shown that the treatment of TgCRND8 mice with scyllo-inositol ad libitum resulted in improvements in cognition and a decrease in toxic soluble Aβ species within the CNS. Dose-dependent effects on cognitive and pathological outcome measures were further shown. The current results after scyllo-inositol treatment shows a sustained inhibition of plaque formation and reduction in plaque burden in mice at the equivalent Aβ burden to humans at end-stage AD. In this study, changes in plaque size distribution between treated and untreated animals were examined alongside changes in the percent brain area covered in plaques to address the mechanism of scyllo-inositol interaction with and inhibition of plaque formation and expansion. This comparison demonstrated an important observation that scyllo-inositol was effectively and indiscriminately inhibiting the growth of plaques of all sizes. Thus, scyllo-inositol administration inhibits plaque growth by structurally interacting with both plaques and Aβ subunits. There are a number of hypotheses that can be proposed for scyllo-inositol inhibition of plaque growth. In vitro data showed the inhibition of Aβ42 fiber formation and stabilization of a small Aβ conformer that was non-toxic to nerve-growth factor-differentiated PC-12 cells [17]. In vivo data showed a decrease in soluble high molecular weight oligomers with a concomitant increase in Aβ monomers and trimers. Two mutually non-exclusive mechanisms are proposed: firstly, that scyllo-inositol can inhibit plaque growth by intercalating into the β-structure of growing aggregates and fibers, such that the growing face of the fiber is not conducive to further addition of Aβ peptides, or secondly, that scyllo-inositol may “cap-off” the growing edges of the Aβ aggregates to inhibit further assembly. Based on the additional observation that scyllo-inositol treatment results in a decrease in insoluble Aβ40 and Aβ42 without a concomitant increase in soluble Aβ levels or plasma levels, a further consequence of scyllo-inositol treatment is the acceleration of degradation and clearance from the brain.

Based on the GC/MS findings, ad libitum, but not single-dose scyllo-inositol treatment, resulted in a significant elevation in brain scyllo-inositol levels. These findings suggest that scyllo-inositol shows high CNS bioavailability, an important concern in designing a treatment for AD. If it is assumed that an elevation in brain scyllo-inositol levels is necessary for the efficacy of scyllo-inositol as an AD treatment strategy in humans, then a multiple dosing regiment might be preferable to a single daily dose, as it will cause a more sustained elevation in brain scyllo-inositol levels. In support of this suggestion, the dose finding studies demonstrated that at equivalent doses, twice-daily oral gavage administration of scyllo-inositol was more effective than one daily dose at reversing cognitive deficits in the TgCRND8 mice. Myo-inositol was not shown to be effective for the treatment of TgCRND8 mice despite in vitro efficacy [17]. This may be due to the interconversion of myo inositol into scyllo- and chiroinositol in the plasma, and the low fold increase in brain myo-inositol levels, or may suggest a tighter regulation of myo-inositol within the CNS, as would be expected for a compound involved in osmolarity and signal transduction pathways. In contrast, scyllo-inositol is not known to participate in signaling pathways in the brain, and no evidence of adverse effects were seen in the mice.

Scyllo-inositol can be detected in the human brain in vivo using proton nuclear magnetic resonance spectroscopy [9, 18] at levels similar to those reported for post-mortem tissue. Previous studies have suggested that scyllo-inositol is elevated in some brain tumors and disease states and was attributed to an astrogliotic response [9, 19, 20]. In contrast, high levels of scyllo-inositol were observed in a healthy subject with a normal neurological status [8]. No adverse side effects were observed in this individual despite a myo to scyllo-inositol ratio of 5:1 rather than the more typical 12:1 ratio [8]. This study supports the suggestion that a sustained elevated scyllo-inositol concentration is not detrimental, as the treated animals showed no cognitive or pathological side effects.

An additional aim of this study was to determine whether an increase in brain scyllo-inositol would result in a preferential incorporation of scyllo- over myo-inositol into phosphatidylinositol lipids, and thereby predict secondary effects on signal transduction pathways. To date, only ciliate and barley seeds have been shown to incorporate scyllo-inositol into lipids, both as a result of elevated exposure and endogenous conditions; in contrast, studies in mammals have failed to detect endogenous scyllo-inositol incorporation into lipids [21-23]. This study confirms that scyllo-inositol is not incorporated into the phosphatidylinositol family of lipids after ad libitum treatment with scyllo-inositol. In conclusion, the results demonstrate that orally administered scyllo-inositol increases both CSF and brain scyllo-inositol levels that result in disease-modifying effects in a mouse model of AD.

### Example 7

The ability of inositol isomers to cross the blood-brain barrier and subsequently increase the steady-state brain inositol levels was investigated.

### Objectives:

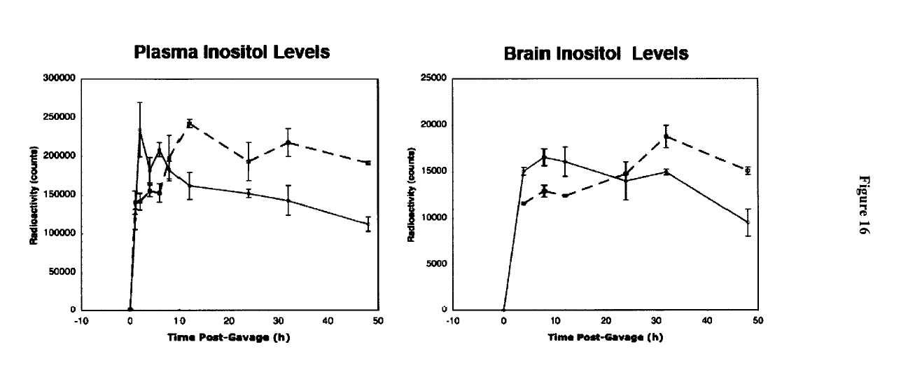

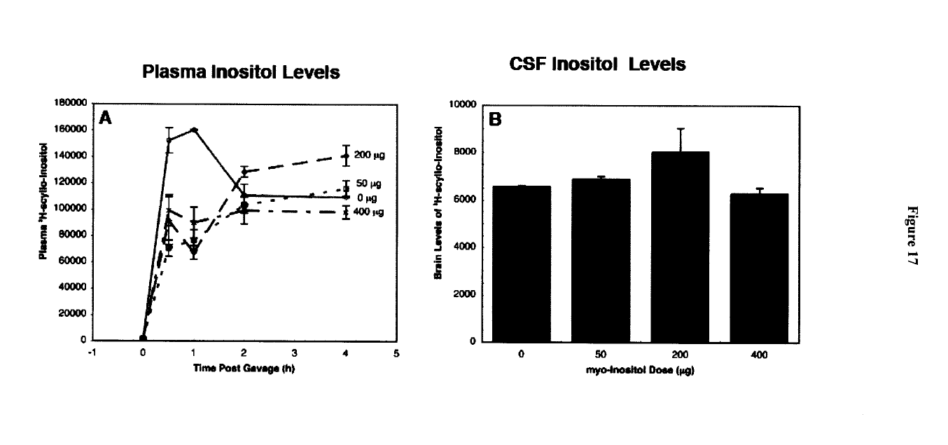

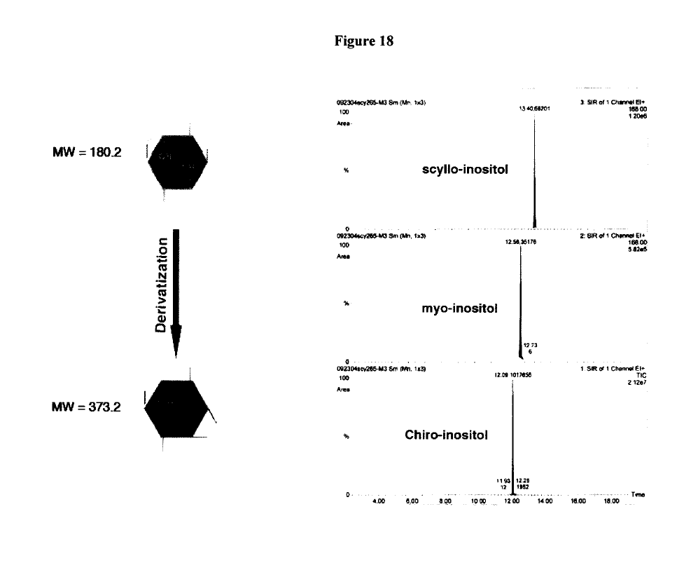

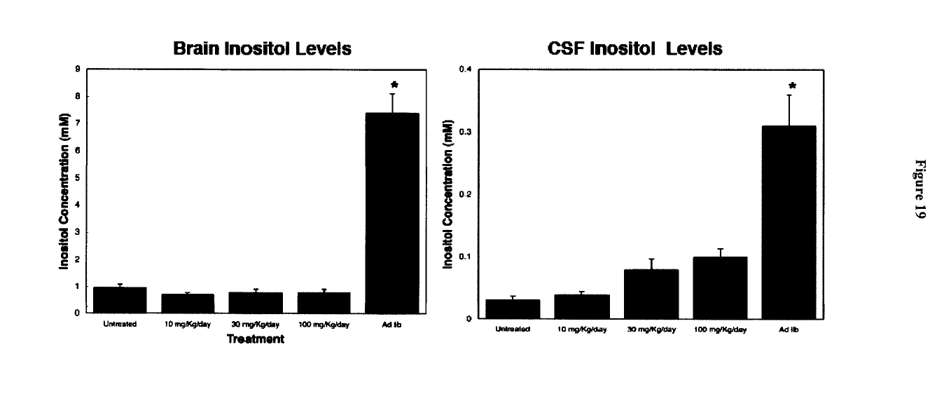

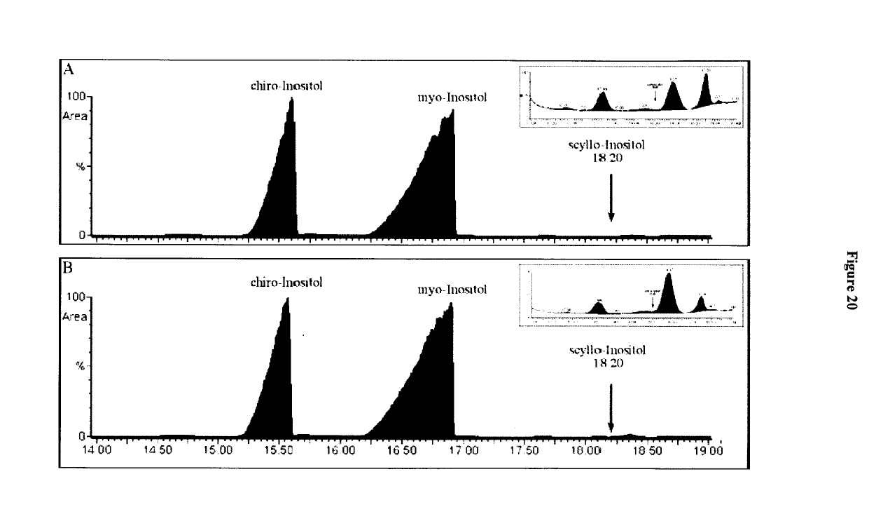

1. To determine whether the concentration of inositol is altered in the brain after peripheral administration. The peripheral administration of inositol stereoisomers may result in an increased concentration of these compounds in the cerebral spinal fluid and the brain.
2. Determine whether this results in increased incorporation into phosphoinositides or the phosphatidylinositol family of lipids.
   Methods: To test this hypothesis, gas chromatography/mass spectrometry was used to measure inositol levels in the brain and the cerebral spinal fluid after oral dose administration. To increase the volatility and thermal stability of these compounds and to allow for proper peak separation the hydroxyl groups of inositol were derivatized with acetyl groups (adapted from Shetty H U et al, 1995, Anal Biochem 224:279-285). Improved quantification of inositol was found by monitoring mass ion 168. Inositol levels in phosphatidylinositol lipids and phosphoinositides were quantified by inositol isolation using thin layer chromatography and acid hydrolysis (Kersting et al, 2003, J Eukaryot Microbiol) followed by gas chromatography/mass spectrometry.
   Results: The results are shown in FIGS. 14, and 16 to 20. Peripherally administered inositols showed a rapid increase in plasma inositol levels as monitored by radioactively labeled inositol compounds. Continuous administration of some inositols increased concentrations in both brain and CSF. The increase in inositol levels did not alter phosphoinositide levels nor incorporation of peripherally administered inositol into phosphatidylinositol family of lipids. Dose response increases were observed for the most effective inositol isomers.
   Conclusions: These results show that peripheral administration dose dependently increases brain steady state inositol levels.

### Example 8

### A Phase 1 Single Ascending Dose, Double-Blind, Randomized, Placebo-Controlled Study to Evaluate the Safety, Tolerability and Pharmacokinetic Profile of Oral Doses of AZD-103 in Healthy Male Volunteers

### Objectives:

1. To assess the safety and tolerability of single ascending doses of AZD-103 when administered orally to healthy male volunteers.
2. To assess the pharmacokinetic (PK) profile of single ascending doses of AZD-103 when administered orally to healthy male volunteers.

### Study Design:

AZD-103 (scyllo-inositol) was administered to six sequential cohorts of healthy male volunteers. Each cohort received a different dose level of AZD-103. Dose levels were escalated between cohorts, such that the first cohort received the lowest dose level, the middle cohorts received intermediate dose levels, and the final cohort received the highest proposed dose level. Dose escalation was dependent on a review of safety data from the previously dosed cohort.

Forty eight (48) healthy males were randomly assigned in a 3:1 fashion to receive either AZD-103 or placebo (36 subjects receive active drug and 12 subjects receive placebo). Six escalating doses of AZD-103 were evaluated in six different subject cohorts so that each subject received only one dose of study drug. Each cohort included 8 subjects, with 6 subjects randomized to AZD-103 and 2 subjects randomized to placebo. Dosing started with the lowest dose and escalated to the highest dose. The starting dose is 500 mg followed by planned doses of 1000 mg, 2000 mg, 3500 mg, 5000 mg and 7000 mg.

Subjects received their single dose of study drug on the study day 1 (Day 1). Throughout the day on Day 1, subjects were monitored for safety and PK samples were drawn. On Day 2, subjects were monitored for safety and PK samples were drawn. Subjects were discharged on Day 3 after final safety monitoring and PK sampling was complete, approximately 48 hours after their single dose of study drug.

Upon completion of Cohort 1, a comprehensive safety review of subject safety data will be undertaken and dosing of Cohort 2 will only proceed upon a satisfactory joint safety review by the Principal Investigator and Sponsor. This procedure will also be followed before dosing of subsequent cohorts proceeds.

Study Population: 48 healthy male volunteers
Study Treatments Subjects will receive either placebo or AZD-103 capsules

### Safety Endpoints Incidence of AEs and SAEs

Laboratory safety tests (hematology, biochemistry, urinalysis)
Vital signs
ECG

### PK Endpoints

Plasma concentrations of AZD-103 (AUC, Cₘₐₓ, tₘₐₓ, t_{1/2}, Kₑₗ)

### Results:

The mean peak exposure was 5.82 μg/mL in Cohort 1 (500 mg), 16.99 μg/mL in Cohort 2 (1000 mg), 33.11 μg/mL in Cohort 3 (2000 mg), 74.62 μg/mL in Cohort 4 (3500 mg), 109.5 μg/mL in Cohort 5 (5000 mg) and 154.56 μg/mL in Cohort 6 (7000 mg). Looking at the individual profiles of subjects in the six Cohorts, it was observed that mean AZD-103 Tₘₐₓ (±SD) were 4.00±1.55 h (500 mg), 3.25±1.47 h (1000 mg), 2.75±0.61 h (2000 mg), 2.75±0.61 h (3500 mg), 2.42±0.66 h (5000 mg) and 2.42±0.67 h (7000 mg) following oral administration.

AZD-103 elimination appears bi-phasic with a mean terminal elimination half-life, evaluated in Cohort 6 (7000 mg), of 18.37 hours.

The mean area under the curve from time zero to the last predicted concentration was 29.86 μg·h/mL in Cohort 1 (500 mg), 111.8 μg·h/mL in Cohort 2 (1000 mg), 194.21 μg·h/mL in Cohort 3 (2000 mg), 440.63 μg·h/mL in Cohort 4 (3500 mg), 490.84 μg·h/mL in Cohort 5 (5000 mg) and 859.88 μg·h/mL in Cohort 6 (7000 mg).

For Cohort 6 (7000 mg), the mean area under the curve from time zero to infinity was 885.35 μg·h/mL, the mean apparent volume of distribution was 213.98 L and the mean total body clearance was 8.08 L/h.

Dose-proportionality was evaluated by normalizing the pharmacokinetic parameter (Cₘₐₓ and AUC₀₋ₜ) by the dose. Mean dose normalized Cₘₐₓ values were 0.01, 0.02, 0.02, 0.02, 0.02 and 0.02 (μg/mL)/mg for the 500 mg, 1000, 2000, 3500, 5000 and 7000 mg doses, respectively. Mean dose normalized AUC₀₋ₜ values were 0.06, 0.112, 0.1, 0.13, 0.10 and 0.12 (μg/mL)/mg for the 500 mg, 1000, 2000, 3500, 5000 and 7000 mg doses, respectively. These findings are consistent with dose-proportionality of AZD-103 in the 1000 to 7000 mg oral dose range.

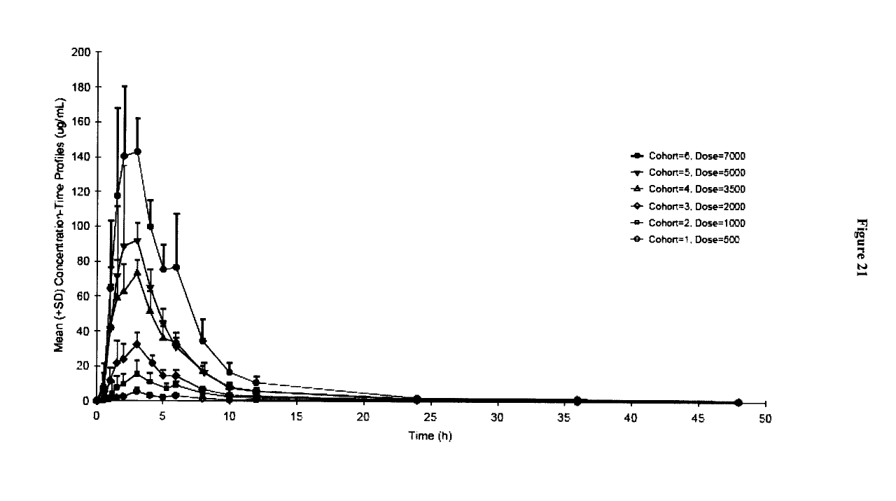

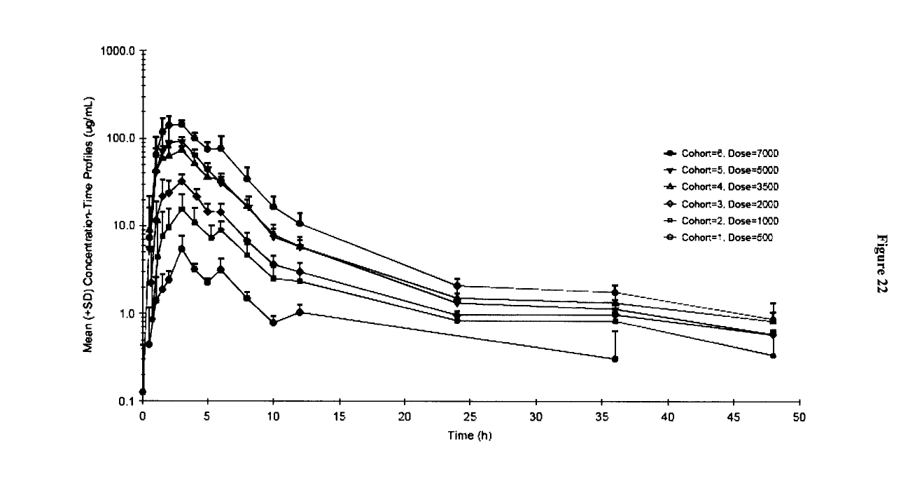

As shown in Table 11, the PK parameters appear to increase proportionally with increasing dose up to and including 3500 mg. The increase in Cₘₐₓ and AUC is less than proportional at the 5000 mg dosage; this suggests saturation in the absorption process. The mean elimination half-life (t_{1/2}) ranges from 13 24 hrs; however, the plasma concentration vs. time profiles (FIGS. 21 and 22) suggest that approximately 80-85% of the AUC is accounted for in the first 12 hrs.

Having illustrated and described the principles of the invention in specific preferred embodiments, it should be appreciated to those skilled in the art that the invention can be modified in arrangement and detail without departure from such principles. We claim all modifications coming within the scope of the following claims.

All publications, patents and patent applications referred to herein are incorporated by reference in their entirety to the same extent as if each individual publication, patent or patent application was specifically and individually indicated to be incorporated by reference in its entirety.

TABLE 1
Preclinical pharmacokinetic studies

| | | | | Doses | |
|---|---|---|---|---|---|
| | | Treatment | Total daily dose | administered | PK |
| Species | n= | duration | (mg/kg/day) | (mg/kg) | timings |
| Rat | 36 | 28 days | 30, 100, 300; po | 15, 50, 150; po | Days 1, 28 |
| Dog | 18 | 14 days | 30, 100, 300; po | 15, 50, 150; po | Days 1, 14 |
| Dog | 5 | Single | 20, 80, 240; po | 20, 80, 240; po | Single |
| | | dose | 80, 240; iv | 80, 240; iv | dose |

TABLE 2
Mean pharmacokinetic parameters (±SD)
of single oral doses of AZD103 in rats

| | 15 mg/kg | 50 mg/kg | 150 mg/kg |
|---|---|---|---|
| n | 6 | 6 | 6 |
| Cₘₐₓ (μg mL⁻¹)* | 13.2 ± 2.0 | 45.3 ± 7.1 | 130.8 ± 54.8 |
| tₘₐₓ (h) | 1.0 | 1.5 ± 0.5 | 2.2 ± 1.5 |
| AUC₀₋ₜ (μg | 39.7 ± 11.3 | 162.8 ± 29.0 | 537.4 ± 153.8 |
| h⁻¹ mL⁻¹)* | | | |
| AUC_{0-∞} (μg | 41.9 ± 11.9 | 169.1 ± 31.8 | 551.6 ± 154.4 |
| h⁻¹ mL⁻¹)* | | | |
| kₑₗ (h⁻¹) | 0.27 ± 0.07 | 0.29 ± 0.02 | 0.33 ± 0.03 |
| t_{1/2} (h)* | 2.7 ± 0.6 | 2.4 ± 0.2 | 2.1 ± 0.2 |
| Cl/F (mL h⁻¹) | 767.6 ± 220.5 | 608.5 ± 109.0 | 572.9 ± 127.0 |
| Vd_{SS} (mL) | 2,821 ± 774.3 | 2,134 ± 169.6 | 2,239 ± 719.7 |
| MRT (h) | 3.7 ± 0.5 | 3.6 ± 0.6 | 3.9 ± 0.7 |

*P < 0.05 (Analysis of Variance)

TABLE 3
Mean pharmacokinetic parameters (±SD)
of single oral doses of AZD103 in dogs

| | 15 mg/kg | 50 mg/kg | 150 mg/kg |
|---|---|---|---|
| N | 6 | 6 | 6 |
| Cₘₐₓ (μg mL⁻¹)* | 10.0 ± 2.5 | 47.4 ± 12.7 | 124.0 ± 22.7 |
| tₘₐₓ (h)* | 1.7 ± 0.5 | 1.2 ± 0.4 | 2.2 ± 0.4 |
| AUC₀₋ₜ (μg | 22.6 ± 4.5 | 113.7 ± 22.9 | 450.1 ± 118.0 |
| h mL⁻¹)* | | | |
| AUC_{0-∞} (μg | 23.8 ± 4.5 | 115.8 ± 22.8 | 455.1 ± 121.3 |
| h mL⁻¹)* | | | |
| kₑₗ (h⁻¹) | 0.4 ± 0.2 | 0.3 ± 0.04 | 0.4 ± 0.05 |
| t_{1/2} (h) | 2.0 ± 0.8 | 2.1 ± 0.3 | 1.7 ± 0.2 |
| Cl/F (mL kg⁻¹ h⁻¹)* | 182.5 ± 54.8 | 124.3 ± 36.7 | 93.7 ± 26.7 |
| Vd_{SS} (mL kg⁻¹)* | 632.5 ± 237.1 | 365.9 ± 121.9 | 307.4 ± 74.4 |
| MRT (h) | 3.5 ± 0.8 | 2.9 ± 0.3 | 3.3 ± 0.3 |

*P < 0.05 (Analysis of Variance)

TABLE 4
Mean pharmacokinetic parameters (±SD) of single oral and intravenous doses of AZD103 in dogs

| | Oral | | | Intravenous | |
|---|---|---|---|---|---|
| | 20 mg/kg | 80 mg/kg | 240 mg/kg | 80 mg/kg | 240 mg/kg |
| N | 5 | 5 | 4 | 4 | 4 |
| Cₘₐₓ (μg mL⁻¹) | 5.9 ± 3.1 | 69.0 ± 23.5 | 89.5 ± 27.8 | ND | ND |
| Cp₀* (μg mL⁻¹) | ND | ND | | 87.2 ± 13.2 | 127.9 ± 19.1 |
| tₘₐₓ (h) | 1.5 ± 0.6 | 2.0 ± 1.2 | 2.0 ± 1.4 | ND | ND |
| AUC₀₋ₜ (μg h mL⁻¹) | 42.1 ± 8.4 | 243 ± 54 | 477 ± 94 | 167 ± 7 | 244 ± 24 |
| AUC_{0-∞} (μg h mL⁻¹) | 51.7 ± 15.7 | 246 ± 53 | 489 ± 99 | 173 ± 9 | 248 ± 25 |
| kₑₗ (h⁻¹) | 0.2 ± 0.2 | 0.4 ± 0.1 | 0.4 ± 0.1 | 0.21 ± 0.02 | 0.30 ± 0.02 |
| t_{1/2} (h) | 5.0 ± 3.9 | 2.0 ± 0.3 | 2.0 ± 0.4 | 3.3 ± 0.3 | 2.3 ± 0.2 |
| Cl/F (mL h⁻¹) | 418 ± 137 | 338 ± 76 | 507 ± 108 | 464 ± 24 | 975 ± 96 |
| Vd_{SS} (mL) | 3,765 ± 1246 | 1,419 ± 603 | 2,638 ± 639 | 2,105 ± 462.5 | 3,222 ± 482.0 |
| MRT (h) | 10.1 ± 5.9 | 4.1 ± 0.8 | 5.2 ± 0.2 | 4.6 ± 1.2 | 3.3 ± 0.5 |

ND: not determined

TABLE 5
Mean pharmacokinetic parameters (±SD) of AZD103
after 28 days dosing, twice daily, in rats

| | 15 mg/kg | 50 mg/kg | 150 mg/kg |
|---|---|---|---|
| N | 6 | 6 | 6 |
| Cₘₐₓ (μg mL⁻¹)* | 28.5 ± 4.9 | 59.4 ± 8.4 | 170.8 ± 79.9 |
| tₘₐₓ (h) | 1.3 ± 0.5 | 1.2 ± 0.4 | 1.2 ± 0.4 |
| AUC₀₋ₜ (μg | 161.0 ± 57.7 | 271.1 ± 70.7 | 573.0 ± 127.7 |
| h⁻¹ mL⁻¹)* | | | |
| AUC_{0-∞} (μg | 194.8 ± 71.7 | 297.8 ± 82.2 | 614.7 ± 128.8 |
| h⁻¹ mL⁻¹)* | | | |
| kₑₗ (h⁻¹)* | 0.16 ± 0.03 | 0.21 ± 0.03 | 0.22 ± 0.02 |
| t_{1/2} (h)* | 4.4 ± 0.6 | 3.3 ± 0.5 | 3.1 ± 0.3 |
| Cl/F (mL h⁻¹)* | 174.2 ± 66.6 | 357.0 ± 93.3 | 504.4 ± 94.8 |
| Vd_{SS} (mL)* | 1,135 ± 384.4 | 1,728 ± 430.9 | 2,344 ± 779.7 |
| MRT (h)* | 6.7 ± 0.6 | 4.9 ± 0.8 | 4.6 ± 0.8 |

*P < 0.05 (Analysis of Variance)

TABLE 6
Mean pharmacokinetic parameters (±SD) of AZD103
after 14 days dosing, twice daily, in dogs

| | 15 mg/kg | 50 mg/kg | 150 mg/kg |
|---|---|---|---|
| N | 6 | 6 | 6 |
| Cₘₐₓ (μg mL⁻¹)* | 16.4 ± 4.5 | 59.4 ± 13.0 | 121.3 ± 26.7 |
| tₘₐₓ (h)* | 1.7 ± 0.5 | 1.1 ± 0.5 | 1.8 ± 0.4 |
| AUC₀₋ₜ (μg | 75.2 ± 9.4 | 231.8 ± 50.2 | 522.7 ± 122.6 |
| h mL⁻¹)* | | | |
| AUC_{0-∞} (μg | 95.16 ± 18.6 | 262.3 ± 52.4 | 557.4 ± 133.1 |
| h mL⁻¹)* | | | |
| kₑₗ (h⁻¹) | 0.2 ± 0.08 | 0.2 ± 0.1 | 0.3 ± 0.04 |
| t_{1/2} (h) | 4.7 ± 2.0 | 3.8 ± 1.2 | 2.9 ± 0.6 |
| Cl/F (mL kg⁻¹ h⁻¹)* | 41.9 ± 9.4 | 51.6 ± 14.7 | 72.1 ± 19.1 |
| Vd_{SS} (mL kg⁻¹) | 301.7 ± 41.9 | 272.6 ± 121.7 | 332.1 ± 88.1 |
| MRT (h)* | 7.4 ± 1.5 | 5.3 ± 1.2 | 4.6 ± 0.4 |

*P < 0.05 (Analysis of Variance)

TABLE 7
Mean pharmacokinetic parameters (±SD) in plasma and
CSF following of single oral doses of AZD103 in dogs

| | | |
|---|---|---|
| 20 mg/kg | 80 mg/kg | 240 mg/kg |

PLASMA LEVELS

| | | | |
|---|---|---|---|
| N | 4 | 4 | 3 |
| Cₘₐₓ (μg mL⁻¹) | 6.1 ± 3.6 | 68.8 ± 27.2 | 101.4 ± 17.8 |
| tₘₐₓ (h) | 2.8 ± 2.2 | 2.3 ± 1.3 | 2.3 ± 1.5 |
| AUC₀₋ₜ (μg | 42.6 ± 9.6 | 249.8 ± 59.3 | 514.5 ± 0.3 |
| h mL⁻¹) | | | |
| AUC_{0-∞} (μg | 52.7 ± 17.9 | 254.7 ± 58.6 | 527.8 ± 74.6 |
| h mL⁻¹) | | | |

CSF

| | | | |
|---|---|---|---|
| n | 4 | 4 | 3 |
| Cₘₐₓ (μg mL⁻¹) | 2.3 ± 1.4 | 18.7 ± 1.0 | 67.4 ± 28.6 |
| tₘₐₓ (h) | 4.0 ± 1.7 | 2.8 ± 1.5 | 3.0 ± 1.7 |
| AUC₀₋ₜ (μg | 20.6 ± 6.6 | 98.0 ± 13.2 | 384.4 ± 203.5 |
| h mL⁻¹) | | | |
| AUC_{0-∞} (μg | 38.0 ± 22.8 | 103.3 ± 13.1 | 400.8 ± 203.6 |
| h mL⁻¹) | | | |

Plasma and CSF mean values are obtained using data from exactly the same animals. For this reason there are small differences between the plasma statistics listed here, and the statistics listed in Table 5, which were based on all animals receiving oral administration.

TABLE 8
Mean levels of AZD-103 in CSF and plasma 24 and
12 hours, respectively, after 14 days dosing

| Dose (mg/kg) | CSF level at 24 h (μg/ml) | Plasma level at 12 h (μg/ml) |
|---|---|---|
| 30 | 4.0 | 3.6 |
| 100 | 7.5 | 5.8 |
| 300 | 10.9 | 8.2 |

TABLE 9
AZD-103 reduces Aβ and plaque accumulation
when dosed therapeutically (5-6 months age).

| | Aβ40 | | Aβ40 | | Total | Mean |
|---|---|---|---|---|---|---|
| | (ng/gm wet brain ± sem) | | (ng/gm wet brain ± sem) | | Plaque | Plaque |
| | Soluble | Insoluble | Soluble | Insoluble | Area (μm²) | Size (μm²) |
| Control | 204 ± 4 | 4965 ± 457 | 426 ± 14 | 14503 ± 1071 | 486002 ± 16156 | 401 ± 14 |
| AZD-103 | 178 ± 11 | 3527 ± 241* | 374 ± 23 | 11115 ± 647* | 420027 ± 14986* | 336 ± 6* |

ANOVA with Fisher's PLSD,
*p < 0.05

TABLE 10
Scyllo-inositol treatment decreases Aβ40 and Aβ42 levels

| | Brain Aβ40 | | Brain Aβ42 | | Plasma Aβ levels | |
|---|---|---|---|---|---|---|
| | (ng/gm wet brain ± SEM) | | (ng/gm wet brain ± SEM) | | (pg/ml) | |
| 2 month treatment | Soluble | Insoluble | Soluble | Insoluble | Aβ40 | Aβ42 |
| Control | 487 ± 14 | 6924 ± 287 | 764 ± 51 | 25827 ± 1238 | 5212 ± 219 | 3455 ± 331 |
| scyllo-Inositol | 395 ± 60 | 5703 ± 612* | 688 ± 28 | 20818 ± 1404* | 4507 ± 207 | 3035 ± 236 |

ANOVA with Fisher's PLSD,
*p < 0.05.

TABLE 11
Mean (±SD) Pharmacokinetic Parameter Estimatin of ELND005 in Healthy Adult Men after Single Oral Dosages

| | Parameters | | | | | | |
|---|---|---|---|---|---|---|---|
| Dose | Cmax | Tmax | AUC₀₋ₜ | AUC_{0-INF} | T_{1/2} | CL/F | MRT |
| mg | μmL | h | μ · h/mL | μ · h/mL | h | L/h | h |
| 500 | 5.825 ± 1.83 | 4.00 ± 1.55 | 29.86 ± 7.11 | 42.51 ± 22.13 | 13.33 ± 16.72 | 13.67 ± 5.49 | 19.09 ± 21.81 |
| 1000 | 16.995 ± 5.295 | 3.25 ± 1.47 | 111.37 ± 38.64 | 129.50 ± 44.37 | 22.48 ± 5.75 | 8.51 ± 2.78 | 19.71 ± 3.17 |
| 2000 | 33.11 ± 7.573 | 2.75 ± 0.61 | 194.05 ± 36.08 | 214.97 ± 41.88 | 23.88 ± 7.24 | 9.59 ± 1.80 | 16.30 ± 3.99 |
| 3500^{a} | 74.63 ± 9.24 | 2.75 ± 0.61 | 440.63 ± 71.43 | 467.12 ± 77.69 | 20.30 ± 3.09 | 7.71 ± 1.59 | 10.98 ± 1.43 |
| 5000^{a} | 109.50 ± 26.94 | 2.42 ± 0.67 | 490.65 ± 73.19 | 506.75 ± 71.95 | 16.91 ± 7.10 | 10.04 ± 1.49 | 8.23 ± 2.0 |
| 7000 | 154.557 ± 20.411 | 2.42 ± .067 | 859.88 ± 148.25 | 885.35 ± 150.68 | 18.38 ± 4.64 | 8.08 ± 1.22 | |

### CITATIONS FOR PUBLICATIONS REFERRED TO IN EXAMPLE 6

1. Fisher S K, Novak J E, Agranoff B W (2002) Inositol and higher inositol phosphates in neural tissues: homeostasis, metabolism and functional significance. J Neurosci 82:736-754
2. Chishti M A, Yang D, Janus C et al (2001) Early-onset amyloid deposition and cognitive deficits in transgenic mice expressing a double mutant form of amyloid precursor protein 695. J Biol Chem 276:21562-21570
3. Shetty H U, Holloway H W, Rapoport S I (1995) Capillary gas chromatography combined with ion trap detection for quantitative profiling of polyols in cerebrospinal fluid and plasma. Anal Biochem 224:279-285
4. Kersting M C, Boyette M, Massey J H, Ryals P E (1993) Identification of the inositol isomers present in Tetrahymena. J Eukaryot Microbiol 50:164-168
5. Wang J, Dickson D W, Trojanowski J Q, Lee V M-Y (1999) The levels of soluble versus insoluble brain Aβ distinguish Alzheimer's disease from normal and pathological aging. Exp. Neurol. 158, 328-337
6. Naslund J, Haroutunian V, Mohs R et al (2000) Correlation between elevated levels of amyloid β-peptide in the brain and cognitive decline. JAMA 283, 1571-1577
7. Li R, Lindholm K, Yang L-B et al (2004) Amyloid β peptide load is correlated with increased β-secretase activity in sporadic Alzheimer's disease patients. Proc. Natl. Acad. Sci. USA 101, 3632-3637
8. Seaquist E R, Gruetter R (1998) Identification of a high concentration of scyllo-inositol in the brain of a healthy human subject using 1H- and 13C-NMR. MRM 39:313-316
9. Michaelis T, Helms G, Merboldt K-D, Hanicke W, Bruhn H, Frahm J (1993) Identification of scyllo-inositol in proton NMR spectra of human brain in vivo. NMR Biomed 6:105-109
10. Sherman W R, Stewart M A, Simpson P C, Goodwin S L (1968) The identification of myo-inosose-2 and scyllo-inositol in mammalian tissues. Biochemistry 7:819-824
11. Spector R (1978) The transport and metabolism of scyllo-inositol in the CNS. J Neurochem 31:1113-1115
12. Hipps P P, Holland W H, Sherman W R (1977) Interconversion of myo- and scyllo-inositol with simultaneous formation of neo-inositol by an NADP dependent epimerase from bovine brain. Biochem Biophys Res Commun 77:340-346
13. Groenen P M W, Merkus H M W M, Sweep F C G J, Wevers R A, Janssen F S M, Steegers-Theunissen RPM (2003) Kinetics of myo-inositol loading in women of reproductive age. Ann Clin Biochem 40:79-85
14. Pak Y, Huang L C, Lilley K J, Lamer J (1992) In vivo conversion of [3H]myoinositol to [3H]chiroinositol in rat tissues. J Biol Chem 267:16904-16910
15. Hager K, Hazama A, Kwon H M, Loo D D, Handler J S, Wright E M (1995) Kinetics and specificity of the renal Na⁺/myo-inositol cotransporter expressed in Xenopus oocytes. J Membr Biol 143:103-113
16. Inoue K, Shimada S, Minami Y, Morimura H, Miyai A, Yamauchi A, Tohyama M (1996) Cellular localization of Na+/MYO-inositol co-transporter mRNA in the rat brain. NeuroReport 7:1195-1198
17. McLaurin J, Goloumb R, Jurewicz A, Antel J P, Fraser P E (2000) Inositol stereoisomers stabilize an oligomeric aggregate of Alzheimer amyloid beta peptide and inhibit abeta-induced toxicity. J Biol Chem 275:18495-18502
18. Kaiser L G, Schuff N, Cashdollar N, Weiner M W (2005) Scyllo-inositol in normal aging human brain: 1H magnetic resonance spectroscopy study at 4 Tesla. NMR Biomed 18:51-55
19. Frahm J, Bruhn H, Hanicke W, Merboldt K D, Mursch K, Markakis E (1991) Localized proton NMR spectroscopy of brain tumors using short-echo time STEAM sequences. J Comput Assist Tomogr 15:915-922
20. Meyerhoff D, Fein G, Weiner M (1996) Elevated scyllo-inositol in adult human brain. In: Proc ISMRM, 4^{th} Scientific Meeting, New York, pp 954.
21. Ryals P E, Kersting M C (1999) Sodium-dependent uptake of [3H]scyllo-inositol by Tetrahymena: Incorporation into phosphatidylinositol, phosphatidylinositol-linked glycans, and polyphosphoinositols. Arch Biochem Biophys 366:261-266
22. Murthy P P N, Pliska-Matyshak G, Keranen L M, Lam P, Mueller H H, Bhuvarahamurthy N (1992) Evidence of two isomers of phosphatidylinositol in plant tissue. Plant Physiol 98:1498-1501
23. Kinnard R L, Narasimhan B, Pliska-Matyshak G, Murthy P P N (1995) Characterization of scyllo-inositol-containing phosphatidylinositol in plant cells. Biochem Biophys Res Comm 210:549-555
24. Sherman W R, Stewart M A, Kurien M M, Goodwin S L (1968) The measurement of myo-inositol, myo-inosose-2 and scyllo-inositol in mammalian tissues. Biochim Biophys Acta 158:197-205
25. Uldry M, Ibberson M, Horisberger J-D, Chatton J-Y, Riederer B M, Thorens B (2001) Identification of a mammalian H-myo-inositol symporter expressed predominantly in the brain. EMBO J. 20:4467-4477

What is claimed is:

1. A dosage form comprising an amount of a cyclohexane polyalcohol compound suitable for administration to a subject to provide a therapeutically effective concentration of the compound in plasma, brain and/or cerebral spinal fluid or to provide at least one therapeutic effect in the prevention, treatment, or control of symptoms of a disorder in protein folding and/or aggregation, and/or amyloid formation, deposition, accumulation, or persistence.

2. A dosage form according to claim 1 that maintains the compound within a therapeutically effective concentration in plasma.

3. A dosage form according to claim 2 wherein the plasma concentration of the compound is at least about 0.05 μM.

4. A dosage form according to any preceding claim wherein the plasma concentration of the compound is at least about 1 to 50 μM, 1 to 20 μM, 1 to 10 μM, 1 to 6 μA or 1 to 5 μM.

5. A dosage form according to claim 1 that maintains the compound within a therapeutically effective concentration in the cerebral spinal fluid (CSF).

6. A dosage form according to claim 5 wherein the CSF concentration of the compound is at least about 0.05 μM.

7. A dosage form according to claim 6 wherein the CSF concentration of the compound is at least about 1 to 50 μM, 1 to 20 μM, 1 to 10 μM, 1 to 6 μM or 1 to 5 μM.

8. A dosage form according to any preceding claim for once or twice a day administration comprising a dose of compound that provides an extent of absorption, as defined by an area under the curve (AUC) equivalent to those produced by three or more a day dosage forms of the compound.

9. A dosage form according to any preceding claim comprising a dose of compound that provides a minimum concentration of the compound, Cₘᵢₙ, that is not statistically significantly different from that obtained with a dosage form administered more than twice a day over a dosing period.

10. A dosage form according to any preceding claim comprising a dose of compound that provides a T_{1/2} of 1 to 35 hours.

11. A dosage form according to any preceding claim for twice daily administration that has a bioavailability, as measured by AUC, of at least 50%, 60%, 65%, 70%, 75%, 80%, 85%, or 90% of the bioavailability of a single daily dosage form.

12. A dosage form according to any preceding claim wherein the cyclohexane polyalcohol compound is in a dose that provides a stoichiometric relationship of cyclohexane polyalcohol to amyloid peptide of about 40:1, 35:1. 30:1, 25:1, 20:1 or 15:1, preferably 25:1.

13. A dosage form comprising an amount of a cyclohexane polyalcohol compound suitable for administration to a subject to provide a therapeutically effective concentration of the compound in plasma, brain and/or cerebral spinal fluid and a pharmaceutically acceptable carrier, diluent or excipient, wherein when the formulation is administered in a dose of 500, 1000, 2000, 3500, 5000 or 7000 mg of said cyclohexane polyalcohol, a mean plasma concentration profile is achieved having a mean AUC_{0-INF} in μ·h/mL of, respectively, 43±20%, 130±20%, 215±20%, 467±20%, 507±20% or 885±20%, and having a mean Cₘₐₓ in μmL of, respectively, 5.8±20%, 17±20%, 33±20%, 75±20%, 110±20% or 155±20%.

14. A dosage form according to claim 1 suitable for administration once a day or twice a day.

15. A dosage form according to any preceding claim which is suitable for oral administration.

16. A dosage form according to any preceding claim which is a sustained release dosage form.

17. A dosage form according to any preceding claim suitable for oral administration once a day or twice a day wherein the compound is present in an amount sufficient so that the formulation exhibits a favourable or improved in vitro dissolution profile.

18. A dosage form according to any preceding claim suitable for once a day administration.

19. A dosage form according to any preceding claim which provides a zero-order or near zero-order release profile.

20. A dosage form according to any preceding claim suitable for twice a day administration.

21. A dosage form comprising a cyclohexane polyalcohol compound comprising a first dose for administration at a first time point and a second dose for administration at a second time point over a dosing period, wherein the dosage form comprises an amount of compound sufficient to provide a beneficial pharmacokinetic profile and the concentration or peak concentration of compound in plasma, brain or CSF does not significantly vary during the dosing period.

22. A dosage form comprising a cyclohexane polyalcohol compound comprising a first dose for administration to a subject at a first time point and a second dose for administration to the subject at a second time point over a dosing period, wherein the dosage form comprises an amount of compound sufficient to provide a Cₘᵢₙ in plasma, brain or CSF after the second time point greater than the Cₘᵢₙ after the first time point.

23. A dosage form according to any preceding claim wherein the cyclohexane polyalcohol compound is a scyllo-cyclohexanehexyl compound.

24. A dosage form according to any preceding claim wherein the cyclohexane polyalcohol compound is an epi-cyclohexanehexyl compound.

25. A method for treating Alzheimer's disease comprising administering a dosage form of any preceding claim to a patient in need thereof.

26. A method of preparing a stable dosage form of claim 1 comprising mixing an amount of a cyclohexane polyalcohol compound with a pharmaceutically acceptable carrier, excipient or diluent, the mixture being adapted to provide said mean plasma concentration profile.

27. Use of at least one cyclohexane polyalcohol compound for the preparation of a medicament to provide, when the medicament is administered in a dose of 500, 1000, 2000, 3500, 5000 or 7000 mg of said cyclohexane polyalcohol, a mean plasma concentration profile having a mean AUC_{0-INF} in μ·h/mL of, respectively, 43±20%, 130±20%, 215±20%, 467±20%, 507±20% or 885±20%, and having a mean Cₘₐₓ in μmL of, respectively, 5.8±20%, 17±20%, 33±20%, 75±20%, 110±20% or 155±20%, thereby preventing and/or treating a disorder in protein folding and/or aggregation, and/or amyloid formation, deposition, accumulation, or persistence.
